(19) United States
(12) Patent Application Publication
(10) Pub. No.: US 2023/0192876 A1
(43) Pub. Date: Jun. 22, 2023

(54) DOSING REGIMEN OF ANTI-CD27 ANTIBODIES FOR TREATMENT OF CANCER

(71) Applicant: Merck Sharp & Dohme Corp., Rahway, NJ (US)

(72) Inventors: Elliot K. Chartash, Basking Ridge, NJ (US); Amy M. Beebe, Half Moon Bay, CA (US); Jason K. Cheung, Westfield, NJ (US); Konstantin Dobrenkov, Rahway, NJ (US); Claire H. Li, Colmar, PA (US); Richard Wnek, Randolph, NJ (US)

(73) Assignee: Merck Sharp & Dohme Corp., Rahway, NJ (US)

(21) Appl. No.: 17/772,229

(22) PCT Filed: Oct. 29, 2020
(86) PCT No.: PCT/US2020/057817

§ 371 Date: Apr. 27, 2022

Related U.S. Application Data
(60) Provisional application No. 62/929,538, filed on Nov. 1, 2019.

(51) Int. Cl. C07K 16/28; A61P 35/00; A61K 39/395; A61K 31/282; A61K 31/519

(52) U.S. Cl. CPC C07K 16/2878; A61P 35/00; C07K 16/2818; C07K 16/2827; A61K 39/3955; A61K 31/282; A61K 31/519; A61K 2039/505

(57) ABSTRACT

The present invention relates to methods of treating cancer by administering an anti-CD27 antibody as a monotherapy or as a part of a combination thereof.

### CROSS-REFERENCE TO RELATED APPLICATIONS

This application claims priority from U.S. provisional application 62/929,538 filed Nov. 1, 2019, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Mar. 6, 2023, is named 25139-US-PCT_SL.txt and is 15,088 bytes in size.

### FIELD OF THE INVENTION

The present invention relates to dosing regimens of an anti-CD27 antibody or antigen binding fragment thereof useful for the treatment of cancer. In particular, the invention relates to the dosing regimen in a monotherapy comprising administering an anti-CD27 antibody or antigen binding fragment thereof and also the dosing regimen in a combination therapy which comprises administering an anti-CD27 antibody or antigen binding fragment thereof and at least one additional therapeutic agent, for example, an antibody directed to a Programmed Death 1 protein (PD-1) or Programmed Death Ligand 1 (PD-L1).

### BACKGROUND OF THE INVENTION

Cluster of differentiation (CD) 27, a tumor necrosis factor (TNF) receptor family super member was identified as a membrane molecule on human T cells (van Lier et al., 1987, J. Immunol. 139:1589-96). According to current evidence, CD27 has a single ligand, CD70, which is a TNF family member (Goodwin et al., 1993, Cell 73:447-56).

CD27 is exclusively expressed by hematopoietic cells, in particular those of the lymphocyte lineage, i.e., T lymphocyte (T cell), B-lymphocyte (B cell) and natural killer (NK) cells. CD27 was originally defined as a human T-cell co-stimulatory molecule that increments the proliferative response to T-Cell Receptor (TCR) stimulation (van Lier et al., 1987, J. Immunol. 139:1589-96). Presence of CD70, the ligand of CD27, dictates the timing and persistence of CD27-mediated co-stimulation.

Transgenic expression of CD70 in immature dendritic cells sufficed to convert immunological tolerance to virus or tumors into CD8+ T cell responsiveness. Likewise, agonistic soluble CD70 promoted the CD8+ T cell response upon such peptide immunization (Rowley et al., 2004, J Immunol 172:6039-6046) and in CD70 transgenic mice, CD4+ and CD8+ effector cell formation in response to TCR stimulation was greatly facilitated (Arens et al. 2001, Immunity 15:801-12; Tesselaar et al., 2003, Nat Immunol 4:49-54; Keller et al. 2008, Immunity 29: 334-346). In mouse lymphoma models, tumor rejection was improved upon CD70 transgenesis or injection of an anti-mouse CD27 antibody (Arens et al., 2003, J Exp Med 199:1595-1605; French et al., 2007, Blood 109: 4810-15; Sakanishi and Yagita, 2010, Biochem. Biophys. Res. Comm. 393: 829-835; and PCT publication numbers WO 2008/051424; WO 2012/004367).

In PCT publication number WO2012/004367 the first anti-human agonistic antibody (designated hCD27.15) was described that does not require crosslinking to activate CD27-mediated co-stimulation of the immune response. In addition, an anti-human CD27 antibody, designated 1F5 was disclosed that activates CD27 upon crosslinking (see PCT publication number WO2011/130434 and Vitale et al., Clin. Cancer Res, 2012, 18(14): 3812-3821). However, there is still a need in the art to develop anti-human CD27 antibodies having improved characteristics, including the ability to bind human CD27 having the A59T SNP and CD27 from cynomolgus monkeys.

Selecting a dosage regimen for an anti-CD27 antibody monotherapy or combination therapy with anti-PD-1 or anti-PD-L1 therapy depends on several factors, including the serum or tissue turnover rate of the entity, the level of symptoms, the immunogenicity of the entity, antidrug antibody endpoints and the accessibility of the target cells, tissue or organ in the individual being treated, as well as safety. Formation of antidrug antibodies can potentially confound drug exposures at therapeutic doses, and prime for subsequent infusion-related toxicities. In addition, anti-CD27 and/or anti-PD-1/anti-PD-L1 treatment can result in immune stimulation and the potential for cytokine release that affects safety.

### SUMMARY OF THE INVENTION

An aspect of the invention provides a method for treating cancer in a subject or a patient comprising administering to the subject or patient an anti-CD27 antibody or antigen binding fragment thereof. In various embodiments, the antibody is a human, humanized or chimeric antibody. In various embodiments, the antibody is an isolated antibody.

In various embodiments, the antibody or antigen binding fragment thereof comprises a heavy chain and a light chain, wherein the heavy chain comprises heavy chain complementarity determining regions (CDRs) of the amino acid sequence of SEQ ID NOs: 1, 2, and 3, and the light chain comprises light chain CDRs of the amino acid sequence of SEQ ID NOs: 4, 5, and 6. For example, the light chain comprises 3 CDRs and the heavy chain comprises three CDRs. In various embodiments of the method, heavy chain CDR1 comprises the amino acid sequence of SEQ ID NO.: 1, heavy chain CDR2 comprises the amino acid sequence of SEQ ID NO.:2, and heavy chain CDR3 comprises the amino acid sequence of SEQ ID NO.:3, and the light chain CDR1 comprises the amino acid sequence of SEQ ID NO.: 4, the light chain CDR2 comprises the amino acid sequence of SEQ ID NO.: 5, and the light chain CDR3 comprises the amino acid sequence of SEQ ID NO.: 6. For example, the heavy chain comprises heavy chain CDR1 consists of the amino acid sequence of SEQ ID NO.: 1, heavy chain CDR2 consists of the amino acid sequence of SEQ ID NO.: 2, and heavy chain CDR3 consists of the amino acid sequence of SEQ ID NO.: 3, and the light chain CDR1 consists of the amino acid sequence of SEQ ID NO.: 4, the light chain CDR2 consists of the amino acid sequence of SEQ ID NO.: 5, and the light chain CDR3 consists of the amino acid sequence of SEQ ID NO.: 6.

In various embodiments, the antibody or antigen binding fragment thereof comprises a heavy chain variable region and a light chain variable region. In various embodiments, the heavy chain variable region comprises three CDRs of the amino acid sequence of SEQ ID NOs: 1, 2, and 3, and the light chain comprises light chain three CDRs of the amino acid sequence of SEQ ID NOs: 4, 5, and 6.

In various embodiments of the method, the anti-CD27 antibody or antigen binding fragment thereof is administered via intravenous infusion. In various embodiments, the intravenous infusion comprises administering an analgesic and/or an antihistamine prior to the anti-CD27 antibody or antigen binding fragment thereof. In various embodiments, the analgesic and/or antihistamine is administered less than 3 hours, 2.5 hours, 2 hours, 1.5 hours, or 1 hour prior to administration of the anti-CD27 antibody or antigen binding fragment thereof. In various embodiments, the antihistamine is diphenhydramine. For example, diphenhydramine is administered at a dose of about 50 mg. For example, diphenhydramine is administered orally. In various embodiments, the analgesic is acetaminophen. For example, acetaminophen is administered at a dose of about 500 to about 1000 mg. In various embodiments, acetaminophen is administered orally.

In various embodiments of the method, the anti-CD27 antibody or antigen binding fragment thereof is administered once. In various embodiments of the method, the anti-CD27 antibody or antigen binding fragment thereof is administered at least once. In various embodiments of the method, the anti-CD27 antibody or antigen binding fragment thereof is administered multiple times, for example, 2, 3, 4, 5, or more times.

In various embodiments, the antibody or antigen binding fragment thereof is administered at one dose. For example, the subject or the patient is administered a dose of about 2 mg, about 7 mg, about 20 mg, about 30 mg, about 70 mg, about 200 mg, or about 700 mg. In various embodiments, the antibody or antigen binding fragment thereof is administered at multiple doses.

In various embodiments, the antibody or antigen binding fragment thereof is administered a dose of about 2 mg to about 700 mg. For example, the subject or patient is administered about 2 mg, about 7 mg, about 20 mg, about 30 mg, about 70 mg, about 200 mg, or about 700 mg. In various embodiments of the method, the subject or the patient is administered 2 mg of the anti-CD27 antibody or antigen binding fragment thereof. In various embodiments of the method, the subject or the patient is administered 7 mg of the anti-CD27 antibody or antigen binding fragment thereof. In various embodiments of the method, the subject or the patient is administered 20 mg of the anti-CD27 antibody or antigen binding fragment thereof. In various embodiments of the method, the subject or the patient is administered 30 mg of the anti-CD27 antibody or antigen binding fragment thereof. In various embodiments of the method, the subject or the patient is administered 70 mg of the anti-CD27 antibody or antigen binding fragment thereof. In various embodiments of the method, the subject or the patient is administered 200 mg of the anti-CD27 antibody or antigen binding fragment thereof. In various embodiments of the method, the subject or the patient is administered 700 mg of the anti-CD27 antibody or antigen binding fragment thereof. In various embodiments of the method, the subject or the patient is administered a dose of about 200 mg to about 700 mg of the anti-CD27 antibody or antigen binding fragment thereof. In various embodiments of the method, the subject or the patient is administered a dose of about 30 mg to about 700 mg of the anti-CD27 antibody or antigen binding fragment thereof. In various embodiments of the method, the subject or the patient is administered a dose of about 30 mg, about 200 mg, or about 200 mg to about 700 mg of the anti-CD27 antibody or antigen binding fragment. In various embodiments of the method, the subject or the patient is administered a dose of about 30 mg to about 200 mg of the anti-CD27 antibody or antigen binding fragment thereof. In various embodiments of the method, the subject or the patient is administered a dose of about 70 mg to about 200 mg of the anti-CD27 antibody or antigen binding fragment thereof.

In various embodiments of the method, the subject or the patient is administered the anti-CD27 antibody or antigen binding fragment thereof on Day 1 and then once every three weeks thereafter. In various embodiments, the subject or the patient is administered the anti-CD27 antibody or antigen binding fragment thereof as a monotherapy. In various embodiments, the subject or the patient is administered the anti-CD27 antibody or antigen binding fragment thereof as a combination therapy.

In various embodiments of the method, the anti-CD27 antibody or antigen binding fragment thereof comprises a heavy chain and a light chain, and wherein the heavy chain comprises a heavy chain variable region comprising the amino acid sequence of SEQ ID NO.: 7 and the light chain comprises a light chain variable region comprising the amino acid sequence of SEQ ID NO.: 9.

In various embodiments of the method, the anti-CD27 antibody or antigen binding fragment thereof comprises a heavy chain and a light chain, and wherein the heavy chain comprises the amino acid sequence of SEQ ID NO.: 8 and the light chain comprises the amino acid sequence of SEQ ID NO.: 10.

In various embodiments of the method, the anti-CD27 antibody is administered to the subject or patient. In various embodiments, the subject or the patient is administered the anti-CD27 antibody as a monotherapy. In various embodiments, the subject or the patient is administered the anti-CD27 antibody as a combination therapy. For example, the anti-CD27 antibody or antigen binding fragment thereof is co-administered with an anti-PD-1 antibody or anti-PD-L1 antibody or antigen binding fragment thereof. In various embodiments of the method, the anti-CD27 antibody is co-formulated with an anti-PD-1 antibody or anti-PD-L1 antibody or antigen binding fragment thereof.

In various embodiments of the method, the anti-PD-1 antibody, or antigen binding fragment thereof, specifically binds to human PD-1 and blocks the binding of human PD-L1 to human PD-1. In various embodiments of the method, the anti-PD-1 antibody, or antigen binding fragment thereof, also blocks binding of human PD-L2 to human PD-1. In various embodiments of the method, the anti-PD-1 antibody, or antigen binding fragment thereof comprises: (a) heavy chain CDRs comprising the amino acid sequences of SEQ ID NOs: 16, 17 and 18 and (b) light chain CDRs comprising the amino acid sequences of SEQ ID NOs: 11, 12 and 13.

In various embodiments of the method, the anti-PD-1 antibody or antigen binding fragment thereof comprises a heavy chain and a light chain, and wherein the heavy chain comprises a heavy chain variable region comprising the amino acid sequence of SEQ ID NO.: 19 and the light chain comprises a light chain variable region comprising the amino acid sequence of SEQ ID NO.: 14. In various embodiments of the method, the anti-PD-1 antibody comprises a heavy chain and a light chain, and wherein the heavy chain comprises the amino acid sequence of SEQ ID NO.: 20 and the light chain comprises the amino acid sequence of SEQ ID NO.: 15.

In various embodiments of the method, the anti-PD-1 antibody is pembrolizumab. In various embodiments of the method, the anti-PD-1 antibody is a pembrolizumab variant.

In various embodiments of the method, the anti-PD-1 antibody is nivolumab. In various embodiments of the method, the anti-PD-L1 antibody is atezolizumab, durvalumab, or avelumab.

In various embodiments of the method, the anti-PD-1 antibody is administered at 200 mg via intravenous infusion on Day 1 and then once every three weeks thereafter. In various embodiments of the method, the anti-PD-1 antibody is administered at 400 mg via intravenous infusion on Day 1 and then once every six weeks thereafter.

In various embodiments of the method, the anti-PD-1 antibody is a humanized anti-PD-1 antibody that comprises a heavy chain and a light chain, and wherein the light chain comprises a light chain variable region comprising light chain CDRs comprising the amino acid sequences of SEQ ID NOs: 11, 12 and 13, and the heavy chain comprises a heavy chain variable region comprising heavy chain CDRs comprising the amino acid sequences of SEQ ID NOs:16, 17 and 18 and the anti-CD27 antibody is a humanized anti-CD27 antibody which comprises a heavy chain and a light chain, and wherein the heavy chain comprises a heavy chain variable region comprising heavy chain CDRs comprising the amino acid sequences of SEQ ID NOs: 1, 2 and 3, and the light chain comprises a light chain variable region comprising light chain CDRs comprising the amino acid sequences of SEQ ID NOs: 4, 5 and 6.

In various embodiments of the method, the anti-PD-1 antibody comprises a heavy chain and a light chain, and wherein the heavy chain comprises a heavy chain variable region comprising the amino acid sequence of SEQ ID NO.:19 and the light chain comprises a light chain variable region comprising the amino acid sequence of SEQ ID NO.: 14; and the anti-CD27 antibody comprises a heavy chain and a light chain, and wherein the heavy chain comprises a heavy chain variable region comprising the amino acid sequence of SEQ ID NO.: 7 and the light chain comprises a light chain variable region comprising the amino acid sequence of SEQ ID NO.: 9.

In various embodiments of the method, the anti-PD-1 antibody comprises a heavy chain and a light chain, and wherein the heavy chain comprises the amino acid sequence of SEQ ID NO.: 20 and the light chain comprises the amino acid sequence of SEQ ID NO.: 15; and the anti-CD27 antibody comprises a heavy chain and a light chain, and wherein the heavy chain comprises the amino acid sequence of SEQ ID NO.: 8 and the light chain comprises a light chain variable region comprising the amino acid sequence of SEQ ID NO.: 10.

In various embodiments of the method, the anti-PD-1 antibody is administered at 200 mg via intravenous infusion on Day 1 and then once every three weeks thereafter, and the anti-CD27 antibody is administered at 200 mg via intravenous infusion on Day 1 and then once every three weeks thereafter.

In various embodiments of the method, the subject or the patient is administered the anti-CD27 antibody or antigen binding fragment thereof on Day 1 and then at least once a few or several weeks afterwards. For example, the subject or the patient is administered the anti-CD27 antibody or antigen binding fragment thereof on Day 1 and about 3 weeks to about 6 weeks thereafter. In various embodiments of the method, the subject or the patient is administered the anti-CD27 antibody or antigen binding fragment thereof on Day 1 and then every 3 weeks. In various embodiments of the method, the subject or the patient is administered the anti-CD27 antibody or antigen binding fragment thereof on Day 1 and then every 6 weeks.

In various embodiments of the method, the anti-CD27 antibody or antigen binding fragment thereof comprises a heavy chain and a light chain, and wherein the heavy chain comprises a heavy chain variable region comprising the amino acid sequence of SEQ ID NO.:

7 and the light chain comprises a light chain variable region comprising the amino acid sequence of SEQ ID NO.: 9.

In various embodiments of the method, the anti-CD27 antibody or antigen binding fragment thereof comprises a heavy chain and a light chain, and wherein the heavy chain comprises the amino acid sequence of SEQ ID NO.: 8 and the light chain comprises the amino acid sequence of SEQ ID NO.: 10.

In various embodiments of the method, the anti-CD27 antibody or antigen binding fragment thereof is co-administered with an anti-PD-1 antibody or anti-PD-L1 antibody or antigen binding fragment thereof.

In various embodiments of the method, the anti-CD27 antibody or antigen binding fragment thereof is co-formulated with an anti-PD-1 antibody or anti-PD-L1 antibody or antigen binding fragment thereof. In various embodiments of the method, the anti-PD-1 antibody or antigen binding fragment thereof, specifically binds to human PD-1 and blocks the binding of human PD-L1 to human PD-1.

In various embodiments of the method, the anti-PD-1 antibody or antigen binding fragment thereof, also blocks binding of human PD-L2 to human PD-1. In various embodiments of the method, the anti-PD-1 antibody, or antigen binding fragment thereof comprises: (a) light chain CDRs comprising the amino acid sequences of SEQ ID NOs: 11, 12 and 13 and (b) heavy chain CDRs comprising the amino acid sequences of SEQ ID NOs: 16, 17 and 18.

In various embodiments of the method, the anti-PD-1 antibody comprises a heavy chain and a light chain, and wherein the heavy chain comprises a heavy chain variable region comprising the amino acid sequence of SEQ ID NO.:19 and the light chain comprises a light chain variable region comprising the amino acid sequence of SEQ ID NO.: 14.

In various embodiments of the method, the anti-PD-1 antibody comprises a heavy chain and a light chain, and wherein the heavy chain comprises the amino acid sequence of SEQ ID NO.: 20 and the light chain comprises the amino acid sequence of SEQ ID NO.: 15.

In various embodiments of the method, the anti-PD-1 antibody is pembrolizumab. In various embodiments of the method, the anti-PD-1 antibody is a pembrolizumab variant.

In various embodiments of the method, the anti-PD-1 antibody is nivolumab.

In various embodiments of the method, the anti-PD-1 antibody and the anti-CD27 antibody or antigen binding fragment thereof are co-administered. In various embodiments of the method, the anti-PD-1 antibody and the anti-CD27 antibody or antigen binding fragment thereof are co-formulated.

In various embodiments of the method, the anti-PD-L1 antibody is atezolizumab, durvalumab, or avelumab.

In various embodiments of the method, the anti-CD27 antibody or antigen binding fragment thereof is administered every 3 weeks (Q3W).

In various embodiments of the method, the anti-CD27 antibody or antigen binding fragment thereof is administered every 6 weeks (Q6W).

In various embodiments, the anti-CD27 antibody or antigen binding fragment thereof is administered as an infusion. In various embodiments, the anti-CD27 antibody or antigen binding fragment thereof is administered as an infusion over a period of time. For example, the period of time is at least about 30 minutes, about 45 minutes, about 60 minutes, or about 90 minutes. In various embodiments, the period of time is 90 minutes.

In various embodiments, the anti-PD-1 antibody or antigen binding fragment thereof is administered as an infusion. In various embodiments, the anti-CD27 antibody or antigen binding fragment thereof is administered as an infusion over a period of time. For example, the period of time is at least about 30 minutes. In various embodiments, the period of time is 30 minutes.

In various embodiments, the anti-CD27 antibody or antigen binding fragment thereof is administered from a vial. For example, the volume of the vial is about 1 milliliter (mL or ml).

In various embodiments, the concentration of the anti-CD27 antibody or antigen binding fragment thereof in the vial is about 50 mg/ml.

In various embodiments of the method, the anti-PD-1 antibody is administered at 200 mg via intravenous infusion on Day 1 and then once every three weeks thereafter. In various embodiments of the method, the anti-PD-1 antibody is administered at 400 mg via intravenous infusion on Day 1 and then once every six weeks thereafter.

In various embodiments of the method, the anti-CD27 antibody is administered to the subject or patient. In various embodiments of the method, the anti-CD27 antigen binding fragment thereof is administered to the subject or patient.

In various embodiments of the method, the anti-PD-1 antibody is a humanized anti-PD-1 antibody that comprises a heavy chain and a light chain, and wherein the heavy chain comprises a heavy chain variable region comprising heavy chain CDRs comprising the amino acid sequences of SEQ ID NOs: 16, 17 and 18, and the light chain comprises a light chain variable region comprising light chain CDRs comprising the amino acid sequences of SEQ ID NOs: 11, 12 and 13; and the anti-CD27 antibody is a humanized anti-CD27 antibody which comprises a heavy chain and a light chain, and wherein the heavy chain comprises a heavy chain variable region comprising heavy chain CDRs comprising the amino acid sequences of SEQ ID NOs: 1, 2 and 3, and the light chain comprises a light chain variable region comprising light chain CDRs comprising the amino acid sequences of SEQ ID NOs: 4, 5 and 6.

In various embodiments of the method, the anti-PD-1 antibody comprises a heavy chain and a light chain, and wherein the heavy chain comprises a heavy chain variable region comprising the amino acid sequences of SEQ ID NO.: 19 and the light chain comprises a light chain variable region comprising the amino acid sequences of SEQ ID NO.: 14; and the anti-CD27 antibody comprises a heavy chain and a light chain, and wherein the heavy chain comprises a heavy chain variable region comprising the amino acid sequence of SEQ ID NO.: 7 and the light chain comprises a light chain variable region comprising the amino acid sequence of SEQ ID NO.: 9.

In various embodiments of the method, the anti-PD-1 antibody comprises a heavy chain and a light chain, and wherein the heavy chain comprises the amino acid sequence of SEQ ID NO.: 20 and the light chain comprises the amino acid sequence of SEQ ID NO.: 15; and the anti-CD27 antibody comprises a heavy chain and a light chain, and wherein the heavy chain comprises the amino acid sequence of SEQ ID NO.: 8 and the light chain comprises a light chain variable region comprising the amino acid sequence of SEQ ID NO.: 10.

In various embodiments of the method, the anti-PD-1 antibody is administered at 200 mg via intravenous infusion on Day 1 and then once every three weeks thereafter, and the anti-CD27 antibody is administered at 30 mg via intravenous infusion on Day 1 and then once every three weeks thereafter.

In various embodiments of the method, the anti-PD-1 antibody is administered at 400 mg via intravenous infusion on Day 1 and then once every six weeks thereafter, and the anti-CD27 antibody is administered at 30 mg via intravenous infusion on Day 1 once every six weeks.

In various embodiments of the method, the anti-PD-1 antibody is co-formulated with the anti-CD27 antibody.

In various embodiments of the method, the cancer is a solid tumor cancer and/or is characterized by presence of at least one solid tumor or a plurality of solid tumors. In various embodiments of the method, the cancer is characterized by the presence of at least one advanced solid tumor. In various embodiments of the method, the cancer is selected from the group consisting of: triple-negative breast cancer (TNBC), non-squamous non-small cell lung cancer (NSCLC), and endometrial cancer.

In various embodiments, the method further comprises administering carboplatin and/or pemetrexed. In various embodiments, the method further comprises administering a combination of carboplatin and pemetrexed. For example, the carboplatin and pemetrexed is administered after the anti-CD27 antibody or antigen binding fragment thereof (along or in combination with the PD-1 antibody or antigen binding fragment).

In various embodiments of the method, the subject or the patient has not been previously treated with anti-PD-1 or anti-PD-L1 therapy or is confirmed progressive while receiving prior anti-PD-1 or anti-PD-L1 therapy.

In various embodiments of the method, the patient is premedicated prior to administering the antibody or antigen binding fragment thereof. In various embodiments, the premedication comprises administering an analgesic and/or an antihistamine prior to the anti-CD27 antibody or antigen binding fragment thereof. In various embodiments, the analgesic and/or antihistamine is administered less than 3 hours, 2.5 hours, 2 hours, 1.5 hours, or 1 hour prior to administration of the anti-CD27 antibody or antigen binding fragment thereof. In various embodiments, the antihistamine is diphenhydramine. For example, the premedicated comprises administering diphenhydramine at a dose of about 50 mg. For example, administering is an oral administration. In various embodiments, the analgesic is acetaminophen. For example, the premedication comprises administering acetaminophen at a dose of about 500 to about 1000 mg. In various embodiments, the administering is an oral administration.

An aspect of the invention provides an anti-CD27 antibody or antigen binding fragment thereof for use in treating cancer in a subject or a patient. In various embodiments, the anti-CD27 antibody or antigen binding fragment thereof comprises a heavy chain and a light chain, wherein the heavy chain comprises heavy chain CDRs comprising the amino acid sequences of SEQ ID NOs: 1, 2 and 3, and the light chain comprises light chain CDRs comprising the amino acid sequence of SEQ ID NOs: 4, 5 and 6. For example, the light chain comprises 3 CDRs and the heavy chain comprises three CDRs. In various embodiments of the use, the heavy chain CDR1 comprises the amino acid sequence of SEQ ID NO.: 1, the heavy chain CDR2 comprises the amino acid sequence of SEQ ID NO.: 2, and the heavy chain CDR3 comprises the amino acid sequence of SEQ ID NO.: 3, and the light chain CDR1 comprises the amino acid sequence of SEQ ID NO.: 4, the light chain CDR2 comprises the amino acid sequence of SEQ ID NO.: 5, and the light chain CDR3 comprises the amino acid sequence of SEQ ID NO.: 6. For example, the heavy chain CDR1 consists of the amino acid sequence of SEQ ID NO.: 1, the heavy chain CDR2 consists of the amino acid sequence of SEQ ID NO.: 2, and the heavy chain CDR3 consists of the amino acid sequence of SEQ ID NO.: 3, and the light chain CDR1 consists of the amino acid sequence of SEQ ID NO.: 4, the light chain CDR2 consists of the amino acid sequence of SEQ ID NO.: 5, and the light chain CDR3 consists of the amino acid sequence of SEQ ID NO.: 6.

In various embodiments, the anti-CD27 antibody or antigen binding fragment thereof comprises a heavy chain variable region and a light chain variable region, wherein the heavy chain variable region comprises heavy chain CDRs comprising the amino acid sequences of SEQ ID NOs: 1, 2 and 3, and the light chain variable region comprises light chain CDRs comprising the amino acid sequence of SEQ ID NOs: 4, 5 and 6. For example, the light chain variable region comprises 3 CDRs and the heavy chain variable region comprises three CDRs. In various embodiments of the use, the heavy chain variable region CDR1 comprises the amino acid sequence of SEQ ID NO.: 1, the heavy chain variable region CDR2 comprises the amino acid sequence of SEQ ID NO.: 2, and the heavy chain variable region CDR3 comprises the amino acid sequence of SEQ ID NO.: 3, and the light chain variable region CDR1 comprises the amino acid sequence of SEQ ID NO.: 4, the light chain variable region CDR2 comprises the amino acid sequence of SEQ ID NO.: 5, and the light chain variable region CDR3 comprises the amino acid sequence of SEQ ID NO.: 6. For example, the heavy chain variable region CDR1 consists of the amino acid sequence of SEQ ID NO.: 1, the heavy chain variable region CDR2 consists of the amino acid sequence of SEQ ID NO.: 2, and the heavy chain variable region CDR3 consists of the amino acid sequence of SEQ ID NO.: 3, and the light chain variable region CDR1 consists of the amino acid sequence of SEQ ID NO.: 4, the light chain variable region CDR2 consists of the amino acid sequence of SEQ ID NO.: 5, and the light chain variable region CDR3 consists of the amino acid sequence of SEQ ID NO.: 6.

In various embodiments, the anti-CD27 antibody or antigen binding fragment thereof is administered via intravenous infusion. In various embodiments, the anti-CD27 antibody or antigen binding fragment thereof is formulated for intravenous infusion. In various embodiments, the anti-CD27 antibody or antigen binding fragment thereof is administered once.

In various embodiments, the anti-CD27 antibody or antigen binding fragment thereof is administered multiple times. In various embodiments, the anti-CD27 antibody or antigen binding fragment thereof is administered Q3W. In various embodiments, the anti-CD27 antibody or antigen binding fragment thereof is administered Q6W.

In various embodiments, the intravenous infusion comprises administering an analgesic and/or an antihistamine prior to the anti-CD27 antibody or antigen binding fragment thereof. In various embodiments, the antihistamine is diphenhydramine. In various embodiments, the analgesic and/or antihistamine is administered less than 3 hours, 2.5 hours, 2 hours, 1.5 hours, or 1 hour prior to administration of the anti-CD27 antibody or antigen binding fragment thereof. For example, diphenhydramine is administered at a dose of about 50 mg. For example, diphenhydramine is administered orally. In various embodiments, the analgesic is acetaminophen. For example, acetaminophen is administered at a dose of about 500 to about 1000 mg. In various embodiments, acetaminophen is administered orally.

In various embodiments, the antibody or antigen binding fragment thereof is administered at one dose. For example, the subject or the patient is administered a dose of about 2 mg, about 7 mg, about 20 mg, about 30 mg, about 70 mg, about 200 mg, or about 700 mg. In various embodiments, the antibody or antigen binding fragment thereof is administered at multiple doses. For example, the subject or the patient is administered for such use multiple doses that total about 2 mg, about 7 mg, about 20 mg, about 30 mg, about 70 mg, about 200 mg, or about 700 mg.

In various embodiments, the antibody or antigen binding fragment thereof is administered a dose of about 2 mg to about 700 mg. For example, the subject or the patient is administered for such use about 2 mg, about 7 mg, about 20 mg, about 30 mg, about 70 mg, about 200 mg, or about 700 mg of the antibody or antigen binding fragment thereof. In various embodiments, the subject or the patient is administered 2 mg of the anti-CD27 antibody or antigen binding fragment thereof. In various embodiments, the subject or the patient is administered 7 mg of the anti-CD27 antibody or antigen binding fragment thereof. In various embodiments, the subject or the patient is administered 20 mg of the anti-CD27 antibody or antigen binding fragment thereof. In various embodiments, the subject or the patient is administered 30 mg of the anti-CD27 antibody or antigen binding fragment thereof. In various embodiments, the subject or the patient is administered 70 mg of the anti-CD27 antibody or antigen binding fragment thereof. In various embodiments, the subject or the patient is administered 200 mg of the anti-CD27 antibody or antigen binding fragment thereof. In various embodiments of the use, the subject or the patient is administered 700 mg of the anti-CD27 antibody or antigen binding fragment thereof. In various embodiments, the subject or the patient is administered a dose of about 200 mg to about 700 mg of the anti-CD27 antibody or antigen binding fragment thereof. In various embodiments, the subject or the patient is administered a dose of about 30 mg to about 700 mg of the anti-CD27 antibody or antigen binding fragment thereof. In various embodiments, wherein the subject or the patient is administered a dose of about 30 mg, about 200 mg, or about 200 mg to about 700 mg of the anti-CD27 antibody or antigen binding fragment thereof. In various embodiments, the subject or the patient is administered a dose of about 30 mg to about 200 mg of the anti-CD27 antibody or antigen binding fragment thereof.

In various embodiments of the use, the subject or the patient is administered the anti-CD27 antibody or antigen binding fragment thereof for such use on Day 1 and then once every three weeks thereafter. In various embodiments, the subject or the patient is administered the anti-CD27 antibody or antigen binding fragment thereof as a monotherapy. In various embodiments, the subject or the patient is administered the anti-CD27 antibody or antigen binding fragment thereof as a combination therapy.

In various embodiments, the anti-CD27 antibody or antigen binding fragment thereof for such use comprises a heavy chain and a light chain, and wherein the heavy chain comprises a heavy chain variable region comprising the amino acid sequence of SEQ ID NO.: 7 and the light chain comprises a light chain variable region comprising the amino acid sequence of SEQ ID NO.: 9.

In various embodiments, the anti-CD27 antibody or antigen binding fragment thereof comprises a heavy chain and a light chain, and wherein the heavy chain comprises the amino acid sequence of SEQ ID NO.: 8 and the light chain comprises the amino acid sequence of SEQ ID NO.:10.

In various embodiments, the anti-CD27 antibody or antigen binding fragment for such use is administered to the subject or the patient. In various embodiments, the subject or the patient is administered the anti-CD27 antibody or the antigen binding fragment as a monotherapy. In various embodiments, the subject or the patient is administered the anti-CD27 antibody or antigen binding fragment as a combination therapy. For example, the anti-CD27 antibody or antigen binding fragment thereof is co-administered with an anti-PD-1 antibody or anti-PD-L1 antibody or antigen binding fragment thereof. In various embodiments of the use, the anti-CD27 antibody or antigen binding fragment thereof is co-formulated with an anti-PD-1 antibody or anti-PD-L1 antibody or antigen binding fragment thereof.

In various embodiments, the anti-PD-1 antibody or antigen binding fragment thereof, for such use specifically binds to human PD-1 and blocks the binding of human PD-L1 to human PD-1. In various embodiments, the anti-PD-1 antibody, or antigen binding fragment thereof, also blocks binding of human PD-L2 to human PD-1. In various embodiments, the anti-PD-1 antibody or antigen binding fragment thereof comprises: (a) heavy chain CDRs comprising the amino acid sequences of SEQ ID NOs: 16, 17 and 18 and (b) light chain CDRs comprising the amino acid sequences of SEQ ID NOs: 11, 12 and 13.

In various embodiments, the anti-PD-1 antibody comprises a heavy chain and a light chain, and wherein the heavy chain comprises a heavy chain variable region comprising the amino acid sequence of SEQ ID NO.: 19 and the light chain comprises a light chain variable region comprising the amino acid sequence of SEQ ID NO.: 14. In various embodiments, the anti-PD-1 antibody comprises a heavy chain and a light chain, and wherein the heavy chain comprises the amino acid sequence of SEQ ID NO.: 20 and the light chain comprises the amino acid sequence of SEQ ID NO.: 15.

In various embodiments, the anti-PD-1 antibody for such use is pembrolizumab. In various embodiments, the anti-PD-1 antibody for such use is a pembrolizumab variant.

In various embodiments, the anti-PD-1 antibody or antigen binding fragment thereof and the anti-CD27 antibody or antigen binding fragment thereof for such use are co-administered. In various embodiments, the anti-PD-1 antibody or antigen binding fragment thereof and the anti-CD27 antibody or antigen binding fragment thereof for such use are co-formulated. In various embodiments of the use, the anti-PD-1 antibody for such use is nivolumab. In various embodiments, the anti-PD-L1 antibody for such use is atezolizumab, durvalumab, or avelumab.

In various embodiments, the anti-PD-1 antibody or antigen binding fragment thereof is administered at 200 mg via intravenous infusion on Day 1 and then once every three weeks thereafter. In various embodiments, the anti-PD-1 antibody or antigen binding fragment thereof is administered at 400 mg via intravenous infusion on Day 1 and then once every six weeks thereafter.

In various embodiments, the anti-PD-1 antibody for such use is a humanized anti-PD-1 antibody that comprises a heavy chain and a light chain, and wherein the light chain comprises a light chain variable region comprising heavy chain CDRs comprising the amino acid sequences of SEQ ID NOs: 11, 12 and 13. and the heavy chain comprises a heavy chain variable region comprising light chain CDRs comprising the amino acid sequences of SEQ ID NOs: 16, 17 and 18 and the anti-CD27 antibody is a humanized anti-CD27 antibody which comprises a heavy chain and a light chain, and wherein the heavy chain comprises a heavy chain variable region comprising heavy chain CDRs comprising the amino acid sequences of SEQ ID NOs: 1, 2 and 3. and the light chain comprises a light chain variable region comprising light chain CDRs comprising the amino acid sequences of SEQ ID NOs: 4, 5 and 6.

In various embodiments, the anti-PD-1 antibody comprises a heavy chain and a light chain, and wherein the heavy chain comprises a heavy chain variable region comprising the amino acid sequence of SEQ ID NO.:19 and the light chain comprises a light chain variable region comprising the amino acid sequence of SEQ ID NO.: 14; and the anti-CD27 antibody comprises a heavy chain and a light chain, and wherein the heavy chain comprises a heavy chain variable region comprising the amino acid sequence of SEQ ID NO.: 7 and the light chain comprises a light chain variable region comprising the amino acid sequence of SEQ ID NO.: 9.

In various embodiments, the anti-PD-1 antibody for such use comprises a heavy chain and a light chain, and wherein the heavy chain comprises the amino acid sequence of SEQ ID NO.: 15 and the light chain comprises the amino acid sequence of SEQ ID NO.: 20; and the anti-CD27 antibody comprises a heavy chain and a light chain, and wherein the heavy chain comprises the amino acid sequence of SEQ ID NO.: 8 and the light chain comprises a light chain variable region comprising the amino acid sequence of SEQ ID NO.: 10.

In various embodiments, the anti-PD-1 antibody or antigen binding fragment thereof for such use is administered at 200 mg via intravenous infusion on Day 1 and then once every three weeks thereafter, and the anti-CD27 antibody or antigen binding fragment thereof is administered at 200 mg via intravenous infusion on Day 1 and then once every three weeks thereafter.

In various embodiments, the subject or the patient is administered the anti-CD27 antibody or antigen binding fragment thereof on Day 1 and then at least once a week or a number of weeks afterwards. For example, the subject or the patient is administered the anti-CD27 antibody or antigen binding fragment thereof on Day 1 and about 3 weeks to about 6 weeks thereafter.

In various embodiments, the anti-CD27 antibody or antigen binding fragment thereof comprises a heavy chain and a light chain, and wherein the heavy chain comprises a heavy chain variable region comprising the amino acid sequence of SEQ ID NO.: 7 and the light chain comprises a light chain variable region comprising the amino acid sequence of SEQ ID NO.: 9.

In various embodiments, the anti-CD27 antibody or antigen binding fragment thereof comprises a heavy chain and a light chain, and wherein the heavy chain comprises the amino acid sequence of SEQ ID NO.: 8 and the light chain comprises the amino acid sequence of SEQ ID NO.: 10.

In various embodiments, the anti-CD27 antibody or antigen binding fragment thereof is co-administered with an anti-PD-1 antibody or antigen binding fragment thereof or anti-PD-L1 antibody or antigen binding fragment thereof.

In various embodiments, the anti-CD27 antibody or antigen binding fragment thereof is administered with an anti-PD-1 antibody or antigen binding fragment thereof or anti-PD-L1 antibody or antigen binding fragment thereof.

In various embodiments, the anti-CD27 antibody or antigen binding fragment thereof is co-formulated with an anti-PD-1 antibody or antigen binding fragment thereof or anti-PD-L1 antibody or antigen binding fragment thereof.

In various embodiments, the anti-PD-1 antibody or antigen binding fragment thereof for such use specifically binds to human PD-1 and blocks the binding of human PD-L1 to human PD-1.

In various embodiments, the anti-PD-1 antibody or antigen binding fragment thereof, for such use also blocks binding of human PD-L2 to human PD-1.

In various embodiments, the anti-PD-1 antibody or antigen binding fragment thereof comprises: (a) light chain CDRs comprising the amino acid sequences of SEQ ID NOs: 11, 12 and 13, and (b) heavy chain CDRs of comprising the amino acid sequences SEQ ID NOs: 16, 17 and 18.

In various embodiments, the anti-PD-1 antibody or antigen binding fragment thereof comprises a heavy chain and a light chain, and wherein the heavy chain comprises a heavy chain variable region comprising the amino acid sequence of SEQ ID NO.:19 and the light chain comprises a light chain variable region comprising SEQ ID NO.: 14.

In various embodiments, the anti-PD-1 antibody or antigen binding fragment thereof for such use comprises a heavy chain and a light chain, and wherein the heavy chain comprises the amino acid sequence of SEQ ID NO.: 20 and the light chain comprises the amino acid sequence of SEQ ID NO.: 15.

In various embodiments, the anti-PD-1 antibody for such use is pembrolizumab. In various embodiments, the anti-PD-1 antibody for such use is a pembrolizumab variant.

In various embodiments, the anti-PD-1 antibody for such use is nivolumab.

In various embodiments, the anti-PD-L1 antibody for such use is atezolizumab, durvalumab, or avelumab.

In various embodiments, the anti-CD27 antibody or antigen binding fragment thereof for such use is administered Q3W.

In various embodiments, the anti-CD27 antibody or antigen binding fragment thereof for such use is administered Q6W.

In various embodiments, the anti-PD-1 antibody or antigen binding fragment thereof for such use is administered at 200 mg via intravenous infusion on Day 1 and then once every three weeks thereafter. In various embodiments, the anti-PD-1 antibody or antigen binding fragment thereof for such use is administered at 400 mg via intravenous infusion on Day 1 and then once every six weeks thereafter.

In various embodiments, the anti-CD27 antibody or antigen binding fragment thereof for such use is administered to the subject or the patient. In various embodiments, the anti-CD27 antigen binding fragment thereof for such use is administered to the subject or the patient.

In various embodiments, the anti-PD-1 antibody for such use is a humanized anti-PD-1 antibody that comprises a heavy chain and a light chain, and wherein the heavy chain comprises a heavy chain variable region comprising heavy chain CDRs comprising the amino acid sequences of SEQ ID NOs: 16, 17 and 18, and the light chain comprises a light chain variable region comprising light chain CDRs comprising the amino acid sequences of SEQ ID NOs: 11, 12 and 13; and the anti-CD27 antibody is a humanized anti-CD27 antibody which comprises a heavy chain and a light chain, and wherein the heavy chain comprises a heavy chain variable region comprising heavy chain CDRs comprising the amino acid sequences of SEQ ID NOs: 1, 2 and 3, and the light chain comprises a light chain variable region comprising light chain CDRs comprising the amino acid sequences of SEQ ID NOs: 4, 5 and 6.

In various embodiments, the anti-PD-1 antibody or antigen binding fragment thereof for such use comprises a heavy chain and a light chain, and wherein the heavy chain comprises a heavy chain variable region comprising the amino acid sequence of SEQ ID NO.: 19 and the light chain comprises a light chain variable region comprising the amino acid sequence of SEQ ID NO.: 14; and the anti-CD27 antibody or antigen binding fragment thereof comprises a heavy chain and a light chain, and wherein the heavy chain comprises a heavy chain variable region comprising the amino acid sequence of SEQ ID NO.: 7 and the light chain comprises a light chain variable region comprising the amino acid sequence of SEQ ID NO.: 9.

In various embodiments, the anti-PD-1 antibody or antigen binding fragment thereof for such use comprises a heavy chain and a light chain, and wherein the heavy chain comprises the amino acid sequence of SEQ ID NO.: 20 and the light chain comprises the amino acid sequence of SEQ ID NO.: 15; and the anti-CD27 antibody or antigen binding fragment thereof comprises a heavy chain and a light chain, and wherein the heavy chain comprises the amino acid sequence of SEQ ID NO.: 8 and the light chain comprises a light chain variable region comprising the amino acid sequence of SEQ ID NO.: 10.

In various embodiments, the anti-PD-1 antibody or antigen binding fragment thereof for such use is administered at 200 mg via intravenous infusion on Day 1 and then once every three weeks thereafter, and the anti-CD27 antibody or antigen binding fragment thereof is administered at 30 mg via intravenous infusion on Day 1 and then once every three weeks thereafter.

In various embodiments, the anti-PD-1 antibody or antigen binding fragment thereof for such use is administered at 400 mg via intravenous infusion on Day 1 and then once every six weeks thereafter, and the anti-CD27 antibody or antigen binding fragment thereof is administered at 30 mg via intravenous infusion on Day 1 once every six weeks.

In various embodiments, the anti-PD-1 antibody or antigen binding fragment thereof for such use is co-formulated with the anti-CD27 antibody or antigen binding fragment thereof. In various embodiments, the anti-PD-1 antibody or antigen binding fragment thereof for such use is co-administered with the anti-CD27 antibody or antigen binding fragment thereof.

In various embodiments, the cancer for such use is characterized by presence of at least one solid tumor. For example, the at least one solid tumor is an advanced solid tumor. In various embodiments, the cancer is selected from the group consisting of: TNBC, NSCLC, and endometrial cancer.

In various embodiments, the use further comprises carboplatin and/or pemetrexed for such use. In various embodiments, the use further comprises a combination of carboplatin and pemetrexed for such use.

In various embodiments, the subject or the patient for such use has not been previously treated with anti-PD-1 or anti-PD-L1 therapy or is confirmed progressive while receiving prior anti-PD-1 or anti-PD-L1 therapy.

An aspect of the invention provides a pharmaceutical composition comprising about 2 mg to about 700 mg of the anti-CD27 antibody or antigen binding fragment thereof described herein (e.g., the anti-CD27 antibody or antigen binding fragment thereof comprises at least one sequence described in Table 1), and a pharmaceutically acceptable excipient. For example, the subject or the patient is administered about 2 mg, about 7 mg, about 20 mg, about 30 mg, about 70 mg, about 200 mg, or about 700 mg of the antibody or antigen binding fragment thereof. In various embodiments, the pharmaceutical composition further comprises anti-PD-1 antibody or anti-PD-L1 antibody or antigen binding fragment thereof described herein, e.g., 200 mg of the pembrolizumab antibody described in Table 2. In various embodiments, the anti-PD-1 antibody pembrolizumab is administered at 200 mg via intravenous infusion on Day 1 and then once every three weeks thereafter, and the anti-CD27 antibody is administered at 30 mg via intravenous infusion on Day 1 and then once every three weeks thereafter. In various embodiments, the anti-PD-1 antibody pembrolizumab is administered at 400 mg via intravenous infusion on Day 1 and then once every six weeks thereafter, and the anti-CD27 antibody is administered at 30 mg via intravenous infusion on Day 1 once every six weeks. In various embodiments, the anti-PD-1 antibody is co-formulated with the anti-CD27 antibody. An aspect of the invention provides a pharmaceutical composition comprising an isolated polynucleotide encoding the anti-CD27 antibody or antigen binding fragment thereof described herein (e.g., the antibody or antigen binding fragment thereof comprises at least one sequence described in Table 1), and a material or fluid (e.g., a pharmaceutically acceptable carrier buffer, and/or a diluent).

An aspect of the invention provides a kit comprising about 2 mg to about 700 mg of the anti-CD27 antibody or antigen binding fragment thereof described herein (e.g., the anti-CD27 antibody or antigen binding fragment thereof comprises at least one sequence described in Table 1), and instructions for use.

An aspect of the invention provides a kit comprising any of the pharmaceutical compositions comprising about 2 mg to about 700 mg of the anti-CD27 antibody or antigen binding fragment thereof described herein (e.g., the anti-CD27 antibody or antigen binding fragment thereof comprises at least one sequence described in Table 1), and instructions for use.

### BRIEF DESCRIPTION OF THE FIGURES

FIG. 1A and FIG. 1B are each waterfall plots of subjects with best percentage change from baseline in target lesion based on investigator assessment per RECIST v1.1 (evaluated in patients with measurable disease at baseline and ≥1 evaluable post-baseline target lesion imaging assessment/measurement (n=25 for antibody hCD27.131A monotherapy and n=19 for antibody hCD27.131A combination therapy with pembrolizumab)). Each bar represents an individual subject.

FIG. 2A and FIG. 2B are graphs showing change of baseline (%, percentage) in target lesions in subjects over time (weeks). The subjects had been treated with either antibody hCD27.131A monotherapy or a combination therapy including antibody hCD27.131A and pembrolizumab. The percentage change from baseline of target lesions was based on investigator assessment per RECIST v1.1. All patients had ≥1 post-baseline target lesion measurement; for patients who crossed over, only responses prior to cross over were included. Only patients who had first dosing on or before Mar. 29, 2019 were included.

FIG. 3A and FIG. 3B show compilation data plots of patients having solid tumors and the treatment duration and response based on investigator assessment per RECIST v1.1. Patients were either administered the specific doses of antibody hCD27.131A alone (FIG. 3A) or in a combination therapy with 200 mg pembrolizumab (FIG. 3B) as described in Example 1. The exposure and response duration results were based on investigator assessment per RECIST v1.1. For patients in FIG. 3A who crossed over, only responses prior to cross over were included. For patients in FIG. 3B who crossed over, only response post-crossover data were included.

FIG. 4 is an aggregated graph of combined study arms showing the percent of available CD27 receptors on representative CD3+CD4+ cells over time analyzed in samples from subjects administered antibody hCD27.131A either as a monotherapy or in combination with 200 mg pembrolizumab. The subjects were administered either 2 mg, 7 mg, 20 mg, 70 mg or 200 mg of hCD27.131A antibody. % Change from C1D1 Pre-Mean+/−SE.

FIGS. 5A-C are a series of aggregated graphs combined study arms showing changes in chemotactic cytokines MIP-1β (FIG. 5A), CD3+CD4+CD25+CD127-FOXP3+ regulatory T cells (FIG. 5B) and CD3+CD4+HLA-DR+ T-cells (FIG. 5C) over time analyzed in samples from subjects administered antibody hCD27.131A either as a monotherapy or in combination with 200 mg pembrolizumab. The subjects were administered either 2 mg, 7 mg, 20 mg, 70 mg or 200 mg of antibody hCD27.131A. Mean Fold Change from C1D1 Pre-+/−SE for MIP-1b. % Change from C1D1 Pre-Mean+/−SE for regulatory T cells and HLADR+ T-cells.

FIG. 6A and FIG. 6B are graphs showing serum concentrations of hCD27.131A over time following intravenous doses from 2 mg to 700 mg in cycle 1. The arithmetic mean of antibody hCD27.131A was plotted at nominal times.

FIG. 7 is a waterfall plot of subjects with best percentage change from baseline in target lesion based on investigator assessment per RECIST v1.1 after a combination treatment with escalating doses of antibody hCD27.131A with pembrolizumab (evaluated in patients with measurable disease at baseline and ≥1 evaluable post-baseline target lesion imaging assessment/measurement. [N=30 is the FAS population for combination therapy in initial treatment phase of dose escalation part and N=40 is the FAS population for combination therapy of crossover phase in dose escalation part.] Of the 40 patients 27 were patients who received combination therapy in the initial treatment phase of dose escalation part and 13 patients were patients who received combination therapy in crossover phase of dose escalation part. Each bar represents an individual subject.

FIG. 8 is a waterfall plot of subjects with best percentage change from baseline in target TNBC lesion based on investigator assessment per RECIST v1.1 after a combination treatment with escalating doses of antibody hCD27.131A with pembrolizumab (evaluated in patients with measurable disease at baseline and ≥1 evaluable post-baseline target lesion imaging assessment/measurement (n=22 for antibody hCD27.131A combination therapy with pembrolizumab; FAS population)). Each bar represents an individual subject.

### DETAILED DESCRIPTION

### Abbreviations

Throughout the detailed description and examples of the invention the following abbreviations will be used:

### Abbreviation/Term Definition

ADA Anti-drug antibody
ADCC Antibody-dependent cellular cytotoxicity
AE Adverse event
AJCC American Joint Committee on Cancer
ALT Alanine aminotransferase
ANC Absolute neutrophil count
aPTT Activated partial thromboplastin time
ASaT All-Subjects-as-Treated
ASCO American Society of Clinical Oncology
AST Aspartate aminotransferase
AUC Area under the curve
BCG Bacillus Calmette-Guérin
β-hCG β-human chorionic gonadotropin
CBR Clinical Benefit Rate
CDC Complement-dependent cytotoxicity
CDR Complementarity determining region in the immunoglobulin variable regions,
CHO Chinese hamster ovary
Cmax Maximum concentration
Cmin Minimum concentration
CNS Central nervous system
CR Complete response
CRC Colorectal cancer
CRF Case report form
CSF Colony-stimulating factor
CSR Clinical Study Report
CT Computed tomography
CTCAE Common Terminology Criteria for Adverse Events
CTLA-4 Cytotoxic T lymphocyte-associated antigen 4
D De-escalate to the next lower dose
DCR Disease Control Rate
DFS Disease free survival
DILI Drug-induced liver injury
DLT Dose-limiting toxicity
DNA Deoxyribonucleic acid
DOR Duration of Response
DSDR Durable Stable Disease Rate
DU Unacceptably toxic dose
ECG Electrocardiogram
ECI Event(s) of clinical interest
ECOG Eastern Cooperative Oncology Group
eCRF Electronic case report form
ELISA Enzyme-linked immunosorbant assay
EMA European Medicines Agency
FAS Full Analysis Set
FBR Future biomedical research
FDAA Food and Drug Administration Amendments Act
FFPE Formalin-fixed, paraffin-embedded
FR Antibody framework region: the immunoglobulin variable regions excluding the CDR regions.
FSH Follicle-stimulating hormone
GCP Good Clinical Practice
GFR Glomerular filtration rate
GGT Gamma glutamyl transferase
GI Gastrointestinal cancer
GU Genitourinary cancer
HBsAg Hepatitis B surface antigen
HCV Hepatitis C virus
HIV Human immunodeficiency virus
HRT Hormonal replacement therapy
HRP Horseradish peroxidase
IB Investigator's Brochure
IC50 concentration resulting in 50% inhibition
ICF Informed consent form
ICH International Conference on Harmonisation
iCPD iRECIST confirmed progressive disease
IEC Independent Ethics Committee
IFN interferon
Ig Immunoglobulin
IHC Immunohistochemistry
IL Interleukin
IM Intramuscular
INR International normalized ratio
irAE Immune-related adverse event
irRC Immune related response criteria
IRB Institutional Review Board
iRECIST Modified RECIST 1.1 for immune-based therapeutics
iSD iRECIST stable disease
iUPD iRECIST unconfirmed progressive disease
IV Intravenous
IVRS Interactive Voice Response System
IWRS Integrated Web Response System
Kabat An immunoglobulin alignment and numbering system pioneered by Elvin A. Kabat ((1991) Sequences of Proteins of Immunological Interest, 5th Ed. Public Health Service, National Institutes of Health, Bethesda, Md.)
LDH Lactate dehydrogenase
M1a single metastatic site)
M1b peritoneal or multiple metastatic sites
mAb or Mab or MAb Monoclonal antibody
MASCC Multinational Association of Supportive Care in Cancer
MRI Magnetic resonance imaging
mRNA Messenger ribonucleic acid
MSD Merck Sharp & Dohme Corp., a subsidiary of Merck & Co., Inc.
MSI Microsatellite instability
MTD Maximum tolerated dose
mTPI Modified toxicity probability interval
NCBI National Center for Biotechnology Information
NCI National Cancer Institute
NK Natural killer
NOAEL No observed adverse effect level
NSAID Non-steroidal anti-inflammatory drug
NSCLC Non-small cell lung cancer
ORR Objective response rate
OS Overall survival
OTC Over-the-counter
PBMC Peripheral blood mononuclear cell
PD Pharmacodynamic(s)
PD Progressive disease
PD-1 Programmed cell death 1
PD-L1 Programmed cell death ligand 1
PD-L2 Programmed cell death ligand 2
PFS Progression-free survival
PK Pharmacokinetic(s)
PP Per-Protocol
PR Partial response
PT Prothrombin time
PTT Partial thromboplastin time
Q2W One dose every two weeks
Q3W One dose every three weeks
Q6W One dose every six weeks
QD One dose per day
RECIST Response Evaluation Criteria in Solid Tumors
RECIST 1.1 Response Evaluation Criteria in Solid Tumors, version 1.1
RNA Ribonucleic acid
RP2D Recommended Phase 2 dose
SAE Serious adverse event
SAP Statistical Analysis Plan
SD Stable disease
SEB Staphylococcus Enterotoxin B
SGOT Serum glutamic oxaloacetic transaminase
SGPT Serum glutamic pyruvic transaminase
SNP Single nucleotide polymorphism
SoA Schedule of Activities
sSAP Supplementary Statistical Analysis Plan
STS soft tissue sarcoma
T½ or T½ half-life
TCR T cell receptor
TNBC Triple-negative breast cancer
TNF Tumor necrosis factor
TSH Thyroid-stimulating hormone
TT Tetanus toxoid
V region The segment of Ig chains which is variable in sequence between different antibodies. It extends to Kabat residue 107 in the light chain and 113 in the heavy chain.
VH Immunoglobulin heavy chain variable region
VK Immunoglobulin kappa light chain variable region
ULN Upper limit of normal
WOCBP Woman of childbearing potential

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Administration” as applied to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Administration to a cell encompasses contact of a reagent to the cell, as well as contact of a reagent to a fluid, where the fluid is in contact with the cell. “Administration” also mean in vitro and ex vivo administration, e.g., of a cell, by a reagent, diagnostic, binding compound, or by another cell.

A “biomarker” is an objectively measured indicator, compound or molecule that reflects the presence, or likely progression, or successful treatment of a particular condition. Biomarkers have long been used in drug development, and the discovery and validation of new efficacy biomarkers is expected to improve the predictive disease models, reduce the time and cost associated with drug development, and increase the success rate of translating experimental drugs into clinical therapeutics. In addition, biomarkers are valuable in early detection of disease development, changes in disease status, and effectiveness of behavioral modifications and therapeutics in disease control.

“Treat” or “treating” means to administer a therapeutic agent, such as a composition containing any of the antibodies or antigen-binding fragments thereof of the present invention, internally or externally to a subject or patient having one or more disease symptoms of cancer, or being suspected of having the disease, for which the agent has therapeutic activity. The agent is administered in an amount effective to alleviate one or more disease symptoms or disease indicia in the treated subject or population, whether by inducing the regression of or inhibiting the progression of such symptom(s) or indicia by any clinically measurable degree. The amount of a therapeutic agent that is effective to alleviate any particular disease symptom or disease indicia may vary according to factors such as the disease state, age, and weight of the patient, and the ability of the drug to elicit a desired response in the subject. Whether a disease symptom or a disease indicium has been alleviated can be assessed by any clinical measurement typically used by physicians or other skilled healthcare providers to assess the severity or progression status (over a period of time) of that symptom or that indicium. In various embodiments of the present invention, while the antibodies or antigen binding fragments and the pharmaceutical compositions may not be effective in preventing or alleviating the target disease symptom(s), target disease indicium, indicia or effects, or adverse effect(s) in every patient, it should alleviate such symptom(s), indicium, indicia, or effect(s) in a statistically significant number of patients (e.g., a number of patients over a period of time, such as days, months, or years) as determined by any statistical test known in the art such as the Student's t-test, the chi2-test, the U-test according to Mann and Whitney, the Kruskal-Wallis test (H-test), Jonckheere-Terpstra-test and the Wilcoxon-test. For example, in various embodiments disclosed herein, an anti-CD27 antibody or antigen binding fragment thereof (e.g., hCD27.131A) is administered to a subject or a patient who has active disease (e.g., cancer), and a therapeutically effective amount will result in a reduction of the measured symptom by some degree or percentage (e.g., least 5%, at least 10%, at least 20%, at least 30%, at least 40%, at least 50%, at least 60%, at least 70%, at least 80%, or at least 90%) over a period of time.

As used herein, the term “antibody” refers to any form of antibody that exhibits the desired biological activity. Thus, it is used in the broadest sense and specifically covers, but is not limited to, monoclonal antibodies (including full length monoclonal antibodies comprising two light chains and two heavy chains), polyclonal antibodies, multispecific antibodies (e.g., bispecific antibodies), humanized antibodies, fully human antibodies, and chimeric antibodies.

In general, the basic antibody structural unit comprises a tetramer. Each tetramer includes two identical pairs of polypeptide chains, each pair having one “light” (e.g., about 25 kDa) and one “heavy” chain (e.g., about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2^{nd} ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (referred to here as “CDRs”), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment”, “antigen binding fragment thereof”, “antigen binding fragment” or “antigen-binding fragment” refers to antigen-binding fragments of antibodies, i.e. antibody fragments that retain the ability to bind specifically to the antigen bound by the full-length antibody, e.g. fragments that retain one or more CDR regions. Examples of antigen-binding fragments include, but are not limited to, Fab, Fab′, F(ab′)₂, and Fv fragments; diabodies; linear antibodies; single-chain antibody molecules, e.g., sc-Fv; multispecific antibodies formed from antibody fragments.

A “Fab fragment” is comprised of one light chain and the C_{H}1 and variable regions of one heavy chain. The heavy chain of a Fab molecule cannot form a disulfide bond with another heavy chain molecule. A “Fab fragment” can be the product of papain cleavage of an antibody.

An “Fc” region contains two heavy chain fragments comprising the C_{H}3 and C_{H}2 domains of an antibody. The two heavy chain fragments are held together by two or more disulfide bonds and by hydrophobic interactions of the C_{H}3 domains.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody.

The “Fv region” comprises the variable regions from both the heavy and light chains, but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen-binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “bivalent antibody” comprises two antigen-binding sites. In some instances, the two binding sites have the same antigen specificities. However, bivalent antibodies may be bispecific (see below).

“Co-administration” as used herein for agents (e.g., anti-CD27 antibody or antigen binding fragment thereof, and an additional therapeutic agent, such as a PD-1 antibody or antigen binding fragment thereof) means that the agents are administered so as to have overlapping therapeutic activities, and not necessarily that the agents are administered simultaneously to the subject. The agents may or may not be in physical combination prior to administration. In an embodiment, the agents are administered to a subject simultaneously or at about the same time. For example, the anti-PD-1 antibody and anti-CD27 antibody or antigen binding fragments contained in separate vials, when in liquid solution, may be mixed into the same intravenous infusion bag or injection device, and administered simultaneously to the patient.

“Co-formulated” or “co-formulation” or “coformulation” or “coformulated” as used herein refers to at least two agents (e.g., different antibodies or antigen binding fragments) thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof described herein, for example, the anti-CD27 antibody or antigen binding fragments thereof described herein and the PD-1 antibody or antigen binding described herein.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

Typically, an antibody or antigen-binding fragment thereof as used herein is modified in some way retains at least 10% of its binding activity (when compared to the parental antibody) when that activity is expressed on a molar basis. Preferably, an antibody or antigen-binding fragment thereof of the invention retains at least 20%, 50%, 70%, 80%, 90%, 95% or 100% or more of the CD27 binding affinity as the parental antibody. It is also intended that an antibody or antigen-binding fragment thereof of the invention can include conservative or non-conservative amino acid substitutions (referred to as “conservative variants” or “function conserved variants” of the antibody) that do not substantially alter its biologic activity.

“Isolated” antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment thereof may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, a “chimeric antibody” is an antibody having the variable domain from a first antibody and the constant domain from a second antibody, where the first and second antibodies are from different species. (U.S. Pat. No. 4,816,567; and Morrison et al., (1984) Proc. Natl. Acad. Sci. USA 81: 6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g., mouse) antibody.

The present invention includes the use of anti-CD27 humanized antibodies and antigen-binding fragments thereof (e.g., rat or mouse antibodies that have been humanized) and methods of use thereof. The invention includes any humanized version of the hCD27.131A antibody. As used herein “131A antibody” and “hCD27.131A” are used interchangeably to refer to an antibody described herein, for example the antibody comprising the V_{H} region of SEQ ID NO.: 7 and the V_{L} region of SEQ ID NO.: 9.

As used herein, the term “humanized antibody” refers to forms of antibodies that contain sequences from both human and non-human (e.g., mouse or rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc). For more details about humanized antibodies, see, e.g., Jones et al., Nature, 321:522-525 (1986); Reichmann et al., Nature, 332:323-329 (1988); Presta, Curr. Op. Struct. Biol., 2:593-596 (1992); and Clark, Immunol. Today 21: 397-402 (2000).

As used herein, the term “framework” or “FR” residues refers to those variable domain residues other than the hypervariable region residues defined herein as CDR residues.

As used herein, the term “hypervariable region” refers to the amino acid residues of an antibody or antigen-binding fragment thereof that are responsible for antigen-binding. The hypervariable region comprises amino acid residues from a CDR (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure).

“Isolated nucleic acid molecule” or “isolated polynucleotide” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid or polynucleotide is “operably linked” when it is placed into afunctional relationship with another nucleic acid sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “germline sequence” refers to a sequence of unrearranged immunoglobulin DNA sequences. Any suitable source of unrearranged immunoglobulin sequences may be used. Human germline sequences may be obtained, for example, from JOINSOLVER germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

As used herein, “therapeutically effective amount” refers to an amount of the therapeutic agent (e.g., an anti-CD27 antibody or antigen binding fragment thereof such as hCD27.131A), which is effective as a monotherapy or in combination with the other active ingredient (e.g., PD-1 antibody or antigen binding fragment thereof), upon single or multiple dose administration (e.g., an infusion treatment over a period of time, such as 30 minutes, or a bolus and/or maintenance doses) to a subject, in inhibiting, delaying or reducing the growth of a cancer (e.g., a cancer characterized by presence of a solid tumor). Therapeutically effective is also intended to refer to an amount of the anti-CD27 antibody or antigen binding fragment thereof (when used as a monotherapy or in combination with the other active agent) that is suitable for therapeutic uses. In various embodiments, a therapeutically effective amount of an anti-CD27 antibody or antigen binding fragment thereof is the amount of the anti-CD27 antibody or antigen binding fragment thereof that is needed to treat a solid tumor cancer in combination with a therapeutic agent, for example an anti-PD-1 antibody such as pembrolizumab.

The therapeutically effective dose of any of the antibodies or antigen binding fragments thereof described herein (e.g., see Table 1) may be administered alone or in combination with an additional therapeutic agent to a cell, tissue, or subject, is effective to cause a measurable improvement in one or more symptoms of disease, for example cancer or the progression of cancer. A therapeutically effective dose may be an amount of the antibody or fragment thereof sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, a therapeutically effective dose may be combined with amounts of the active ingredients that result in the therapeutic effect, whether administered in combination, serially or simultaneously. An effective amount of a therapeutic may result in an improvement of a biomarker, measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “solid tumor cancer” is defined as a cancer disease wherein the malignant tumor is present at a distinct location and does not contain a cyst and/or liquid area. Solid tumors may be benign (not cancer), or malignant (cancer). Bladder cancer, breast cancer, colon and rectal cancer (colorectal cancer), endometrial cancer, kidney (renal cell) cancer, lung cancer, pancreatic cancer, prostate cancer, and thyroid cancer are non-limiting examples of different types of solid tumor cancers. Leukemia is an example of a cancer that is not a solid tumor cancer. See U.S. Pat. No. 10,451,626.

### Physical and Functional Properties of the Anti-CD27 Antibodies

The present invention provides anti-CD27 antibodies and antigen-binding fragments thereof having specified structural and functional features, and methods of use of the antibodies or antigen-binding fragments thereof in the treatment or prevention of disease (e.g., cancer). An “anti-CD27 antibody or antigen-binding fragment thereof of the present invention” includes: any antibody or antigen-binding fragment thereof that is discussed herein (e.g., hCD27.131A as shown below or humanized versions thereof disclosed in PCT publication number WO2018/058022) or a variant thereof (e.g., sequence variant or functional variant); any antibody or antigen-binding fragment thereof comprising any one or more of the (underlined and highlighted) CDRs set forth below in Table 1.

TABLE 1
The amino acid sequences of the antibody hCD27.131A
and antigen binding fragments thereof

| Antibody Feature/ | | |
|---|---|---|
| antigen binding | | |
| fragment | SEQ ID NO: | Amino acid sequence |
| hCD27.131A | 1 | NYGMN |
| H-CDR1 | | |
| hCD27.131A | 2 | WINTNTGEPTYAEEFKG |
| H-CDR2 | | |
| hCD27.131A | 3 | EGDAMDY |
| H-CDR3 | | |
| hCD27.131A | 4 | SASSSVSYMH |
| L-CDR1 | | |
| hCD27.131A | 5 | DTSKLAS |
| L-CDR2 | | |
| hCD27.131A | 6 | QQWNSYPFT |
| L-CDR3 | | |
| hCD27.131A | 7 | EIQLVQSGAEVKKPGASVKVSCKASGYTFTNYGMNWVKQA |
| variable heavy | | PGQGLKWMGWINTNTGEPTYAEEFKGRFTFTLDTSISTAY |
| region/domain | | MELSSLRSEDTAVYYCAREGDAMDYWGQGTTVTVSS |
| hCD27.131A | 8 | EIQLVQSGAEVKKPGASVKVSCKASGYTFTNYGMNWVKQA |
| heavy chain | | PGQGLKWMGWINTNTGEPTYAEEFKGRFTFTLDTSISTAY |
| | | MELSSLRSEDTAVYYCAREGDAMDYWGQGTTVTVSSASTK |
| | | GPSVFPLAPSSKSTSGGTAALGCLVKDYFPEPVTVSWNSG |
| | | ALTSGVHTFPAVLQSSGLYSLSSVVTVPSSSLGTQTYICN |
| | | VNHKPSNTKVDKKVEPKSCDKTHTCPPCPAPELLGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYKC |
| | | KVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKSL |
| | | SLSPGK |
| hCD27.131A | 9 | EIVLTQSPATLSLSPGERATLSCSASSSVSYMHWYQQKPG |
| variable light | | QAPKRWIYDTSKLASGVPARFSGSGSGTDYSLTISSLEPE |
| region/domain | | DFAVYYCQQWNSYPFTFGQGTKLEIK |
| hCD27.131A | 10 | EIVLTQSPATLSLSPGERATLSCSASSSVSYMHWYQQKPG |
| light chain | | QAPKRWIYDTSKLASGVPARFSGSGSGTDYSLTISSLEPE |
| | | DFAVYYCQQWNSYPFTFGQGTKLEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |

TABLE 2
Exemplary PD-1 Antibody Sequence for Pembrolizumab

| | | |
|---|---|---|
| Antibody Feature/ | | |
| antigen binding | | |
| fragment | Amino Acid Sequence | SEQ ID NO. |

Pembrolizumab Light Chain

| | | |
|---|---|---|
| CDR1 | RASKGVSTSGYSYLH | 11 |
| CDR2 | LASYLES | 12 |
| CDR3 | QHSRDLPLT | 13 |
| Variable Region | EIVLTQSPATLSLSPGERATLSCRASKGVSTSGYSYLHWYQQKPGQA | 14 |
| | PRLLIYLASYLESGVPARFSGSGSGTDFTLTISSLEPEDFAVYYCQH | |
| | SRDLPLTFGGGTKVEIK | |
| Light Chain | EIVLTQSPATLSLSPGERATLSCRASKGVSTSGYSYLHWYQQKPGQA | 15 |
| | PRLLIYLASYLESGVPARFSGSGSGTDFTLTISSLEPEDFAVYYCQH | |
| | SRDLPLTFGGGTKVEIKRTVAAPSVFIFPPSDEQLKSGTASVVCLLN | |
| | NFYPREAKVQWKVDNALQSGNSQESVTEQDSKDSTYSLSSTLTLSKA | |
| | DYEKHKVYACEVTHQGLSSPVTKSFNRGEC | |

Pembrolizumab Heavy Chain

| | | |
|---|---|---|
| CDR1 | NYYMY | 16 |
| CDR2 | GINPSNGGTNFNEKFKN | 17 |
| CDR3 | RDYRFDMGFDY | 18 |
| Variable Region | QVQLVQSGVEVKKPGASVKVSCKASGYTFTNYYMYWVRQAPGQGLEW | 19 |
| | MGGINPSNGGTNFNEKFKNRVTLTTDSSTTTAYMELKSLQFDDTAVY | |
| | YCARRDYRFDMGFDYWGQGTTVTVSS | |
| Heavy Chain | QVQLVQSGVEVKKPGASVKVSCKASGYTFTNYYMYWVRQAPGQGLEW | 20 |
| | MGGINPSNGGTNFNEKFKNRVTLTTDSSTTTAYMELKSLQFDDTAVY | |
| | YCARRDYRFDMGFDYWGQGTTVTVSSASTKGPSVFPLAPCSRSTSES | |
| | TAALGCLVKDYFPEPVTVSWNSGALTSGVHTFPAVLQSSGLYSLSSV | |
| | VTVPSSSLGTKTYTCNVDHKPSNTKVDKRVESKYGPPCPPCPAPEFL | |
| | GGPSVFLFPPKPKDTLMISRTPEVTCVVVDVSQEDPEVQFNWYVDGV | |
| | EVHNAKTKPREEQFNSTYRVVSVLTVLHQDWLNGKEYKCKVSNKGLP | |
| | SSIEKTISKAKGQPREPQVYT | |

As stated above, antibodies and fragments thereof that bind to the same epitope as any of the anti-CD27 antibodies or antigen-binding fragments thereof of the present invention also may be used as part of the present invention. In one embodiment, the invention provides an antibody or antigen binding fragment thereof that binds to the same epitope of human CD27 as an antibody or antigen binding fragment thereof described herein, for example, an antibody or antigen binding fragment thereof comprising the variable heavy chain comprising the amino acid sequence of SEQ ID NO.: 7 and the variable light chain comprising the amino acid sequence of SEQ ID NO.: 9. In another embodiment, the invention provides an antibody or antigen binding fragment thereof that binds to the same epitope of human CD27 as an antibody comprising the variable heavy chain comprising the amino acid sequence of SEQ ID NO.: 7 and the variable light chain comprising the amino acid sequence of SEQ ID NO.: 9. There are several methods available for mapping antibody epitopes on target antigens, including: H/D-Ex Mass spec, X-ray crystallography, pepscan analysis, alanine scanning, hydroxyl radical footprinting and site directed mutagenesis. For example, HDX (Hydrogen Deuterium Exchange) coupled with proteolysis and mass spectrometry can be used to determine the epitope of an antibody on a specific antigen Y. HDX-MS relies on the accurate measurement and comparison of the degree of deuterium incorporation by an antigen when incubated in D20 on its own and in presence of its antibody at various time intervals. Deuterium is exchanged with hydrogen on the amide backbone of the proteins in exposed areas whereas regions of the antigen bound to the antibody will be protected and will show less or no exchange after analysis by LC-MS/MS of proteolytic fragments. In one embodiment, the epitope is determined by solving the X-ray crystal structure of a complex between CD27 or fragment thereof and an anti-CD27 antibody or fragment thereof and identifying one or more CD27 residues within 4 Å of the anti-CD27 antibody residues. In another embodiment, the epitope includes for example, CD27 residues that have van der Waals, polar interaction, salt bridge or hydrogen bond contact with the anti-CD27 antibody residues. In another embodiment, the epitope is determined by mutagenesis (for example Alanine scanning) of CD27 residues and analyzing the loss of binding to the anti-CD27 antibody as a result of the mutagenesis.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 3.

TABLE 3
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the antibodies are also contemplated as being useful in the methods and uses described herein. “Function-conservative variants,” as used herein, refers to antibodies or fragments thereof in which one or more amino acid residues have been changed without altering a desired property, such as an antigen affinity and/or specificity.

Such variants include, but are not limited to, replacement of an amino acid with one having similar properties, such as the conservative amino acid substitutions of Table 3. Also provided are isolated polypeptides comprising the V_{L} domains of the anti-CD27 antibodies of the invention (e.g., SEQ ID NO.: 9), and isolated polypeptides comprising the V_{H} domains of the anti-CD27 antibodies of the invention (e.g., SEQ ID NO.: 7) having up to 0, 1, 2, 3, 4, 5, 6, 7, 8, 9, 10 or more amino acid substitutions.

In another embodiment, an antibody or antigen-binding fragment thereof that binds CD27 and has V_{L} domains and V_{H} domains with at least 99% 98%, 97%, 96%, 95%, 90%, 85%, 80% or 75% sequence identity to one or more of the V_{L} domains or V_{H} domains described herein and exhibits specific binding to CD27 may also be utilized in the methods and uses described herein. In another embodiment the binding antibody or antigen-binding fragment thereof of the present invention comprises V_{L} and V_{H} domains (with and without signal sequence) having up to 1, 2, 3, 4, 5, 6, 7, 8, 9, 10, 11, 12, 13, 14, 15, 16, 17, 18, 19, 20, 21, 22, 23, 24, 25 or more amino acid substitutions, and exhibits specific binding to CD27.

### Polynucleotides and Polypeptides

The present invention further comprises methods and uses for treating cancer that comprise polynucleotides encoding any of the polypeptides or immunoglobulin chains of anti-CD27 antibodies and antigen-binding fragments thereof of the invention. For example, the present invention includes polynucleotides encoding an anti-CD27 antibody or antigen-binding fragment thereof having the amino acids described in Table 1.

In one embodiment, an isolated polynucleotide, for example DNA, encoding the polypeptide chains of the isolated antibodies or antigen-binding fragments thereof set forth herein is provided. In one embodiment, the isolated polynucleotide encodes an antibody or antigen-binding fragment thereof comprising at least one mature immunoglobulin light chain variable (V_{L}) domain according to the invention and/or at least one mature immunoglobulin heavy chain variable (V_{H}) domain according to the invention. In some embodiments the isolated polynucleotide encodes both a light chain and a heavy chain on a single polynucleotide molecule, and in other embodiments the light and heavy chains are encoded on separate polynucleotide molecules. In another embodiment the polynucleotide further encodes a signal sequence.

In one embodiment, the invention comprises methods and uses comprising an isolated polynucleotide encoding a V_{H} domain or an antigen-binding fragment thereof comprising CDR-H1 (SEQ ID NO.: 1), CDR-H2 (SEQ ID NO.: 2) and CDR-H3 (SEQ ID NO.: 3). In one embodiment, the invention comprises methods and uses comprising an isolated polynucleotide encoding a V_{L} domain or an antigen-binding fragment thereof comprising CDR-L1 (SEQ ID NO.: 4), CDR-L2 (SEQ ID NO.: 5) and CDR-L3 (SEQ ID NO.: 6). In one embodiment, the invention comprises methods and uses for treating cancer comprising an isolated polynucleotide encoding the V_{H} domain methods and uses for treating cancer comprising the amino acid sequence of SEQ ID NO.: 7. In one embodiment, the invention methods and uses for treating cancer comprising an isolated polynucleotide encoding the V_{L} domain comprising the amino acid sequence of SEQ ID NO.: 9. In one embodiment, the invention comprises methods and uses for treating cancer comprising an isolated polynucleotide encoding the heavy chain comprising the amino acid sequence of SEQ ID NO.: 8. In one embodiment, the invention comprises an isolated polynucleotide encoding the light chain comprising the amino acid sequence of SEQ ID NO.: 10.

This present invention also provides methods and uses for treating cancer comprising vectors, e.g., expression vectors, such as plasmids, comprising the isolated polynucleotides of the invention, wherein the polynucleotide is operably linked to control sequences that are recognized by a host cell when the host cell is transfected with the vector. Also provided are host cells comprising a vector of the present invention and methods for producing the antibody or antigen-binding fragment thereof or polypeptide disclosed herein (e.g., Table 1) comprising culturing a host cell harboring an expression vector or a nucleic acid encoding the immunoglobulin chains of the antibody or antigen-binding fragment thereof in culture medium, and isolating the antigen or antigen-binding fragment thereof from the host cell or culture medium.

Also included in the present invention are polypeptides, e.g., immunoglobulin polypeptides, comprising amino acid sequences that are at least about 75% identical, 80% identical, more preferably at least about 90% identical and most preferably at least about 95% identical (e.g., 95%, 96%, 97%, 98%, 99%, 100%) to the amino acid sequences of the

antibodies provided herein when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Sequence identity refers to the degree to which the amino acids of two polypeptides are the same at equivalent positions when the two sequences are optimally aligned.

The following references relate to BLAST algorithms often used for sequence analysis: BLAST ALGORITHMS: Altschul et al. (2005) FEBS J. 272(20): 5101-5109;

Altschul, S. F., et al., (1990)J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of Multiple distinct local alignments.” in Theoretical and Computational Methods in Genome Research (S. Suhai, ed.), (1997) pp. 1-14, Plenum, New York.

Binding Affinity

By way of example, and not limitation, methods and uses for treating cancer comprise administering the antibodies and antigen-binding fragments thereof disclosed herein (e.g., comprising an amino acid sequence of SEQ ID NO.: 19 or SEQ ID NO.: 20) that may bind human CD27 or CD27A59T) with a bivalent K_{D} value of 10×10⁻⁹M or lower as determined by surface plasmon resonance (e.g., BIACORE) or a similar technique (e.g., KinExa or OCTET) as measured with a human CD27-Fc fusion protein or human CD27A59T-Fc fusion protein. In one embodiment, methods and uses for treating cancer comprise administering the antibodies and antigen-binding fragments thereof disclosed herein, wherein the antibodies or antigen binding fragments may bind human CD27 or CD27A59T with a bivalent K_{D} value of about 5×10⁻⁹ to about 10×10⁻⁹M as determined by surface plasmon resonance (e.g., BIACORE) or a similar technique (e.g. KinExa or OCTET) as measured using a CD27 protein or peptide, for example with a human CD27-Fc fusion protein or human CD27A59T-Fc fusion protein.

Immune Cell Activation

In some embodiments, methods and uses for treating cancer comprise administering an antibodies or antigen binding fragment thereof that increase the activity of an immune cell. The increase of the activity of an immune cell can be detected using any method known in the art. In one embodiment, the increase in activity of an immune cell can be detected by measuring the proliferation of the immune cell.

Methods of Making Antibodies and Antigen-Binding Fragments Thereof

Methods for making an anti-CD27 antibody or antigen-binding fragment thereof (e.g., Table 1) are described herein. For example, methods comprising culturing a hybridoma cell that expresses the antibody or fragment thereof under conditions favorable to such expression and, optionally, isolating the antibody or fragment thereof from the hybridoma and/or the growth medium (e.g., cell culture medium).

The anti-CD27 antibodies disclosed herein may also be produced recombinantly (e.g., in an E. coli/T7 expression system, a mammalian cell expression system or a lower eukaryote expression system). Nucleic acids encoding the antibody immunoglobulin molecules of the invention (e.g., V_{H} or V_{L}) may be inserted into a pET-based plasmid and expressed in the E. coli/T7 system. For example, methods for expressing an antibody or antigen-binding fragment thereof or immunoglobulin chain thereof in a host cell (e.g., bacterial host cell such as E. coli such as BL21 or BL21DE3) are described herein. In various embodiments, the methods comprise expressing T7 RNA polymerase in the cell which also includes a polynucleotide encoding an immunoglobulin chain that is operably linked to a T7 promoter. For example, a bacterial host cell, such as a E. coli, includes a polynucleotide encoding the T7 RNA polymerase gene operably linked to a lac promoter and expression of the polymerase and the chain is induced by incubation of the host cell with isopropyl-beta-D-thiogalactopyranoside (IPTG).

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Described herein are recombinant methods for making an anti-CD27 antibody or antigen-binding fragment thereof of the present invention, or an immunoglobulin chain thereof, comprising introducing a polynucleotide encoding one or more immunoglobulin chains of the antibody or fragment thereof (e.g., heavy and/or light immunoglobulin chain); culturing the host cell (e.g., CHO or Pichia or Pichia pastoris) under condition favorable to such expression and, optionally, isolating the antibody or fragment thereof or chain from the host cell and/or medium in which the host cell is grown. Anti-CD27 antibodies can also be synthesized by any of the methods set forth in U.S. Pat. No. 6,331,415.

Eukaryotic and prokaryotic host cells, including mammalian cells as hosts for expression of the antibodies or fragments thereof or immunoglobulin chains disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody or fragment thereof or chain in the host cells or secretion of the into the culture medium in which the host cells are grown.

Antibodies and antigen-binding fragments thereof and immunoglobulin chains can be recovered from the culture medium using standard protein purification methods. Further, expression of antibodies and antigen-binding fragments thereof and immunoglobulin chains (or other moieties therefrom) are described herein. The expression for examples is achieved from production cell lines that can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4. The mammalian host cells (e.g., CHO) may lack a glutamine synthetase gene and are grown in the absence of glutamine in the medium wherein, however, the polynucleotide encoding the immunoglobulin chain comprises a glutamine synthetase gene which complements the lack of the gene in the host cell.

In general, glycoproteins produced in a particular cell line or transgenic animal will have a glycosylation pattern that is characteristic for glycoproteins produced in the cell line or transgenic animal. Therefore, the particular glycosylation pattern of an antibody will depend on the particular cell line or transgenic animal used to produce the antibody. However, all antibodies encoded by the nucleic acid molecules provided herein, or comprising the amino acid sequences provided herein, comprise the instant invention, independent of the glycosylation pattern the antibodies may have. Similarly, in particular embodiments, antibodies with a glycosylation pattern comprising only non-fucosylated N-glycans may be advantageous, because these antibodies have been shown to typically exhibit more potent efficacy than their fucosylated counterparts both in vitro and in vivo (See for example, Shinkawa et al., J. Biol. Chem. 278: 3466-3473 (2003); U.S. Pat. Nos. 6,946,292 and 7,214,775). These antibodies with non-fucosylated N-glycans are not likely to be immunogenic because their carbohydrate structures are a normal component of the population that exists in human serum IgG.

Bispecific and bifunctional antibodies and antigen-binding fragments thereof having a binding specificity for CD27 and another antigen such as, for example, PD-1 or PD-L1, and methods of use thereof are described herein. In an embodiment of the invention, the anti-CD27 chains comprise any one of the VH/VL sequences described in Table 1, for example the anti-PD1 chains comprise the amino acid sequence of SEQ ID NOs: 14 and 19 or of SEQ ID NOs: 15 and 20 (or an antigen binding fragment thereof of any of said sequences) as shown in Table 1 and/or Table 2. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai, et al., (1990) Clin. Exp. Immunol. 79: 315-321, Kostelny, et al., (1992) J Immunol. 148:1547-1553. In addition, bispecific antibodies may be formed as “diabodies” (Holliger, et al., (1993) PNAS USA 90:6444-6448) or as “Janusins” (Traunecker, et al., (1991) EMBO 110:3655-3659 and Traunecker, et al., (1992) Int. J Cancer Suppl. 7:51-52).

The present invention further includes methods and uses comprising administering anti-CD27 antigen-binding fragments thereof of the anti-CD27 antibodies disclosed herein. The antibody fragments thereof include F(ab)₂ fragments, which may be produced by enzymatic cleavage of an IgG by, for example, pepsin. Fab fragments may be produced by, for example, reduction of F(ab)₂ with dithiothreitol or mercaptoethylamine.

Immunoglobulins may be assigned to different classes depending on the amino acid sequences of the constant domain of their heavy chains. In some embodiments, different constant domains may be appended to humanized V_{L} and V_{H} regions derived from the CDRs provided herein. There are at least five major classes of immunoglobulins: IgA, IgD, IgE, IgG and IgM, and several of these may be further divided into subclasses (isotypes), e.g. IgG1, IgG2, IgG3 and IgG4; IgA1 and IgA2. Antibodies and antigen-binding fragments of any of these classes or subclasses of antibodies may be utilized and administered.

The antibody or antigen-binding fragment thereof in various embodiments comprises a heavy chain constant region, e.g. a human constant region, such as γ1, γ2, γ3, or γ4 human heavy chain constant region or a variant thereof. In another embodiment, the antibody or antigen-binding fragment thereof comprises a light chain constant region, e.g. a human light chain constant region, such as lambda or kappa human light chain region or variant thereof. By way of example, and not limitation the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38: 1-8, 2001).

The methods and uses described herein in various embodiments comprise administering an anti-CD27 antibody or antigen-binding fragment thereof that comprises a heavy chain constant region of the IgG1 subtype.

Antibody Engineering

Further included are embodiments in which the anti-CD27 antibodies and antigen-binding fragments thereof are engineered antibodies to include modifications to framework residues within the variable domains of the monoclonal antibody, e.g. to improve the properties of the antibody or fragment thereof. Typically, such framework modifications are made to decrease the immunogenicity of the antibody or fragment thereof. This is usually accomplished by replacing non-CDR residues in the variable domains (i.e., framework residues) in a parental (e.g., rodent) antibody or fragment thereof with analogous residues from the immune repertoire of the species in which the antibody is to be used, e.g., human residues in the case of human therapeutics. Such an antibody or fragment thereof is referred to as a “humanized” antibody or fragment thereof. In some embodiments, it is desirable to increase the affinity, or alter the specificity of an engineered (e.g., humanized) antibody. One approach is to “backmutate” one or more framework residues to the corresponding germline sequence. More specifically, an antibody or fragment thereof that has undergone somatic mutation can contain framework residues that differ from the germline sequence from which the antibody is derived. Such residues can be identified by comparing the antibody or fragment framework sequences to the germline sequences from which the antibody or fragment thereof is derived. Another approach is to revert to the original parental (e.g., rodent) residue at one or more positions of the engineered (e.g., humanized) antibody, e.g. to restore binding affinity that may have been lost in the process of replacing the framework residues. (See, e.g., U.S. Pat. Nos. 5,693,762, 5,585,089 and 5,530,101.)

In certain embodiments, the anti-CD27 antibodies and antigen-binding fragments thereof are engineered (e.g., humanized) to include modifications in the framework and/or CDRs to improve their properties. Such engineered changes can be based on molecular modeling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, Md.; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al., (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice, the potential antigen binding regions based on model differ from the conventional “CDR”s or “hyper variable” loops. Commercial scientific software such as MOE (Chemical Computing Group) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases, the certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689.

In particular embodiments, it will be desirable to change certain amino acids containing exposed side-chains to another amino acid residue in order to provide for greater chemical stability of the final antibody, so as to avoid deamidation or isomerization. The deamidation of asparagine may occur on NG, DG, NG, NS, NA, NT, QG or QS sequences and result in the creation of an isoaspartic acid residue that introduces a kink into the polypeptide chain and decreases its stability (isoaspartic acid effect). Isomerization can occur at DG, DS, DA or DT sequences. In certain embodiments, the antibodies do not contain deamidation or asparagine isomerism sites.

For example, an asparagine (Asn) residue may be changed to Gln or Ala to reduce the potential for formation of isoaspartate at any Asn-Gly sequences, particularly within a CDR.

A similar problem may occur at an Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734.

In one embodiment, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe or other amino acids in order to reduce the possibility that the methionine sulfur would oxidize, which could reduce antigen-binding affinity and also contribute to molecular heterogeneity in the final antibody preparation. Id. Additionally, in order to prevent or minimize potential scissile Asn-Pro peptide bonds, it may be desirable to alter any Asn-Pro combinations found in a CDR to Gln-Pro, Ala-Pro, or Asn-Ala. Antibodies with such substitutions are subsequently screened to ensure that the substitutions do not decrease the affinity or specificity of the antibody for CD27, or other desired biological activity to unacceptable levels. Exemplary variants for the CDRs is shown in Table 4.

TABLE 4
Exemplary stabilizing CDR variants

| CDR Residue | Stabilizing Variant Sequence |
|---|---|
| Asn-Gly (N-G) | Gln-Gly, Ala-Gly, or Asn-Ala |
| | (Q-G), (A-G), or (N-A) |
| Asp-Gly (D-G) | Glu-Gly, Ala-Gly or Asp-Ala |
| | (E-G), (A-G), or (D-A) |
| Met (M) | Lys, Leu, Ala, or Phe |
| | (K), (L), (A), or (F) |
| Asn (N) | Gln or Ala (Q) or (A) |
| Asn-Pro (N-P) | Gln-Pro, Ala-Pro, or Asn-Ala |
| | (Q-P), (A-P), or (N-A) |

Antibody Engineering of the Fc Region

The antibodies (e.g., humanized antibodies) and antigen-binding fragments thereof disclosed herein (e.g., antibody hCD27.131A) can also be engineered to include modifications within the Fc region, typically to alter one or more properties of the antibody, such as serum half-life, complement fixation, Fc receptor binding, and/or effector function (e.g., antigen-dependent cellular cytotoxicity). Furthermore, the antibodies and antigen-binding fragments thereof disclosed herein (e.g., antibody 131A and humanized versions thereof) can be chemically modified (e.g., one or more chemical moieties can be attached to the antibody) or be modified to alter its glycosylation, again to alter one or more properties of the antibody or fragment thereof. Each of these embodiments is described in further detail below. The numbering of residues in the Fc region is that of the EU index of Kabat. The antibodies and antigen-binding fragments thereof disclosed herein (e.g., antibody hCD27.131A and humanized versions thereof) also include antibodies and fragments with modified (or blocked) Fc regions to provide altered effector functions. See, e.g., U.S. Pat. No. 5,624,821; and PCT publications numbers WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc regions. Changes to the Fc can also alter the half-life of antibodies in therapeutic antibodies, enabling less frequent dosing and thus increased convenience and decreased use of material. See Presta (2005) J. Allergy Clin. Immunol. 116:731 at 734-35.

In one embodiment, the antibody or antigen-binding fragment thereof described herein (e.g., antibody hCD27.131A and humanized versions thereof) is an IgG4 isotype antibody or fragment thereof comprising a Serine to Proline mutation at a position corresponding to position 228 (S228P; EU index) in the hinge region of the heavy chain constant region. This mutation has been reported to abolish the heterogeneity of inter-heavy chain disulfide bridges in the hinge region (Angal et al. supra; position 241 is based on the Kabat numbering system).

In one embodiment of the invention, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody.

In another embodiment, the Fc hinge region of an antibody or antigen-binding fragment thereof (e.g., antibody hCD27.131A and humanized versions thereof) is mutated to decrease the biological half-life of the antibody or fragment thereof. More specifically, one or more amino acid mutations are introduced into the CH2-CH3 domain interface region of the Fc-hinge fragment such that the antibody or fragment thereof has impaired Staphylococcyl protein A (SpA) binding relative to native Fc-hinge domain SpA binding. This approach is described in further detail in U.S. Pat. No. 6,165,745.

In another embodiment, the antibody or antigen-binding fragment thereof (e.g., antibody hCD27.131A and humanized versions thereof) is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other embodiments, the Fc region is altered by replacing at least one amino acid residue with a different amino acid residue to alter the effector function(s) of the antibody or antigen-binding fragment thereof. For example, one or more amino acids selected from amino acid residues 234, 235, 236, 237, 297, 318, 320 and 322 can be replaced with a different amino acid residue such that the antibody has an altered affinity for an effector ligand and retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551.

In another example, one or more amino acid residues within amino acid positions 231 and 239 are altered to thereby alter the ability of the antibody to fix complement. This approach is described further in PCT Publication WO 94/29351.

In yet another example, the Fc region is modified to decrease the ability of the antibody or antigen-binding fragment thereof (e.g., antibody hCD27.131A and humanized versions thereof) to mediate antibody dependent cellular cytotoxicity (ADCC) and/or to decrease the affinity of the antibody or fragment thereof for an Fcγ receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072. Moreover, the binding sites on human IgG1 for FcγR1, FcγRII, FcγRIII and FcRn have been mapped and variants with improved binding have been described (see Shields et al. (2001) J. Biol. Chem. 276:6591-6604).

In one embodiment, the Fc region is modified to decrease the ability of the antibody (e.g., antibody hCD27.131A) to mediate effector function and/or to increase anti-inflammatory properties by modifying residues 243 and 264. In one embodiment, the Fc region of the antibody or fragment thereof is modified by changing the residues at positions 243 and 264 to alanine. In one embodiment, the Fc region is modified to decrease the ability of the antibody or fragment thereof to mediate effector function and/or to increase anti-inflammatory properties by modifying residues 243, 264, 267 and 328.

Altered Effector Function

In some embodiments, the Fc region of an anti-CD27 antibody is modified to increase or reduce the ability of the antibody or antigen-binding fragment thereof to mediate effector function and/or to increase/decrease their binding to the Fc gamma receptors (FcγRs).

The term “Effector Function” as used herein is meant to refer to one or more of antibody dependent dell mediated cytotoxic activity (ADCC), complement-dependent cytotoxic activity (CDC) mediated responses, Fc-mediated phagocytosis or antibody dependent cellular phagocytosis (ADCP) and antibody recycling via the FcRn receptor.

The interaction between the constant region of an antigen binding protein and various Fc receptors (FcR) including Fc gammaRI (CD64), Fc gammaRII (CD32) and Fc gammaRIII (CD16) is believed to mediate the effector functions, such as ADCC and CDC, of the antigen binding protein. The Fc receptor is also important for antibody cross-linking, which can be important for anti-tumor immunity.

Effector function can be measured in a number of ways including for example via binding of the Fc gammaRIII to Natural Killer cells or via Fc gammaRI to monocytes/macrophages to measure for ADCC effector function. For example, an antigen binding protein of the present invention can be assessed for ADCC effector function in a Natural Killer cell assay. Examples of such assays can be found in Shields et al., 2001 J. Biol. Chem., Vol. 276, p 6591-6604; Chappel et al., 1993 J. Biol. Chem., Vol 268, p 25124-25131; Lazar et al, 2006 PNAS, 103; 4005-4010.

Human IgG1 constant regions containing specific mutations or altered glycosylation on residue Asn297 have been shown to reduce binding to Fc receptors. In other cases, mutations have also been shown to enhance ADCC and CDC (Lazar et al., PNAS 2006, 103; 4005-4010; Shields et al. J Biol Chem 2001, 276; 6591-6604; Nechansky et al., Mol Immunol, 2007, 44; 1815-1817).

In one embodiment of the present invention, such mutations are in one or more of positions selected from 239, 332 and 330 (IgG1), or the equivalent positions in other IgG isotypes. Examples of suitable mutations are S239D and I332E and A330L. In one embodiment, the antigen binding protein of the invention herein described is mutated at positions 239 and 332, for example S239D and I332E or in a further embodiment it is mutated at three or more positions selected from 239 and 332 and 330, for example S239D and I332E and A330L. (EU index numbering).

In an alternative embodiment, there is provided an antibody or antigen binding fragment thereof comprising a heavy chain constant region with an altered glycosylation profile such that the antigen binding protein has enhanced effector function. For example, wherein the antibody has enhanced ADCC or enhanced CDC or wherein it has both enhanced ADCC and CDC effector function. Examples of suitable methodologies to produce antigen binding proteins with an altered glycosylation profile are described in PCT publication numbers WO2003011878 and WO2006014679 and European patent application number EP1229125.

In a further aspect, the present invention provides “non-fucosylated” or “afucosylated” antibodies. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of Fc gammaRIIIa binding capacity.

Methods for producing the anti-CD27 antibody or antigen binding fragment thereof are also described, for example comprising the steps of: a) culturing a recombinant host cell comprising an expression vector comprising the isolated nucleic acid as described herein, wherein the recombinant host cell does not comprise an alpha-1,6-fucosyltransferase; and b) recovering the antigen binding protein. The recombinant host cell may not normally contain a gene encoding an alpha-1,6-fucosyltransferase (for example yeast host cells such as Pichia sp.) or may have been genetically modified to inactivate an alpha-1,6-fucosyltransferase. Recombinant host cells which have been genetically modified to inactivate the FUT8 gene encoding an alpha-1,6-fucosyltransferase are available. See, e.g., the POTELLIGENT™ technology system available from BioWa, Inc. (Princeton, N.J.) in which CHOK1SV cells lacking a functional copy of the FUT8 gene produce monoclonal antibodies having enhanced antibody dependent cell mediated cytotoxicity (ADCC) activity that is increased relative to an identical monoclonal antibody produced in a cell with a functional FUT8 gene. Aspects of the POTELLIGENT™ technology system are described in U.S. Pat. Nos. 7,214,775, 6,946,292, WO0061739 and WO0231240. Those of ordinary skill in the art will also recognize other appropriate systems.

It will be apparent to those skilled in the art that such modifications may not only be used alone but may be used in combination with each other in order to further enhance or decrease effector function.

Production of Antibodies with Modified Glycosylation

In still another embodiment, the antibodies or antigen-binding fragments (e.g., antibody hCD27.131A and variant versions thereof) comprise a particular glycosylation pattern. For example, an afucosylated or an aglycosylated antibody or fragment thereof can be made (i.e., the antibody lacks fucose or glycosylation, respectively). The glycosylation pattern of an antibody or fragment thereof may be altered to, for example, increase the affinity or avidity of the antibody or fragment thereof for a CD27 antigen. Such modifications can be accomplished by, for example, altering one or more of the glycosylation sites within the antibody or fragment thereof sequence. For example, one or more amino acid substitutions can be made that result in removal of one or more of the variable region framework glycosylation sites to thereby eliminate glycosylation at that site. Such aglycosylation may increase the affinity or avidity of the antibody or fragment thereof for antigen. See, e.g., U.S. Pat. Nos. 5,714,350 and 6,350,861.

Antibodies and antigen-binding fragments (e.g., antibody hCD27.131A and humanized versions thereof) may further include those produced in lower eukaryote host cells, in particular fungal host cells such as yeast and filamentous fungi have been genetically engineered to produce glycoproteins that have mammalian- or human-like glycosylation patterns (See for example, Choi et al., (2003) Proc. Natl. Acad. Sci. 100: 5022-5027; Hamilton et al., (2003) Science 301: 1244-1246; Hamilton et al., (2006) Science 313: 1441-1443; Nett et al., Yeast 28(3):237-52 (2011); Hamilton et al., Curr Opin Biotechnol. October; 18(5):387-92 (2007)). A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures (See for example, Li et al., (2006) Nat. Biotechnol. 24:210-215).

In particular embodiments, the antibodies and antigen-binding fragments thereof (e.g., antibody hCD27.131A) further include those produced in lower eukaryotic host cells and which comprise fucosylated and non-fucosylated hybrid and complex N-glycans, including bisected and multiantennary species, including but not limited to N-glycans such as GlcNAc₍₁₋₄₎Man₃GlcNAc₂; Gal₍₁₋₄₎GlcNAc₍₁₋₄₎Man₃GlcNAc₂; NANA₍₁₋₄₎Gal₍₁₋₄₎GlcNAc₍₁₋₄₎Man₃GlcNAc₂.

In particular embodiments, the antibodies and antigen-binding fragments thereof (e.g., antibody hCD27.131A) may comprise antibodies or fragments having at least one hybrid N-glycan selected from the group consisting of GlcNAcMan₅GlcNAc₂; GalGlcNAcMan₅GlcNAc₂; and NANAGalGlcNAcMan₅GlcNAc₂. In particular aspects, the hybrid N-glycan is the predominant N-glycan species in the composition.

In particular embodiments, the antibodies and antigen-binding fragments thereof (e.g., antibody hCD27.131A and humanized versions thereof) comprise antibodies and fragments having at least one complex N-glycan selected from the group consisting of GlcNAcMan₃GlcNAc₂; GalGlcNAcMan₃GlcNAc₂; NANAGalGlcNAcMan₃GlcNAc₂; GlcNAc₂Man₃GlcNAc₂; GalGlcNAc₂Man₃GlcNAc₂; Gal₂GlcNAc₂Man₃GlcNAc₂; NANAGal₂GlcNAc₂Man₃GlcNAc₂; and NANA₂Gal₂GlcNAc₂Man₃GlcNAc₂. In particular aspects, the complex N-glycan are the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In one embodiment, the antibody and antigen binding fragments thereof provided herein comprise complex N-glycans, wherein at least 50%, 60%, 70%, 80%, 90%, 95%, 97%, 98%, 99%, or 100% of the complex N-glycans comprise the structure NANA₂Gal₂GlcNAc₂Man₃GlcNAc₂, wherein such structure is afucosylated. Such structures can be produced, e.g., in engineered Pichia pastoris host cells.

In particular embodiments, the N-glycan is fucosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or α1,6-linkage fucose to produce a glycoform selected from the group consisting of Man₅GlcNAc₂(Fuc), GlcNAcMan₅GlcNAc₂(Fuc), Man₃GlcNAc₂(Fuc), GlcNAcMan₃GlcNAc₂(Fuc), GlcNAc₂Man₃GlcNAc₂(Fuc), GalGlcNAc₂Man₃GlcNAc₂(Fuc), Gal₂GlcNAc₂Man₃GlcNAc₂(Fuc), NANAGal₂GlcNAc₂Man₃GlcNAc₂(Fuc), and NANA₂Gal₂GlcNAc₂Man₃GlcNAc₂(Fuc); in an α1,3-linkage or α1,4-linkage fucose to produce a glycoform selected from the group consisting of GlcNAc(Fuc)Man₅GlcNAc₂, GlcNAc(Fuc)Man₃GlcNAc₂, GlcNAc₂(Fuc₁₋2)Man₃GlcNAc₂, GalGlcNAc₂(Fuc₁₋₂)Man₃GlcNAc₂, Gal₂GlcNAc₂(Fuc1-2)Man₃GlcNAc₂, NANAGal₂GlcNAc₂(Fuc₁₋₂)Man₃GlcNAc₂, and NANA₂Gal₂GlcNAc₂(Fuc₁₋₂)Man₃GlcNAc₂; or in an α1,2-linkage fucose to produce a glycoform selected from the group consisting of Gal(Fuc)GlcNAc₂Man₃GlcNAc₂, Gal₂(Fuc₁₋₂)GlcNAc₂Man₃GlcNAc₂, NANAGal₂(Fuc₁₋₂)GlcNAc₂Man₃GlcNAc₂, and NANA₂Gal₂(Fuc₁₋₂)GlcNAc₂Man₃GlcNAc₂.

In further aspects, the antibodies (e.g., humanized antibodies) or antigen-binding fragments thereof comprise high mannose N-glycans, including but not limited to, Man₅GlcNAc₂, Man₇GlcNAc₂, Man₆GlcNAc₂, Man₅GlcNAc₂, Man₄GlcNAc₂, or N-glycans that consist of the Man₃GlcNAc₂ N-glycan structure.

In further aspects of the above, the complex N-glycans further include fucosylated and non-fucosylated bisected and multiantennary species.

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

With respect to complex N-glycans, the terms “G−2”, “G−1”, “G0”, “G1”, “G2”, “A1”, and “A2” mean the following. “G−2” refers to an N-glycan structure that can be characterized as Man₃GlcNAc₂; the term “G−1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as NANAGal₂GlcNAc₂Man₃GlcNAc₂; and, the term “A2” refers to an N-glycan structure that can be characterized as NANA₂Gal₂GlcNAc₂Man₃GlcNAc₂. Unless otherwise indicated, the terms G−2″, “G−1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄₎Man₃GlcNAc₂, or NANA₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

Antibody Physical Properties

The antibodies and antigen-binding fragments thereof disclosed herein (e.g., hCD27.131A) may further contain one or more glycosylation sites in either the light or heavy chain immunoglobulin variable region. Such glycosylation sites may result in increased immunogenicity of the antibody or fragment thereof or an alteration of the pK of the antibody due to altered antigen-binding (Marshall et al. (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence.

Each antibody or antigen-binding fragment thereof (e.g., hCD27.131A) will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8.

Each antibody or antigen-binding fragment thereof (e.g., hCD27.131A or other humanized versions thereof) will have a characteristic melting temperature, with a higher melting temperature indicating greater overall stability in vivo (Krishnamurthy R and Manning M C 2002 Curr Pharm Biotechnol 3:361-71). In general, the T_{M1} (the temperature of initial unfolding) may be greater than 60° C., greater than 65° C., or greater than 70° C. The melting point of an antibody or fragment thereof can be measured using differential scanning calorimetry (Chen et al., 2003 Pharm Res 20:1952-60; Ghirlando et al., 1999 Immunol Lett 68:47-52) or circular dichroism (Murray et al., 2002 J. Chromatogr Sci 40:343-9).

In a further embodiment, antibodies and antigen-binding fragments thereof (e.g., antibody hCD27.131A and other humanized versions thereof) are selected that do not degrade rapidly. Degradation of an antibody or fragment thereof can be measured using capillary electrophoresis (CE) and MALDI-MS (Alexander A J and Hughes D E (1995) Anal Chem 67:3626-32).

In a further embodiment, antibodies (e.g., antibody hCD27.131A and other humanized versions thereof) and antigen-binding fragments thereof are selected that have minimal aggregation effects, which can lead to the triggering of an unwanted immune response and/or altered or unfavorable pharmacokinetic properties. Generally, antibodies and fragments are acceptable with aggregation of 25% or less, 20% or less, 15% or less, 10% or less, or 5% or less. Aggregation can be measured by several techniques, including size-exclusion column (SEC), high performance liquid chromatography (HPLC), and light scattering.

Antibody Conjugates

The anti-CD27 antibodies and antigen-binding fragments thereof disclosed herein (e.g., antibody hCD27.131A or other antigen binding fragments thereof) may also be conjugated to a chemical moiety. The chemical moiety may be, inter alia, a polymer, a radionucleotide or a cytotoxic factor. In particular embodiments, the chemical moiety is a polymer which increases the half-life of the antibody or fragment thereof in the body of a subject. Suitable polymers include, but are not limited to, hydrophilic polymers which include but are not limited to polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antigen-binding fragments thereof disclosed herein (e.g., antibody hCD27.131A and humanized versions thereof) may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷Cu, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr, and ⁵⁶Fe.

The antibodies and antigen-binding fragments disclosed herein (e.g., antibody hCD27.131A or antigen-binding fragments thereof and humanized versions thereof) may also be PEGylated, for example to increase its biological (e.g., serum) half-life. To PEGylate an antibody or fragment thereof, the antibody or fragment thereof, typically is reacted with a reactive form of polyethylene glycol (PEG), such as a reactive ester or aldehyde derivative of PEG, under conditions in which one or more PEG groups become attached to the antibody or antibody fragment thereof. In particular embodiments, the PEGylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody or fragment thereof to be PEGylated is an aglycosylated antibody or fragment thereof. Methods for PEGylating proteins are known in the art and can be applied to the antibodies of the invention. See, e.g., EP 0 154 316 and EP 0 401 384.

The antibodies and antigen-binding fragments disclosed herein (e.g., antibody hCD27.131A or antigen-binding fragments thereof) may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antigen-binding fragments thereof also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating the antibodies and antigen-binding fragments thereof of the invention (e.g., antibody hCD27.131A) to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies and fragments are conventional and very well known in the art.

Therapeutic Uses of Anti-CD27 Antibodies

Further provided are methods for treating subjects, including human subjects, in need of treatment with the isolated antibodies or antigen-binding fragments thereof disclosed herein (e.g., antibody hCD27.131A or antigen-binding fragments thereof). In one embodiment of the invention, such subject suffers from cancer, and may have some other disorder.

In various embodiments of the invention, the anti-CD27 antibody or antigen binding fragment thereof is used to treat a patient/having a solid tumor and/or a cancer characterized by presence of a solid tumor. A solid tumor in various embodiments is an abnormal mass of tissue. Solid tumors may be benign, or malignant. Different types of solid tumors are named for the type of cells that form them. Examples of solid tumors are sarcomas, carcinomas, and lymphomas. Leukemias (cancers of the blood) generally do not form solid tumors. In various embodiments the solid tumor does not contain a cyst and/or a liquid area. Exemplary solid tumors include but are not limited to sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioblastoma multiforme, astrocytoma, medulloblastoma, craniopharyngioma, ependymoma, pinealoma, hemangioblastoma, acoustic neuroma, oligodendroglioma, melanoma, neuroblastoma, and retinoblastoma.

In one embodiment, cancers that may be treated by the antibodies or antigen-binding fragments thereof disclosed herein, compositions and methods of the invention include, but are not limited to: lung cancer, pancreatic cancer, colon cancer, colorectal cancer, myeloid leukemia, acute myelogenous leukemia, chronic myelogenous leukemia, chronic myelomonocytic leukemia, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin (e.g., sarcomas), neuroblastomas, kidney carcinomas, hepatomas, non-Hodgkin's lymphoma, multiple myeloma, and anaplastic thyroid carcinoma.

A “subject” may be a mammal such as a human, dog, cat, horse, cow, mouse, rat, monkey (e.g., cynomolgus monkey, e.g., Macaca fascicularis) or rabbit. In preferred embodiments of the invention, the subject is a human subject.

“Patient” refers to any single subject for which therapy for a disease (e.g., cancer) or condition is desired or that is participating in a clinical trial, epidemiological study or used as a control, including humans and mammalian veterinary patients such as cattle, horses, dogs, and cats. In Example 1, the patient is a human patient.

The term “in association with” indicates that the components administered in a method of the present invention, e.g., an anti-CD27 antibody (e.g., humanized antibody hCD27.131A) or antigen-binding fragment thereof (e.g., antigen binding fragments of antibody hCD27.131A or another humanized version thereof) along with an anti-cancer agent can be formulated into a single composition for simultaneous delivery or formulated separately into two or more compositions (e.g., a kit). Each component can be administered to a subject at a different time than when the other component is administered; for example, each administration may be given non-simultaneously (e.g., separately or sequentially) at several intervals over a given period of time. Moreover, the separate components may be administered to a subject by the same or by a different route.

In particular embodiments, the antibodies or antigen-binding fragments thereof disclosed herein may be used alone, or in association with other, further therapeutic agents and/or therapeutic procedures, for treating or preventing any disease such as cancer, e.g., as discussed herein, in a subject in need of such treatment or prevention. Compositions, e.g., pharmaceutical compositions comprising a pharmaceutically acceptable carrier, comprising such antibodies and fragments thereof in association with further therapeutic agents are also part of the present invention.

Therefore, the present invention provides a method or use of treating cancer in a subject (e.g., a human subject/patient), comprising administering to the subject an effective amount of the antibody or antigen binding fragment thereof disclosed herein, optionally in association with a further therapeutic agent or therapeutic procedure. The present invention also provides a method of treating cancer in a human subject, comprising administering to the subject an effective amount of the antibody or antigen binding fragment thereof disclosed herein, optionally in association with a further therapeutic agent or therapeutic procedure. The present invention also provides a method of increasing the activity of an immune cell, comprising administering to a subject in need thereof an effective amount of an antibody or antigen binding fragment thereof disclosed herein. In another embodiment, the present invention provides an antibody or antigen binding fragment thereof of the invention, for use in treatment of cancer as a monotherapy; increasing the activity of an immune cell; or treatment of cancer in combination with a further therapeutic agent. In a further embodiment, the present invention provides use of the antibody or antigen binding fragment thereof of the invention for the manufacture of a medicament for increasing immune cell activation; treating cancer as a monotherapy; or treating cancer in combination with a further therapeutic agent. In another embodiment, the present invention provides a combination of an antibody or antigen binding fragment thereof of the invention and a further therapeutic agent for the treatment of cancer; increasing the activity of an immune cell; or treatment of another disorder.

In another embodiment of the invention, the subject being treated with the ant-CD27 antibody or antigen binding fragment suffers from cancer. In one embodiment the cancer is a solid tumor. In various embodiments, the cancer is osteosarcoma, rhabdomyosarcoma, neuroblastoma, kidney cancer, leukemia, renal transitional cell cancer, bladder cancer, Wilm's cancer/tumor, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, carcinoid cancer or liver cancer, breast cancer or gastric cancer. In an embodiment of the invention, the cancer is metastatic cancer, e.g., of the varieties described above.

Cancers that may be treated by the antibodies or antigen-binding fragments, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma,

leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre tumor cervical dysplasia), ovaries (ovarian carcinoma [serous cystadenocarcinoma, mucinous cystadenocarcinoma, unclassified carcinoma], granulosa thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

In other embodiments, the invention provides a method of treating cancer or treating an infection or infectious disease in a human subject, comprising administering to the subject an effective amount of an antibody or antigen binding fragment thereof of the invention, or an expression vector or a host cell according to the invention optionally in association with a further therapeutic agent or therapeutic procedure.

In particular embodiments, the antibodies or antigen-binding fragments thereof disclosed herein (e.g., antibody hCD27.131A and humanized versions thereof) may be used alone, or in association with tumor vaccines. Examples of tumor vaccines include but are not limited to vaccines for Human Papillomavirus (HPV) infection caused cancer such as Gardasil®, Gardasil® and Cervarix®; vaccines that prevent hepatitis B virus caused liver cancer such as Engerix-B® and Recombivax HB®; oncolytic virus therapy that triggers immune response such as Imlygic®; DNA vaccines such as Synchotrope MA2M plasmid DNA vaccine and ZYC101; mammaglobin-a DNA vaccine (see Clinical Cancer Res. 2014 20(23):5964-75); vector based vaccines such as PSA-TRICOM (prostvac), PANVAC-VF, Listeria monocytogenes-based PSA vaccine (see Therapeutic Advances in Vaccines, 2014, 2(5) 137-148), Listeria-mesothelin Adeno-CEA; allogeneic vaccines such as GVAX, BLP-25 (anti-Ankara-mucin 1), Belagenpumatucel-L, TG4010, CIMAvax epidermal growth factor vaccine, NY-ESO, GM.CD40L-CCL21; autologous vaccines such as: Adeno-CD40L, BCG, INGN-225, Dendritic cell vaccines such as Provenge® (Sipuleucel-T), rF-CEA-MUC1-TRICOM (panvac-DC); antigen vaccines such as MUC-1 (stimuvax), NY-ESO-1, GP-100, MAGE-A3 (melanoma antigen encoding gene A3), INGN-225 (see Pharmacology & Therapeutics 153 (2015) 1-9).

In various embodiments, the antibody or antigen binding fragment thereof is administered at one dose. For example, the subject or the patient is administered a dose of about 2 mg, about 7 mg, about 20 mg, about 30 mg, about 70 mg, about 200 mg, or about 700 mg. In various embodiments, the antibody or antigen binding fragment thereof is administered at multiple doses.

In various embodiments, the antibody or antigen binding fragment thereof is administered a dose of about 2 mg to about 700 mg. For example, the subject or patient is administered about 2 mg, about 7 mg, about 20 mg, about 30 mg, about 70 mg, about 200 mg, or about 700 mg. In various embodiments of the method, the subject or the patient is administered 2 mg of the anti-CD27 antibody or antigen binding fragment thereof. In various embodiments of the method, the subject or the patient is administered 7 mg of the anti-CD27 antibody or antigen binding fragment thereof. In various embodiments of the method, the subject or the patient is administered 20 mg of the anti-CD27 antibody or antigen binding fragment thereof. In various embodiments of the method, the subject or the patient is administered 30 mg of the anti-CD27 antibody or antigen binding fragment thereof. In various embodiments of the method, the subject or the patient is administered 70 mg of the anti-CD27 antibody or antigen binding fragment thereof. In various embodiments of the method, the subject or the patient is administered 200 mg of the anti-CD27 antibody or antigen binding fragment thereof. In various embodiments of the method, the subject or the patient is administered 700 mg of the anti-CD27 antibody or antigen binding fragment thereof. In various embodiments of the method, the subject or the patient is administered a dose of about 200 mg to about 700 mg of the anti-CD27 antibody or antigen binding fragment thereof. In various embodiments of the method, the subject or the patient is administered a dose of about 30 mg to about 700 mg of the anti-CD27 antibody or antigen binding fragment thereof. In various embodiments of the method, the subject or the patient is administered a dose of about 30 mg, about 200 mg, or about 200 mg to about 700 mg of the anti-CD27 antibody or antigen binding fragment. In various embodiments of the method, the subject or the patient is administered a dose of about 30 mg to about 200 mg of the anti-CD27 antibody or antigen binding fragment thereof. In various embodiments of the method, the subject or the patient is administered a dose of about 70 mg to about 200 mg of the anti-CD27 antibody or antigen binding fragment thereof.

In particular embodiments, the antibodies or antigen-binding fragments thereof disclosed herein may be used alone, or in association with chemotherapeutic agents.

In particular embodiments, the antibodies or antigen-binding fragments thereof disclosed herein may be used alone, or in association with radiation therapy.

In particular embodiments, the antibodies or antigen-binding fragments thereof disclosed herein (e.g., antibody hCD27.131A or antigen-binding fragments thereof and humanized versions thereof) may be used alone, or in association with targeted therapies. Examples of targeted therapies include: hormone therapies, signal transduction inhibitors (e.g., EGFR inhibitors, such as cetuximab (Erbitux) and erlotinib (Tarceva)); HER2 inhibitors (e.g., trastuzumab (Herceptin) and pertuzumab (Perj eta)); BCR-ABL inhibitors (such as imatinib (Gleevec) and dasatinib (Sprycel)); ALK inhibitors (such as crizotinib (Xalkori) and ceritinib (Zykadia)); BRAF inhibitors (such as vemurafenib (Zelboraf) and dabrafenib (Tafinlar)), gene expression modulators, apoptosis inducers (e.g., bortezomib (Velcade) and carfilzomib (Kyprolis)), angiogenesis inhibitors, e.g., bevacizumab (Avastin) and ramucirumab (Cyramza), monoclonal antibodies attached to toxins (e.g., brentuximab vedotin (Adcetris) and ado-trastuzumab emtansine (Kadcyla)).

In particular embodiments, the anti-CD27 antibodies or antigen-binding fragments thereof (e.g., antibody hCD27.131A and humanized versions thereof) may be used in combination with an anti-cancer therapeutic agent or immunomodulatory drug such as an immunomodulatory receptor inhibitor, e.g., an antibody or antigen-binding fragment thereof that specifically binds to the receptor.

In an embodiment of the invention, an anti-CD27 antibody or antigen-binding fragment thereof of the invention (e.g., antibody hCD27.131A and humanized versions thereof) is used in association with one or more of: anti-PD1 antibody, anti-PDL1 antibody, anti-TIGIT antibody, anti-CTLA4 antibody, anti-CS1 antibody (e.g., elotuzumab), anti-KIR2DL1/2/3 antibody (e.g., lirilumab), anti-CD137 antibody (e.g., urelumab), anti-GITR antibody (e.g., TRX518), anti-PD1 antibody (e.g., pembrolizumab, nivolumab, pidilizumab (CT-011)), anti-PD-L1 antibody (e.g., BMS-936559, Durvalumab, MSB0010718C or MPDL3280A), anti-PD-L2 antibody, anti-ILT1 antibody, anti-ILT2 antibody, anti-ILT3 antibody, anti-ILT4 antibody, anti-ILT5 antibody, anti-ILT6 antibody, anti-ILT7 antibody, anti-ILT8 antibody, anti-CD40 antibody, anti-OX40 antibody, anti-ICOS, anti-SIRPα, anti-KIR2DL1 antibody, anti-KIR2DL2/3 antibody, anti-KIR2DL4 antibody, anti-KIR2DL5A antibody, anti-KIR2DL5B antibody, anti-KIR3DL1 antibody, anti-KIR3DL2 antibody, anti-KIR3DL3 antibody, anti-NKG2A antibody, anti-NKG2C antibody, anti-NKG2E antibody, anti-4-1BB antibody (e.g., PF-05082566), anti-TSLP antibody, anti-IL-10 antibody, IL-10 or PEGylated IL-10, or any small organic molecule inhibitor of such targets.

In an embodiment of the invention, an anti-CD27 antibody or antigen-binding fragment thereof of the invention (e.g., antibody hCD27.131A or antigen-binding fragment thereof or a humanized version thereof) is used in association with an anti-PD1 antibody (e.g., pembrolizumab, nivolumab, pidilizumab (CT-011)).

In an embodiment of the invention, an anti-CD27 antibody or antigen-binding fragment thereof of the invention (e.g., antibody hCD27.131A or antigen-binding fragment thereof or a humanized version thereof) is used in association with an anti-PDL1 antibody (e.g., BMS-936559, Durvalumab, MSB0010718C or MPDL3280A).

In an embodiment of the invention, an anti-CD27 antibody or antigen-binding fragment thereof of the invention (e.g., antibody hCD27.131A or antigen-binding fragment thereof or a humanized version thereof) is used in association with an anti-CTLA4 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-CS1 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR2DL1/2/3 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-CD137 (e.g., urelumab) antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-GITR (e.g., TRX518) antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-PD-L2 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ITL1 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ITL2 antibody.

In an embodiment of the invention, an anti-CD27 antibody or antigen-binding fragment thereof of the invention (e.g., antibody hCD27.hCD27.131A or antigen-binding fragment thereof or a humanized version thereof) is used in association with an anti-ITL3 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ITL4 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ITL5 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ITL6 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ITL7 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ITL8 antibody.

In an embodiment of the invention, an anti-CD27 antibody or antigen-binding fragment thereof of the invention (e.g., antibody hCD27.hCD27.131A or antigen-binding fragment thereof or a humanized version thereof) is used in association with an anti-CD40 antibody.

In an embodiment of the invention, an anti-CD27 antibody or antigen-binding fragment thereof of the invention (e.g., antibody hCD27.hCD27.131A or antigen-binding fragment thereof or a humanized version thereof) is used in association with an anti-OX40 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR2DL1 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR2DL2/3 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR2DL4 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR2DL5A antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR2DL5B antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR3DL1 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR3DL2 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-KIR3DL3 antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-NKG2A antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-NKG2C antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-ICOS antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-SIRPα antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-4-1BB antibody.

In an embodiment of the invention, an anti-CD27 antibody or antigen-binding fragment thereof of the invention (e.g., antibody hCD27.131A or antigen-binding fragment thereof or a humanized version thereof) is used in association with an anti-IL-10 antibody.

In an embodiment of the invention, an anti-CD27 antibody or antigen-binding fragment thereof of the invention (e.g., antibody hCD27.hCD27.131A or antigen-binding fragment thereof or a humanized version thereof) is used in association with an anti-TSLP antibody.

In an embodiment of the invention, an anti-CD27 antibody or antigen-binding fragment thereof of the invention (e.g., antibody hCD27.131A or antigen-binding fragment thereof or a humanized version thereof) is used in association with IL-10 or PEGylated IL-10.

In an embodiment of the invention, an anti-CD27 antibody or antigen-binding fragment thereof of the invention (e.g., antibody hCD27.131A or antigen-binding fragment thereof or a humanized version thereof) is used in association with one or more of an inhibitor (e.g., a small organic molecule or an antibody or antigen-binding fragment thereof) such as: an MTOR (mammalian target of rapamycin) inhibitor, a cytotoxic agent, a platinum agent, an EGFR inhibitor, a VEGF inhibitor, a microtubule stabilizer, a taxane, a CD20 inhibitor, a CD52 inhibitor, a CD30 inhibitor, a RANK (Receptor activator of nuclear factor kappa-B) inhibitor, a STING agonist, a CXCR2 antagonist, a RANKL (Receptor activator of nuclear factor kappa-B ligand) inhibitor, an ERK inhibitor, a MAP Kinase inhibitor, an AKT inhibitor, a MEK inhibitor, a PARP inhibitor, a PI3K inhibitor, a HER1 inhibitor, a HER2 inhibitor, a HER3 inhibitor, a HER4 inhibitor, a Bcl2 inhibitor, a CD22 inhibitor, a CD79b inhibitor, an ErbB2 inhibitor, or a farnesyl protein transferase inhibitor.

In an embodiment of the invention, an anti-CD27 antibody or antigen-binding fragment thereof of the invention (e.g., antibody hCD27.131A or a humanized version thereof) is used in association with any one or more of: 13-cis-retinoic acid, 3-[5-(methylsulfonylpiperadinemethyl)-indolyl]-quinolone, 4-hydroxytamoxifen, 5-deooxyuridine, 5′-deoxy-5-fluorouridine, 5-fluorouracil, 6-mecaptopurine, 7-hydroxystaurosporine, A-443654, abirateroneacetate, abraxane, ABT-578, acolbifene, ADS-100380, ALT-110, altretamine, amifostine, aminoglutethimide, amrubicin, Amsacrine, anagrelide, anastrozole, angiostatin, AP-23573, ARQ-197, arzoxifene, AS-252424, AS-605240, asparaginase, AT-9263, atrasentan, axitinib, AZD1152, Bacillus Calmette-Guerin (BCG) vaccine, batabulin, BC-210, besodutox, bevacizumab, bicalutamide, Bio111, BIO140, bleomycin, BMS-214662, BMS-247550, BMS-275291, BMS-310705, bortezimib, buserelin, busulfan, calcitriol, camptothecin, canertinib, capecitabine, carboplatin, carmustine, CC8490, Cediranib, CG-1521, CG-781, chlamydocin, chlorambucil, chlorotoxin, cilengitide, cimitidine, cisplatin, cladribine, clodronate, COL-3, CP-724714, cyclophosphamide, cyproterone, cyproteroneacetate, cytarabine, cytosinearabinoside, dacarbazine, dacinostat, dactinomycin, dalotuzumab, danusertib, dasatanib, daunorubicin, decatanib, deguelin, denileukin, deoxycoformycin, depsipeptide, diarylpropionitrile, diethylstilbestrol, diftitox, docetaxel, dovitinib, doxorubicin, droloxifene, edotecarin, yttrium-90 labeled-edotreotide, edotreotide, EKB-569, EMD121974, endostatin, enzalutamide, enzastaurin, epirubicin, epithilone B, ERA-923, Erbitux, erlotinib, estradiol, estramustine, etoposide, everolimus, exemestane, ficlatuzumab, finasteride, flavopiridol, floxuridine, fludarabine, fludrocortisone, fluoxymesterone, flutamide, FOLFOX regimen, Fulvestrant, galeterone, gefitinib, gemcitabine, gimatecan, goserelin, goserelin acetate, gossypol, GSK461364, GSK690693, HMR-3339, hydroxyprogesteronecaproate, hydroxyurea, IC87114, idarubicin, idoxyfene, ifosfamide, IM862, imatinib, IMC-1C11, INCB24360, INO1001, interferon, interleukin-12, ipilimumab, irinotecan, JNJ-16241199, ketoconazole, KRX-0402, lapatinib, lasofoxifene, letrozole, leucovorin, leuprolide, leuprolide acetate, levamisole, liposome entrapped paclitaxel, lomustine, lonafarnib, lucanthone, LY292223, LY292696, LY293646, LY293684, LY294002, LY317615, marimastat, mechlorethamine, medroxyprogesteroneacetate, megestrolacetate, melphalan, mercaptopurine, mesna, methotrexate, mithramycin, mitomycin, mitotane, mitoxantrone, tozasertib, MLN8054, neovastat, Neratinib, neuradiab, nilotinib, nilutimide, nolatrexed, NVP-BEZ235, oblimersen, octreotide, ofatumumab, olaparib, oregovomab, orteronel, oxaliplatin, paclitaxel, palbociclib, pamidronate, panitumumab, pazopanib, PD0325901, PD184352, PEG-interferon, pemetrexed, pentostatin, perifosine, phenylalaninemustard, PI-103, pictilisib, PIK-75, pipendoxifene, PKI-166, plicamycin, porfimer, prednisone, procarbazine, progestins, PX-866, R-763, raloxifene, raltitrexed, razoxin, ridaforolimus, rituximab, romidepsin, RTA744, rubitecan, scriptaid, Sdx102, seliciclib, selumetinib, semaxanib, SF1126, sirolimus, SN36093, sorafenib, spironolactone, squalamine, SR13668, streptozocin, SU6668, suberoylanalide hydroxamic acid, sunitinib, synthetic estrogen, talampanel, talimogene laherparepvec, tamoxifen, temozolomide, temsirolimus, teniposide, tesmilifene, testosterone, tetrandrine, TGX-221, thalidomide, thioguanine, thiotepa, ticilimumab, tipifarnib, tivozanib, TKI-258, TLK286, topotecan, toremifene citrate, trabectedin, trastuzumab, tretinoin, trichostatin A, triciribinephosphate monohydrate, triptorelin pamoate, TSE-424, uracil mustard, valproic acid, valrubicin, vandetanib, vatalanib, VEGF trap, vinblastine, vincristine, vindesine, vinorelbine, vitaxin, vitespan, vorinostat, VX-745, wortmannin, Xr311, zanolimumab, ZK186619, ZK-304709, ZM336372, ZSTK474.

Non-limiting examples of suitable anti-cancer agents to be used in combination with an anti-CD27 antibody or antigen-binding fragment thereof of the invention include cytostatic agents, cytotoxic agents, targeted therapeutic agents (e.g., small molecules, biologics, siRNA and microRNA) against cancer and neoplastic diseases,

1) anti-metabolites (such as methoxtrexate, 5-fluorouracil, gemcitabine, fludarabine, capecitabine);

2) alkylating agents, such as temozolomide, cyclophosphamide,

3) DNA interactive and DNA damaging agents, such as cisplatin, oxaliplatin, doxorubicin,

4) Ionizing irradiation, such as radiation therapy,

5) topoisomerase II inhibitors, such as etoposide, doxorubicin,

6) topoisomerase I inhibitors, such as irinotecan, topotecan,

7) tubulin interacting agents, such as paclitaxel, docetaxel, Abraxane, epothilones,

8) kinesin spindle protein inhibitors,

9) spindle checkpoint inhibitors,

10) Poly(ADP-ribose) polymerase (PARP) inhibitors, such as olaparib, niraparib and veliparib

11) Matrix metalloprotease (MMP) inhibitors

12) Protease inhibitors, such as cathepsin D and cathepsin K inhibitors

13) Proteosome or ubiquitination inhibitors, such as bortezomib,

14) Activator of mutant p53 to restore its wild-type p53 activity

15) Adenoviral-p53

16) Bcl-2 inhibitors, such as ABT-263

17) Heat shock protein (HSP) modulators, such as geldanamycin and 17-AAG

18) Histone deacetylase (HDAC) inhibitors, such as vorinostat (SAHA),

19) sex hormone modulating agents,

a. anti-estrogens, such as tamoxifen, fulvestrant,

b. selective estrogen receptor modulators (SERM), such as raloxifene,

c. anti-androgens, such as bicalutamide, flutamide

d. LHRH agonists, such as leuprolide,

e. 5α-reductase inhibitors, such as finasteride,

f. Cytochrome P450 C17 lyase (CYP450c17, also called 17αC);

g. aromatase inhibitors, such as letrozole, anastrozole, exemestane,

20) EGFR kinase inhibitors, such as geftinib, erlotinib, laptinib

21) dual erbB1 and erbB2 inhibitors, such as Lapatinib

22) multi-targeted kinases (serine/threonine and/or tyrosine kinase) inhibitors,

a. ABL kinase inhibitors, imatinib and nilotinib, dasatinib

b. VEGFR-1, VEGFR-2, PDGFR, KDR, FLT, c-Kit, Tie2, Raf, MEK and ERK inhibitors, such as sunitinib, sorafenib, Vandetanib, pazopanib, PLX-4032, Axitinib, PTK787, GSK-1120212

c. Polo-like kinase inhibitors

d. Aurora kinase inhibitors

e. JAK inhibitor

f. c-MET kinase inhibitors

g. PI3K and mTOR inhibitors, such as GDC-0941, BEZ-235, BKM-120 and AZD-8055

h. Rapamycin and its analogs, such as Temsirolimus, everolimus, and deforolimus

i. STING (Stimulator of Interferon Genes) agonist

j. CXCR (CXC Chemokine Receptor) inhibitor, CXCR2 antagonist

23) and other anti-cancer (also known as anti-neoplastic) agents include but are not limited to ara-C, adriamycin, cytoxan, Carboplatin, Uracil mustard, Clormethine, Ifosfsmide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, Pentostatine, Vinblastine, Vincristine, Vindesine, Vinorelbine, Navelbine, Bleomycin, Dactinomycin, Daunorubicin, Doxorubicin, Epirubicin, teniposide, cytarabine, pemetrexed, Idarubicin, Mithramycin, Deoxycoformycin, Mitomycin-C, L-Asparaginase, Teniposide, Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Flutamide Medroxyprogesteroneacetate, Toremifene, goserelin, Carboplatin, Hydroxyurea, Amsacrine, Procarbazine, Mitotane, Mitoxantrone, Levamisole, Drolloxafine, Hexamethylmelamine, Bexxar, Zevalin, Trisenox, Profimer, Thiotepa, Altretamine, Doxil, Ontak, Depocyt, Aranesp, Neupogen, Neulasta, Kepivance.

24) Farnesyl protein transferase inhibitors, such as, SARASAR™ (4-[2-[4-[(11R)-3,10-dibromo-8-chloro-6,11-dihydro-5H-benzo[5,6]cyclohepta[1,2-b]pyridin-11-yl-]-1-
25 piperidinyl]-2-oxoethyl]-piperidinecarboxamide, tipifarnib
25) interferons, such as Intron A, Peg-Intron,
26) anti-erbB1 antibodies, such as cetuximab, panitumumab,
27) anti-erbB2 antibodies, such as trastuzumab,
28) anti-CD52 antibodies, such as Alemtuzumab,
29) anti-CD20 antibodies, such as Rituximab
30) anti-CD33 antibodies, such as Gemtuzumab ozogamicin
31) anti-VEGF antibodies, such as Avastin,
32) TRIAL ligands, such as Lexatumumab, mapatumumab, and AMG-655
33) anti-CTLA-4 antibodies, such as ipilimumab
34) antibodies against CTA1, CEA, CD5, CD19, CD22, CD30, CD44, CD44V6, CD55, CD56, EpCAM, FAP, MHCII, HGF, IL-6, MUC1, PSMA, TALE, TAG-72, TRAILR, VEGFR, IGF-2, FGF,

35) anti-IGF-1R antibodies, such as dalotuzumab and robatumumab (SCH 717454).

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, platinum coordinator compounds, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, 4-demethoxy-3-deamino-3-aziridinyl-4-methylsulphonyl-daunorubicin (see WO 00/50032).

An example of a hypoxia activatable compound is tirapazamine.

Examples of proteosome inhibitors include but are not limited to lactacystin and MLN-341 (Velcade).

Examples of microtubule inhibitors/microtubule-stabilising agents include taxanes in general. Specific compounds include paclitaxel (Taxol®), vindesine sulfate, 3′,4′-didehydro-4′-deoxy-8′-norvincaleukoblastine, docetaxol (Taxotere®), rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c] quinolin-7-one, and dimesna.

Examples of inhibitors of mitotic kinesins, and in particular the human mitotic kinesin KSP, are described in patent publication numbers WO03/039460, WO03/050064, WO03/050122, WO03/049527, WO03/049679, WO03/049678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604 181, European Patent Publ. 0 696 593, WO94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO96/34851, WO 96/30017, WO 96/30018, WO 96/30362, WO 96/30363, WO 96/31111, WO96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76(1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO2004/096135, WO 2004/096130, WO 2005/100356, WO 2005/100344, US 2005/029941, US 2005/44294, US 2005/43361, 60/734,188, 60/652,737, 60/670,469), inhibitors of Raf kinase (for example PLX-4032), inhibitors of MEK (for example Arry-162, RO-4987655 and GSK-1120212), inhibitors of mTOR (for example AZD-8055, BEZ-235 and everolimus), and inhibitors of PI3K (for example GDC-0941, BKM-120).

As used above, “integrin blockers” refers to compounds which selectively antagonize, inhibit or counteract binding of a physiological ligand to the α_{V}β₃ integrin, to compounds which selectively antagonize, inhibit or counteract binding of a physiological ligand to the αvβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, αᵥβ₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy) quinazolinamine, BIBX1382, 2,3,9,10,11,12-hexahydro-10-(hydroxymethyl)-10-hydroxy methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations of the anti-CD27 antibodies or antigen binding fragments with PPAR-γ (i.e., PPAR-gamma) agonists and PPAR-δ (i.e., PPAR-delta) agonists may be useful in the treatment of certain malignancies. PPAR-γ and PPAR-δ are the nuclear peroxisome proliferator-activated receptors γ and δ. The expression of PPAR-γ on endothelial cells and its involvement in angiogenesis has been reported in the literature (see Agarwal J. Cardiovasc. Pharmacol. 1998; 31:909-913; J. Biol. Chem. 1999; 274:9116-9121; Murata et al., Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, Lynparza®, Rucaparib®, Talazoparib®, niraparib, Veliparib®, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926,2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy) phenoxy)propoxy)-2-ethylchromane-2-carboxylic acid.

The antibody or antigen binding fragment thereof of the instant invention may also be useful for treating or preventing breast cancer in combination with aromatase inhibitors. Examples of aromatase inhibitors include but are not limited to: anastrozole, letrozole and exemestane.

The antibody or antigen binding fragment thereof of the instant invention may also be useful for treating cancer in combination with the following chemotherapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bendamustine hydrochloride (Treanda®); bevacuzimab (Avastin®); bexarotene capsules (Targretin®); bexarotene gel (Targretin®); bleomycin (Blenoxane®); bortezomib (Velcade®); brefeldin A; busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); dalteparin sodium injection (Fragmin®); Darbepoetin alfa (Aranesp®); dasatinib (Sprycel®); daunorubicin liposomal (DanuoXome®); daunorubicin, daunomycin (Daunorubicin®); daunorubicin, daunomycin (Cerubidine®); degarelix (Firmagon®); Denileukin diftitox (Ontak®); dexrazoxane (Zinecard®); dexrazoxane hydrochloride (Totect®); didemnin B; 17-DMAG; docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); eculizumab injection (Soliris®); Elliott's B Solution (Elliott's B Solution®); eltrombopag (Promacta®); epirubicin (Ellence®); Epoetin alfa (Epogen®); erlotinib (Tarceva®); estramustine (Emcyt®); ethinyl estradiol; etoposide phosphate (Etopophos®); etoposide, VP-16 (Vepesid®); everolimus tablets (Afinitor®); exemestane (Aromasin®); ferumoxytol (Feraheme Injection®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); geldanamycin; gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); iobenguane I 123 injection (AdreView®); irinotecan (Camptosar®); ixabepilone (Ixempra®); lapatinib tablets (Tykerb®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); 8-methoxypsoralen; mitomycin C (Mutamycin®); mitotane (Lysodren®); mitoxantrone (Novantrone®); mitramycin; nandrolone phenpropionate (Durabolin-50®); nelarabine (Arranon®); nilotinib (Tasigna®); Nofetumomab (Verluma®); ofatumumab (Arzerra®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); panitumumab (Vectibix®); pazopanib tablets (Votrienttm®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plerixafor (Mozobil®); plicamycin, mithramycin (Mithracin®); porfimer sodium (Photofrin®); pralatrexate injection (Folotyn®); procarbazine (Matulane®); quinacrine (Atabrine®); rapamycin; Rasburicase (Elitek®); raloxifene hydrochloride (Evista®); Rituximab (Rituxan®); romidepsin (Istodax®); romiplostim (Nplate®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); temsirolimus (Torisel®); teniposide, VM-26 (Vumon®); testolactone (Teslac®); thioguanine, 6-TG (Thioguanine®); thiopurine; thiotepa (Thioplex®); topotecan (Hycamtin®); toremifene (Fareston®); Tositumomab (Bexxar®); Tositumomab/I-131 tositumomab (Bexxar®); trans-retinoic acid; Trastuzumab (Herceptin®); tretinoin, ATRA (Vesanoid®); triethylenemelamine; Uracil Mustard (Uracil Mustard Capsules®); valrubicin (Valstar®); vinblastine (Velban®); vincristine (Oncovin®); vinorelbine (Navelbine®); vorinostat (Zolinza®); wortmannin; and zoledronate (Zometa®).

In an embodiment of the invention, an anti-CD27 antibody or antigen-binding fragment thereof of the invention (e.g., antibody hCD27.131A or a humanized version thereof) is used in association with one or more antiemetics including, but not limited to: casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Other side effects of cancer treatment include red and white blood cell deficiency.

Accordingly, in an embodiment of the invention, an anti-CD27 antibody or antigen-binding fragment thereof (e.g., antibody hCD27.131A or antigen-binding fragment thereof or a humanized version thereof) is in association with an agent which treats or prevents such a deficiency, such as, e.g., filgrastim, PEG-filgrastim, erythropoietin, epoetin alfa or darbepoetin alfa.

In an embodiment of the invention, an anti-CD27 antibody or antigen-binding fragment thereof of the invention (e.g., antibody hCD27.131A or antigen-binding fragment thereof or a humanized version thereof) is administered in association with anti-cancer radiation therapy. For example, in an embodiment of the invention, the radiation therapy is external beam therapy (EBT): a method for delivering a beam of high-energy X-rays to the location of the tumor. The beam is generated outside the patient (e.g., by a linear accelerator) and is targeted at the tumor site. These X-rays can destroy the cancer cells and careful treatment planning allows the surrounding normal tissues to be spared. No radioactive sources are placed inside the patient's body. In an embodiment of the invention, the radiation therapy is proton beam therapy: a type of conformal therapy that bombards the diseased tissue with protons instead of X-rays. In an embodiment of the invention, the radiation therapy is conformal external beam radiation therapy: a procedure that uses advanced technology to tailor the radiation therapy to an individual's body structures. In an embodiment of the invention, the radiation therapy is brachytherapy: the temporary placement of radioactive materials within the body, usually employed to give an extra dose—or boost—of radiation to an area.

In an embodiment of the invention, a surgical procedure is administered in association with an anti-CD27 antibody or antigen-binding fragment thereof (e.g., antibody hCD27.131A or antigen-binding fragment thereof or a humanized version thereof) is surgical tumorectomy.

In a further embodiment, the patient is infused with autologous T cells expanded ex vivo with anti-CD27 specific antibodies or antigen-binding fragments thereof. In another embodiment, the patient is administered autologous T cells in combination with the anti-CD27 specific antibodies or antigen-binding fragments thereof. In yet another embodiment, the patient is vaccinated with a cancer vaccine, and infused with autologous T cells expanded ex vivo with anti-CD27 specific antibodies or antigen-binding fragments thereof. The autologous T-cells can be autologous infiltrating lymphocytes, T-cells transduced with high affinity T-cell receptors against tumor antigens or T cells transduced with chimeric antigen receptors composed of hybrid immunoglobulin lights chains with endo-domains of T-cell signaling molecules. See Kalos M. and June C. H., Immunity, 39, 2013, p 49-60; Wu R. et al, Cancer J. 2012; 18(2): 160-175; and June, C. H., J. Clin. Invest. 117:1466-1476 (2007).

Pharmaceutical Compositions and Administration

To prepare pharmaceutical or sterile compositions of the anti-CD27 antibodies and antigen-binding fragments of the invention (e.g., antibody hCD27.131A or antigen-binding fragment thereof and humanized versions thereof), the antibody or antigen-binding fragment thereof is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Toxicity and therapeutic efficacy of the antibodies or antigen binding fragments thereof, administered alone or in combination with another therapeutic agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In a further embodiment, a further therapeutic agent that is administered to a subject in association with an anti-CD27 antibody or antigen-binding fragment thereof of the invention in accordance with the Physicians' Desk Reference 2003 (Thomson Healthcare; 57th edition (Nov. 1, 2002)).

In various embodiments, the anti-CD27 antibody or antigen binding fragment thereof is administered at about 2 mg to about 700 mg. For example, the anti-CD27 antibody or antigen binding fragment thereof is administered at about 2 mg, about 7 mg, 20 mg, 30 mg, 70 mg, 200 mg, or about 700 mg.

The mode of administration can vary. Routes of administration (e.g., anti-CD27 antibody or antigen binding fragment thereof and the PD-1 antibody or the antigen binding fragment thereof) include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. For example, the anti-CD27 antibody or antigen binding fragment thereof is administered intravenously or subcutaneously.

In particular embodiments, the anti-CD27 antibodies or antigen-binding fragments thereof of the invention can be administered by an invasive route such as by injection. In further embodiments of the invention, an anti-CD27 antibody or antigen-binding fragment thereof, or pharmaceutical composition thereof, is administered intravenously, subcutaneously, intramuscularly, intraarterially, intratumorally, or by inhalation, aerosol delivery. Administration by non-invasive routes (e.g., orally; for example, in a pill, capsule or tablet) is also within the scope of the present invention.

A vessel (e.g., a plastic or glass vial, e.g., with a cap or a chromatography column, hollow bore needle or a syringe cylinder) may be used that contains any of the antibodies or antigen-binding fragments thereof (e.g., antibody hCD27.131A and humanized versions thereof) or a pharmaceutical composition thereof. The present invention also provides an injection device comprising any of the antibodies or antigen-binding fragments thereof of the invention or a pharmaceutical composition thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., antibody or fragment thereof or a pharmaceutical composition thereof), a needle for piecing skin and/or blood vessels for injection of the fluid; and a plunger for pushing the fluid out of the cylinder and through the needle bore. In an embodiment of the invention, an injection device that comprises an antibody or antigen-binding fragment thereof of the present invention or a pharmaceutical composition thereof is an intravenous (IV) injection device. Such a device includes the antibody or fragment thereof or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂) and optionally including glucose) introduced into the body of the patient through the cannula or trocar/needle. The antibody or fragment thereof or a pharmaceutical composition thereof may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the antibody or fragment thereof or a pharmaceutical composition thereof to a patient's body. External infusion pumps are medical devices that deliver the antibody or fragment thereof or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556. Such needleless devices comprising the pharmaceutical composition are also part of the present invention. The pharmaceutical compositions disclosed herein may also be administered by infusion. Examples of well-known implants and modules for administering the pharmaceutical compositions include those disclosed in: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art and those comprising the pharmaceutical compositions of the present invention are within the scope of the present invention.

Alternately, one may administer the anti-CD27 antibody or antigen-binding fragment thereof of the invention in a local rather than systemic manner, for example, via injection of the antibody or fragment thereof directly into a tumor, e.g., a CD27⁺ tumor. Furthermore, one may administer the antibody or fragment thereof in a targeted drug delivery system, for example, in a liposome coated with a tissue-specific antibody, targeting, for example, a tumor e.g., a CD27⁺ tumor, e.g., characterized by immunopathology. The liposomes will be targeted to and taken up selectively by the afflicted tissue. Such methods and liposomes are part of the present invention.

The administration regimen depends on several factors, including the serum or tissue turnover rate of the therapeutic antibody or antigen-binding fragment thereof, the level of symptoms, the immunogenicity of the therapeutic antibody, and the accessibility of the target cells in the biological matrix. Preferably, the administration regimen delivers sufficient therapeutic antibody or fragment thereof to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies or fragments is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602). In various embodiments, the patient is provided a short repeated exposure of the antibody or antigen binding fragment thereof rather than continuous exposure. Alternatively, the patient is provided a continuous exposure.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, humanized and fully human antibodies may be desirable.

Antibodies or antigen-binding fragments thereof disclosed herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. (see, e.g., Yang, et al., 2003 New Engl. J. Med. 349:427-434; Herold, et al., 2002 New Engl. J. Med. 346:1692-1698; Liu, et al., 1999 J. Neurol. Neurosurg. Psych. 67:451-456; Portielji, et al., 2003 Cancer Immunol. Immunother. 52:151-144). Doses may also be provided to achieve a pre-determined target concentration of anti-CD27 antibody in the subject's serum.

Kits

Further provided are kits comprising one or more components that include, but are not limited to, an anti-CD27 antibody or antigen-binding fragment thereof, as discussed herein (e.g., as described in Table 1) alone or in association with one or more additional components including, but not limited to a pharmaceutically acceptable carrier and/or a therapeutic agent, as discussed herein. The antibody or fragment thereof and/or the therapeutic agent can be formulated as a pure composition or in combination with a pharmaceutically acceptable carrier, in a pharmaceutical composition.

In one embodiment, the kit includes an anti-CD27 antibody or antigen-binding fragment thereof of the invention or a pharmaceutical composition thereof in one container (e.g., in a sterile glass or plastic vial) and/or a therapeutic agent and a pharmaceutical composition thereof in another container (e.g., in a sterile glass or plastic vial).

In another embodiment, the kit comprises a combination of the invention, including an anti-CD27 antibody or antigen-binding fragment thereof of the invention along with a pharmaceutically acceptable carrier, optionally in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

Therapeutic Kits

As a matter of convenience, an anti-CD27 antibody or antigen-binding fragment thereof of the invention can be provided in a kit, i.e., a packaged combination of reagents in predetermined amounts with instructions for performing the therapeutic assay.

Also provided is a kit comprising an anti-CD27 antibody (e.g., humanized antibody) or antigen-binding fragment thereof packaged in a container, such as a vial or bottle, and further comprising a label attached to or packaged with the container, the label describing the contents of the container and providing indications and/or instructions regarding use of the contents of the container to treat one or more disease states as described herein.

In one aspect, the kit is for treating cancer and comprises an anti-CD27 antibody (e.g., a humanized antibody for example listed in Table 1) or antigen-binding fragment thereof and a further therapeutic agent or a vaccine. The kit may optionally further include a syringe for parenteral, e.g., intravenous, administration. In another aspect, the kit comprises an anti-CD27 antibody (e.g., humanized antibody for example listed in Table 1) or antigen-binding fragment thereof and a label attached to or packaged with the container describing use of the antibody or fragment with the vaccine or further therapeutic agent. In yet another aspect, the kit comprises the vaccine or further therapeutic agent and a label attached to or packaged with the container describing use of the vaccine or further therapeutic agent with the anti-CD27 antibody or fragment. In certain embodiments, an anti-CD27 antibody and vaccine or further therapeutic agent are in separate vials or are combined together in the same pharmaceutical composition. In addition to the tumor vaccines described above, vaccines for infectious disease may be used in combination with the anti-CD27 antibody or antigen-binding fragment thereof, for example, COMVAX®, M-M-R® II, Pedvax HIB®, PNEUMOVAX® 23, ProQuad®, RotaTeq®, VARIVAX®, and ZOSTAVAX®.

As discussed above in the combination therapy section, concurrent administration of two therapeutic agents may not require that the agents be administered at the same time or by the same route, as long as there is an overlap in the time period during which the agents are exerting their therapeutic effect. Simultaneous or sequential administration is contemplated, as is administration on different days or weeks.

The therapeutic disclosed herein may also be prepared that comprise at least one of the antibody, peptide, antigen-binding fragment thereof, or polynucleotide disclosed herein and instructions for using the composition as a detection reagent or therapeutic agent. Containers for use in such kits may typically comprise at least one vial, test tube, flask, bottle, syringe or other suitable container, into which one or more of the detection and/or therapeutic composition(s) may be placed, and preferably suitably aliquoted. Where a second therapeutic agent is also provided, the kit may also contain a second distinct container into which this second detection and/or therapeutic composition may be placed. Alternatively, a plurality of compounds may be prepared in a single pharmaceutical composition, and may be packaged in a single container means, such as a vial, flask, syringe, bottle, or other suitable single container. The kits disclosed herein will also typically include a means for containing the vial(s) in close confinement for commercial sale, such as, e.g., injection or blow-molded plastic containers into which the desired vial(s) are retained. Where a radiolabel, chromogenic, fluorigenic, or other type of detectable label or detecting means is included within the kit, the labeling agent may be provided either in the same container as the detection or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and/or convenient packaging and delivery.

A device or apparatus for carrying out the detection or monitoring methods described herein is also provided. Such an apparatus may include a chamber or tube into which sample can be input, a fluid handling system optionally including valves or pumps to direct flow of the sample through the device, optionally filters to separate plasma or serum from blood, mixing chambers for the addition of capture agents or detection reagents, and optionally a detection device for detecting the amount of detectable label bound to the capture agent immunocomplex. The flow of sample may be passive (e.g., by capillary, hydrostatic, or other forces that do not require further manipulation of the device once sample is applied) or active (e.g., by application of force generated via mechanical pumps, electroosmotic pumps, centrifugal force, or increased air pressure), or by a combination of active and passive forces.

In further embodiments, also provided is a processor, a computer readable memory, and a routine stored on the computer readable memory and adapted to be executed on the processor to perform any of the methods described herein. Examples of suitable computing systems, environments, and/or configurations include personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, or any other systems known in the art.

General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al., 2000 Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al., 2000 Current Protocols in Protein Science, Vol. 2, John Wiley and Sons, Inc., New York; Ausubel, et al., 2001 Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al., 2001 Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al., 2001 Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al., 2000 J. Immunol. 165:6205; He, et al., 1998 J. Immunol. 160:1029; Tang et al., 1999 J. Biol. Chem. 274:27371-27378; Baca et al., 1997 J. Biol. Chem. 272:10678-10684; Chothia et al., 1989 Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al., 1996 Nature Biotechnol. 14:309-314; Barbas, 1995 Nature Medicine 1:837-839; Mendez et al., 1997 Nature Genetics 15:146-156; Hoogenboom and Chames 2000 Immunol. Today 21:371-377; Barbas et al., 2001 Phage Display: A Laboratory Manual, Cold Spring Harbor Laboratory Press, Cold Spring Harbor, N.Y.; Kay et al., 1996 Phage Display of Peptides and Proteins: A Laboratory Manual, Academic Press, San Diego, Calif.; de Bruin et al., 1999 Nature Biotechnol. 17:397-399).

Single chain antibodies and diabodies are described (see, e.g., Malecki et al., 2002 Proc. Natl. Acad. Sci. USA 99:213-218; Conrath et al., 2001 J. Biol. Chem. 276:7346-7350; Desmyter et al., 2001 J. Biol. Chem. 276:26285-26290; Hudson and Kortt, 1999 J. Immunol. Methods 231:177-189; and U.S. Pat. No. 4,946,778). Bifunctional antibodies are provided (see, e.g., Mack, et al., 1995 Proc. Natl. Acad. Sci. USA 92:7021-7025; Carter, 2001 J. Immunol. Methods 248:7-15; Volkel, et al., 2001 Protein Engineering 14:815-823; Segal, et al., 2001 J. Immunol. Methods 248:1-6; Brennan, et al., 1985 Science 229:81-83; Raso, et al., 1997 J. Biol. Chem. 272:27623; Morrison, 1985 Science 229:1202-1207; Traunecker, et al., 1991 EMBO J. 10:3655-3659; and U.S. Pat. Nos. 5,932,448, 5,532,210, and 6,129,914).

Bispecific antibodies are also provided (see, e.g., Azzoni et al., 1998 J. Immunol. 161:3493; Kita et al., 1999 J. Immunol. 162:6901; Merchant et al., 2000 J. Biol. Chem. 74:9115; Pandey et al., 2000 J. Biol. Chem. 275:38633; Zheng et al., 2001 J. Biol Chem. 276:12999; Propst et al., 2000 J. Immunol. 165:2214; Long, 1999 Ann. Rev. Immunol. 17:875).

Purification of antigen is not necessary for the generation of antibodies. Animals can be immunized with cells bearing the antigen of interest. Splenocytes can then be isolated from the immunized animals, and the splenocytes can be fused with a myeloma cell line to produce a hybridoma (see, e.g., Meyaard et al., 1997 Immunity 7:283-290; Wright et al., 2000 Immunity 13:233-242; Preston et al., supra; Kaithamana et al., 1999 J. Immunol. 163:5157-5164).

Antibodies can be conjugated, e.g., to small drug molecules, enzymes, liposomes, and polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al., 1991 J. Immunol. 146:169-175; Gibellini et al., 1998 J. Immunol. 160:3891-3898; Hsing and Bishop, 1999 J. Immunol. 162:2804-2811; Everts et al., 2002 J. Immunol. 168:883-889).

Methods for flow cytometry, including fluorescence activated cell sorting (FACS), are available (see, e.g., Owens, et al., 1994 Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al., 2000 Bioinformatics 16: 741-742; Menne, et al., 2000 Bioinformatics Applications Note 16:741-742; Wren, et al., 2002 Comput. Methods Programs Biomed. 68:177-181; von Heijne, 1983 Eur. J. Biochem. 133:17-21; von Heijne, 1986 Nucleic Acids Res. 14:4683-4690).

### Example 1: A Phase 1 Study of CD-27 Antibody hCD27.131A as a Monotherapy and in Combination with Pembrolizumab in Participants with Advanced Solid Tumors

A multi-site, multi-arm, dose escalation, dose confirmation, dose expansion phase 1 study was performed. Humanized antibody hCD27.131A is being developed for the treatment of solid tumors. This study was the first-in-human study of antibody hCD27.131A and was designed to assess the safety, tolerability, pharmacokinetics (PK), and pharmacodynamics (PD) of escalating doses of antibody hCD27.131A when used as monotherapy and in combination with pembrolizumab in participants with advanced solid tumors who have received or been intolerant to all treatment known to confer clinical benefit. The effect of antibody hCD27.131A on tumor size was explored.

In addition, the Arm 3 cohort in the dose escalation/dose confirmation phase of the study assessed the safety and tolerability of treatment with antibody hCD27.131A in combination with pembrolizumab and standard chemotherapy in participants with non-squamous NSCLC.

The dose expansion phase of the study further examined safety and exploratory efficacy of antibody hCD27.131A when used as monotherapy and in combination with pembrolizumab in specific tumor types as described herein. See Table 5.

Participants in the dose escalation/dose confirmation phase were allocated to 1 of 2 treatment arms:
Arm 1: Escalating doses of humanized antibody hCD27.131A given once every 3 weeks (Q3W) as monotherapy; or
Arm 2: Escalating doses of humanized antibody hCD27.131A in combination with 200 mg pembrolizumab, both given Q3W.

Six pre-determined dose levels of antibody hCD27.131A were explored independently in each arm: 2 mg, 7 mg, 20 mg, 70 mg, 200 mg and 700 mg. The dose of pembrolizumab in Arm 2 remained constant at 200 mg. All treatments were given by IV infusion Q3W.

Participants in the dose escalation/dose confirmation phase with non-squamous NSCLC enrolled were allocated to:
Arm 3: 30 mg humanized antibody hCD27.131A in combination with 200 mg pembrolizumab, 500 mg/m² pemetrexed, and AUC 5 mg/mL/min carboplatin, all given Q3W.

Participants in the dose expansion phase with triple-negative breast cancer (TNBC) were allocated to: Arm 2a: 30 mg humanized antibody hCD27.131A in combination with 200 mg pembrolizumab, both given Q3W.

Participants in the dose expansion phase with endometrial cancer were allocated to:
Arm 1a: 30 mg humanized antibody hCD27.131A given Q3W as monotherapy;
Arm 2b: 30 mg humanized antibody hCD27.131A in combination with 200 mg pembrolizumab, both given Q3W; or Arm 2c: 30 mg humanized antibody hCD27.131A in combination with 400 mg pembrolizumab, both given once every 6 weeks (Q6W).

Study treatment in Arms 1, 2, 3, 1a, 2a, and 2b was administered by IV infusion Q3W. Study treatment in Arm 2c was administered by IV infusion Q6W. The antibody hCD27.131A was administered over a period of approximately 90 minutes in all treatment arms. Based on preliminary safety information from the ongoing study, all participants treated with antibody hCD27.131A were prophylactically premedicated, 1.5 hours (±30 minutes) before infusion of antibody hCD27.131A with the following:
Diphenhydramine 50 mg orally (or equivalent dose of antihistamine); and
Acetaminophen 500-1000 mg orally (or equivalent dose of analgesic).

In Arms 2, 3, 2a, 2b, and 2c, antibody hCD27.131A was administered approximately 30 minutes after completion of the pembrolizumab infusion. The order of infusions in Arm 3 was as follows: pembrolizumab, antibody hCD27.131A, pemetrexed, then carboplatin. Participants should have received premedication per the approved product labels for pemetrexed and carboplatin.

TABLE 5
Study treatments

| | Study Treatment Name: | | | |
|---|---|---|---|---|
| | hCD27.131A antibody | | | |
| | (Arms 1, 2, 3, 1a, 2a, | Pembrolizumab [Arms | Pemetrexed | Carboplatin |
| | 2b, 2c) | 2, 3, 2a, 2b, 2c]) | (Arm 3) | (Arm 3) |
| Dose | Solution for | Solution for | Lyophilized | Solution for |
| Formulation: | infusion | infusion | powder for | infusion |
| | | | injection | |
| Unit Dose | 50 mg/mL, | 25 mg/mL, | 500 mg | 10 mg/mL, |
| Strength(s): | 1.0 mL vial | 4.0 mL vial | | 60 mL vial |
| Dose Level(s): | Arms 1 and 2: | Arms 2, 3, 2a, and 2b: | 500 mg/m² | AUC 5 |
| | Escalating doses based | 200 mg once every | once every 3 | mg/mL/min |
| | on pre-specified | 3 weeks | weeks | once every 3 |
| | dose-limiting toxicity | Arm 2c: 400 mg once | | weeks |
| | (DLT) criteria once | every 6 weeks | | |
| | every 3 weeks | | | |
| | Arms 3, 1a, 2a, 2b: 30 | | | |
| | mg once every 3 weeks | | | |
| | Arm 2c: 30 mg once | | | |
| | every 6 weeks | | | |
| Route of | IV infusion | IV infusion | IV infusion | IV infusion |
| Administration: | | | | |
| Sourcing: | Provided centrally | Provided centrally | Locally | Locally |
| | by the Sponsor | by the Sponsor | sourced | sourced |

In Arm 1a, antibody hCD27.131A and pembrolizumab were given for up to 35 cycles. In Arms 2a and 2b, antibody hCD27.131A were given for up to 6 months and pembrolizumab will be given for up to 35 cycles. In Arm 2c, antibody hCD27.131A were given for up to 6 months and pembrolizumab were given for up to 18 cycles.

Shown below is Table 6 with a listing of the objectives and endpoints for the male and female participants with advanced solid tumors who are at least 18 years of age who were included in the study:

TABLE 6
Objects and endpoints for the study

| Objectives | Endpoints |
|---|---|
| Objective: To determine the safety and | Number of participants with a DLT |
| tolerability of antibody hCD27.131A | Number of participants with ≥1 AE |
| in combination with pembrolizumab, | Number of participants who discontinue |
| pemetrexed, and carboplatin in | study treatment due to an AE |
| participants with non-squamous | |
| NSCLC and to establish maximum | |
| tolerated doses (MTDs) of carboplatin | |
| and pemetrexed when used in | |
| combination with antibody | |
| hCD27.131A and pembrolizumab | |

Tertiary/Exploratory

| | |
|---|---|
| Objective: To evaluate the preliminary | ORR and progression-free survival |
| anti-tumor activity of antibody | (PFS) as assessed by the investigator |
| hCD27.131A when used as | based on RECIST 1.1 and modified |
| monotherapy, in combination with | RECIST 1.1 for immune-based |
| pembrolizumab, and in combination | therapeutics (iRECIST) [Seymour, L., et |
| with pembrolizumab, pemetrexed, and | al 2017], and overall survival (OS) |
| carboplatin in the dose escalation and | |
| confirmation phase | |
| Objective: To evaluate the anti-tumor | ORR as assessed by the investigator |
| activity of antibody hCD27.131A | based on iRECIST, and PFS as assessed |
| when used as monotherapy and in | by the investigator based on |
| combination with pembrolizumab | RECIST 1.1 and iRECIST, and OS |
| in the dose expansion phase | |
| Objective: To evaluate development of | Circulating anti-antibody hCD27.131A |
| circulating anti-antibody hCD27.131A | and anti-pembrolizumab antibody levels |
| antibodies and anti-pembrolizumab | |
| antibodies, as appropriate, following | |
| administration of antibody | |
| hCD27.131A monotherapy, antibody | |
| hCD27.131A in combination with | |
| pembrolizumab, and antibody | |
| hCD27.131A in combination with | |
| pembrolizumab, pemetrexed, and | |
| carboplatin in the dose escalation and | |
| confirmation phase and/or the dose | |
| expansion phase | |
| Objective: To evaluate the PK of | PK parameters including AUC, |
| pembrolizumab when used in | Cₘᵢₙ, and Cₘₐₓ |
| combination with antibody | |
| hCD27.131A and the PK of | |
| pembrolizumab, pemetrexed, and | |
| carboplatin when used in combination | |
| with antibody hCD27.131A in the dose | |
| escalation and confirmation phase | |
| and/or the dose expansion phase | |
| Objective: To identify molecular | Blood- and/or tumor-derived |
| (genomic, metabolic, and/or proteomic) | biomarker parameters that may |
| biomarkers that may be indicative of | include but are not limited to DNA, |
| clinical response/resistance, safety, PD | RNA, protein, and metabolite-based |
| activity, and/or the mechanism of | analyses |
| action of hCD27.131A when used as | |
| monotherapy, in combination with | |
| pembrolizumab, and in combination | |
| with pembrolizumab, pemetrexed, and | |
| carboplatin in the dose escalation and | |
| confirmation phase and/or the dose | |
| expansion phase | |

Study Population

Male and female participants with advanced solid tumors who were at least 18 years of age on the day of signing consent were enrolled in this study.

Prospective approval of protocol deviations to recruitment and enrollment criteria, also known as protocol waivers or exemptions, was not permitted.

Inclusion Criteria

Participants were eligible to be included in the study only if all of the following criteria applied:

Type of Participant and Disease Characteristics

1. a) Dose Escalation/Confirmation Phase (Arms 1 and 2): Had a histologically or cytologically confirmed advanced/metastatic solid tumor by pathology report and had received or been intolerant to all treatment known to confer clinical benefit.

b) Dose Escalation/Confirmation Phase (Arm 3): Had a histologically or cytologically confirmed diagnosis of stage IV (M1a or M1b per current AJCC criteria, edition 8; see the AJCC Cancer Staging Manual, 8th Edition) non-squamous NSCLC.

Note: Mixed tumors were categorized by the predominant cell type; if small cell elements were present, the participant was ineligible.

Participants may have been untreated or could have received and progressed on 1 prior regimen.

Participants who have epidermal growth factor receptor (EGFR) or anaplastic lymphoma kinase (ALK) mutant tumors should have received an approved targeted therapy.

c) Dose Expansion Phase (Arm 2a): Had a diagnosis of TNBC. Participants must have received or been intolerant to not more than 2 lines of therapy for metastatic disease known to confer clinical benefit. Prior therapy should have included anthracycline and/or taxane for early-stage or metastatic disease. Participants must have had lactate dehydrogenase (LDH)≤2×ULN at screening. Enrollment was capped at a maximum of 7-10 participants who are PD-1/PD-L1 inhibitor treatment-refractory*.

d) Dose Expansion Phase (Arm 1a, 2b, and 2c): Had a diagnosis of endometrial cancer. Participants must have received or been intolerant to no more than 2 prior lines of treatment known to confer clinical benefit. Prior therapy should have included:

platinum-containing regimens for early-stage or metastatic disease. Enrollment was be capped at a maximum of 5-7 participants per treatment arm who were PD-1/PD-L1 inhibitor treatment-refractory*. *Participants were considered to have PD-1/PD-L1 inhibitor treatment-refractory disease if they met all of the following criteria:

i. Had received at least 2 doses of anti-PD-1/PD-L1 mAb at a local regulatory agency-approved dose and schedule.

ii. Had progressive disease after anti-PD-1/PD-L1 mAB defined according to RECIST 1.1. The initial evidence of progressive disease was to be confirmed by a second assessment no less than 4 weeks from the date of the first documented progressive disease, in the absence of rapid clinical progression.

iii. Had documented disease progression within 12 weeks of the last dose of

anti-PD-1/PD-L1 mAb. Participants who were re-treated with anti-PD-1/PD-L1 mAb and participants who were on maintenance with anti-PD-1/PD-L1 mAb were not be considered to have PD-1/PD-L1 inhibitor treatment-refractory disease if more than 12 weeks elapsed between disease progression and the last treatment date (with anti-PD1/PD-L1 therapy).

2. Had measurable disease by RECIST 1.1. as assessed by the local site investigator/radiologist. Target lesions situated in a previously irradiated area were to be considered measurable if progression has been demonstrated in such lesions.

3. Had adequate organ function. Specimens were to have been collected within 7 days before the first dose of study treatment.

Exclusion Criteria

Participants were excluded from the study if any of the following criteria apply:

Medical Conditions

1. Had a history of a second malignancy, unless potentially curative treatment had been completed with no evidence of malignancy for 2 years.

Note: The time requirement was not to apply to the disease under study, participants who underwent successful definitive resection of basal cell carcinoma of the skin, squamous cell carcinoma of the skin, superficial bladder cancer, in situ cervical cancer, or other in situ cancers.

2. Had clinically active central nervous system (CNS) metastases and/or carcinomatous meningitis. Participants with previously-treated brain or meningeal metastases may participated and have been eligible for treatment provided they were stable and asymptomatic (without evidence of progression by magnetic resonance imaging [MRI] scan of the brain separated by at least 4 weeks after treatment), have had no evidence of new or enlarging brain metastases, were evaluated within 4 weeks before the start of study treatment, and were off immunosuppressive doses of systemic steroids for at least 2 weeks prior to enrollment.

3. Have had a severe hypersensitivity reaction to treatment with a mAb and/or other components of the study treatment.

4. Had an active infection requiring systemic treatment.

5. Had a history of interstitial lung disease.

6. Had a history of (noninfectious) pneumonitis that required steroids or current pneumonitis.

7. Had symptomatic ascites or pleural effusion. Participants who were clinically stable after treatment of these conditions (including therapeutic thoracocentesis or paracentesis) were not be excluded from participation in this study.

8. Had previously had a stem cell or bone marrow transplant.

9. Had previously had a solid organ transplant.

10. Had an active autoimmune disease that has required systemic treatment in the past 2 years (i.e., with use of disease-modifying agents, corticosteroids, or immunosuppressive drugs) except vitiligo or resolved childhood asthma/atopy. Replacement therapy, such as thyroxine, insulin, or physiologic corticosteroid replacement therapy for adrenal or pituitary insufficiency, was not considered a form of systemic treatment and was allowed. Use of non-systemic steroids was permitted.

11. Had known human immunodeficiency virus ([HIV]; HIV 1 or 2 antibodies) and/or active and acute Hepatitis B or C infections (e.g., positive for HBsAg/HBV DNA or HCV RNA).

12. Had a history or current evidence of any condition, therapy, or laboratory abnormality that might confound the results of the study, interfere with the participant's participation for the full duration of the study, make administration of the study treatments hazardous, or make it difficult to monitor adverse effects such that it is not in the best interest of the participant to participate, in the opinion of the treating investigator.

13. Had not fully recovered from any effects of major surgery without significant detectable infection. Surgeries that required general anesthesia must have been completed at least 2 weeks before the start of study treatment. Surgeries that required regional/epidural anesthesia must have been completed at least 72 hours before the first dose of study treatment and participants should have recovered.

14. Had known psychiatric or substance abuse disorders that would interfere with the participant's ability to cooperate with the requirements of the study.

15. Was pregnant or breastfeeding, or expecting to conceive or father children within the projected duration of the study.

16. A WOCBP who had a positive urine pregnancy test within 72 hours before the first dose of study treatment. If the urine test was positive or could not be confirmed as negative, a serum pregnancy test was required. In such cases, the participant would have been excluded from participation if the serum pregnancy test result was positive. Note, in the event that 72 hours had elapsed between the screening pregnancy test and the first dose of study treatment, another pregnancy test (urine or serum) would have been performed and had to be negative in order for the participant to start receiving study treatment.

Prior/Concomitant Therapy

17. Has had chemotherapy, definitive radiation, or biological cancer therapy within 4 weeks (2 weeks for palliative radiation) before the first dose of study treatment, or had not recovered to CTCAE Grade ≤1 or better from any AEs that were due to cancer therapeutics administered more than 4 weeks earlier (this included participants with previous immunomodulatory therapy with residual immune-related AEs [irAEs]). Participants receiving ongoing replacement hormone therapy for endocrine irAEs were not to be excluded from participation in this study.

18. Was expected to require any other form of antineoplastic therapy while participating in this study.

19. Had received previous treatment with another agent targeting CD27.

20. Had received prior therapy with an agent directed to another stimulatory T cell receptor (e.g., OX-40, CD137).

21. Was on chronic systemic steroid therapy in excess of replacement doses (e.g., exceeding 10 mg/day of prednisone equivalent), or on any other form of immunosuppressive medication. Participants with reactive airway disease that require intermittent use of bronchodilators, inhaled steroids, or local steroid injections were not to be excluded from this study.

22. Was a regular user (including “recreational use”) of any illicit drugs at the time of signing informed consent, or had a recent history (within the last year) of substance abuse (including alcohol), as determined by the treating investigator.

23. Had received a live-virus vaccine within 28 days before the first dose of study treatment. Examples of live vaccines include, but were not limited to, the following: measles, mumps, rubella, varicella/zoster (chicken pox), yellow fever, rabies, Bacillus Calmette-Guérin (BCG), and typhoid vaccine. Seasonal flu vaccines that do not contain live viruses were permitted. Intranasal influenza vaccines (e.g., FluMist®) as live attenuated vaccines and were not permitted.

Prior/Concurrent Clinical Study Experience

24. Was currently participating and receiving study therapy in a study of an investigational agent or had participated and received study therapy in a study of an investigational agent or had used an investigational device within 28 days before the first dose of study treatment.

Note: Participants who had entered the follow-up phase of an investigational study could participate as long as it had been 4 weeks since the last dose of the previous investigational agent.

Additional Exclusion Criteria for Participants Treated in Arm 3:

25. Had received radiation therapy to the lung that was >30 Gy within 6 months before the first dose of study treatment.

26. Was unable to interrupt aspirin or other nonsteroidal anti-inflammatory drugs (NSAIDs), other than an aspirin dose ≤1.3 g per day, for a 5-day period (8-day period for long-acting agents, such as piroxicam).

27. Was unable or unwilling to take folic acid or vitamin B12 supplementation.

Discussion/Results

Key eligibility criteria included histological- or cytologically-confirmed advanced solid tumor, measurable disease by RECIST v1.1, and ECOG PS ≤1. As discussed above, antibody hCD27.131A was tested alone (dose range, 2-700 mg) or with pembrolizumab (fixed dose, 200 mg). Patients with disease progression following antibody hCD27.131A monotherapy were eligible to crossover to combination treatment. The primary objective was safety and tolerability. Objective response rate by investigator per RECIST v1.1 was also evaluated.

### 44 Patient Analysis

Of the 44 patients enrolled initially, 25 were administered antibody hCD27.131A and 19 were administered antibody hCD27.131A plus pembrolizumab. Data showed that the median age was 59.0 years and 61.4% of those patients were female, 47.7% had ECOG PS 1, and 13.6% received prior immune checkpoint inhibitor therapy. See Table 7A and Table 7B.

TABLE 7A
Baseline characteristics

| | | | hCD27.131A + | | | |
|---|---|---|---|---|---|---|
| | hCD27.131A | | Pembro | | Total | |
| Characteristic, n (%) | n = 25 | | n = 19 | | N = 44 | |
| Age, median years | 60.0 | (28-77) | 59.0 | (23-73) | 59.0 | (23-77) |
| (range) | | | | | | |
| Male | 11 | (44.0) | 6 | (31.6) | 17 | (38.6) |
| White | 25 | (100) | 19 | (100) | 44 | (100) |

ECOG performance status

| | | | | | | |
|---|---|---|---|---|---|---|
| 0 | 13 | (52.0) | 10 | (52.6) | 23 | (52.3) |
| 1 | 12 | (48.0) | 9 | (47.4) | 21 | (47.7) |
| Received prior | 2 | (8.0) | 4 | (21.1) | 6 | (13.6) |
| immune | | | | | | |
| checkpoint | | | | | | |
| inhibitor | | | | | | |

TABLE 7B
Baseline characteristics CONT'D

| | | | hCD27.131A + | | | |
|---|---|---|---|---|---|---|
| | hCD27.131A | | Pembro | | Total | |
| Characteristic, n (%) | n = 25 | | n= 19 | | N = 44 | |
| Primary diagnosis^{a} | | | | | | |
| Ovarian | 1 | (4.0) | 6 | (31.6) | 7 | (15.9) |
| CRC | 3 | (12.0) | 3 | (15.8) | 6 | (13.6) |
| STS | 3 | (12.0) | 3 | (15.8) | 6 | (13.6) |
| Breast | 3 | (12.0) | 1 | (5.3) | 4 | (9.1) |
| GI | 2 | (8.0) | 1 | (5.3) | 3 | (6.8) |
| Cervical | 2 | (8.0) | 0 | | 2 | (4.5) |
| Endometrial | 1 | (4.0) | 1 | (5.3) | 2 | (4.5) |
| Head and neck | 2 | (8.0) | 0 | | 2 | (4.5) |
| NSCLC | 0 | | 2 | (10.5) | 2 | (4.5) |
| Pancreatic | 1 | (4.0) | 1 | (5.3) | 2 | (4.5) |

| | | | | | |
|---|---|---|---|---|---|
| Prostate | 2 | (8.0) | 0 | 2 | (4.5) |

^{a}GU, liver, mesothelioma, and skin cancer were reported in 1 patient each in the hCD27.131A arm; kidney cancer was reported in 1 patient in the hCD27.131A + Pembro arm.

In the initial phase, dose-limiting toxicities (DLT) were reported in 3 patients receiving antibody hCD27.131A and 1 patient receiving antibody hCD27.131A plus pembrolizumab. See Table 8. All the DLTs observed were associated with infusion-related adverse events. Maximum-tolerated dose was defined. Treatment-related adverse events (TRAEs) were reported in 40 patients (90.9%), 22 patients (88.0%) receiving antibody hCD27.131A and 18 patients (94.7%) receiving antibody hCD27.131A plus pembrolizumab. See Table 8. The most common TRAEs were fatigue (28.0%) and infusion-related reactions (28.0%) with treatment with antibody hCD27.131A and fatigue (36.8%) and pruritus (31.6%) with a combination treatment of antibody hCD27.131A and pembrolizumab. See Table 9. Grade 3-4 TRAEs were reported in 10 patients (22.7%); the TRAEs were reported in 6 patients (24.0%) receiving antibody hCD27.131A and 4 patients (21.1%) receiving antibody hCD27.131A plus pembrolizumab. See Table 10. No grade 5 events were observed. Data show that there were confirmed responses. One patient (4.0%) achieved a partial response (PR) with antibody hCD27.131A and 1 patient (5.3%) achieved a PR with antibody hCD27.131A plus pembrolizumab. A summary of best overall response is shown in Table 12. It was observed that patients did show an anti-tumor response to the treatments. The duration of response is shown in Table 11.

There were 14 patients who entered the crossover phase to receive a combination therapy including the antibody hCD27.131A and the antibody pembrolizumab. In the crossover phase, no DLTs were reported. TRAEs were reported in 12 patients (85.7%). The most common TRAEs were pruritus (21.4%), rash (21.4%), and headache (14.3%). One patient (7.1%) reported grade 3-4 TRAEs of increased amylase and increased lipase. No grade 5 events were observed. Two patients (14.3%) achieved a complete response and 2 patients (14.3%) achieved a PR.

Thus, the initial data shows that treatment with antibody hCD27.131A, alone and in combination with pembrolizumab, demonstrated an acceptable safety profile. Early antitumor activity was observed in patients with advanced solid tumors in both monotherapy and combination therapy arms.

TABLE 8
Summary of Treatment-Related Adverse Events^{a}

| | | hCD27.131A + | | |
|---|---|---|---|---|
| | hCD27.131A | Pembro | | Total |
| n (%) | n = 25 | n = 19 | | N = 44 |
| Any TRAE | 22 (88.0) | 18 | (94.7) | 40 (90.9) |
| Grade 3-4 TRAE | 6 (24.0) | 4 | (21.1) | 10 (22.7) |
| Serious TRAE | 0 | 2 | (10.5) | 2 (4.5) |
| Death due to TRAE | 0 | 0 | | 0 |
| Discontinuation due to | 1 (4.0) | 2 | (10.5) | 3 (6.8) |
| TRAE | | | | |
| Discontinuation due to | 0 | 1 | (5.3) | 1 (2.3) |
| treatment-related SAE | | | | |
| DLTs | 3 (12.0) | 1 | (5.3) | 4 (9.1) |

^{a}Determined by investigator to be related to the drug.

TABLE 9
Treatment-Related Adverse Events (≥10% in any arm)^{a}

| | | | hCD27.131A + | | |
|---|---|---|---|---|---|
| | hCD27.131A | | Pembro | Total | |
| TRAE, n (%) | n = 25 | | n = 19 | N = 44 | |
| Fatigue | 7 | (28.0) | 7 (36.8) | 14 | (31.8) |
| Infusion-related | 7 | (28.0) | 4 (21.1) | 11 | (25.0) |
| reaction | | | | | |
| Nausea | 5 | (20.0) | 5 (26.3) | 10 | (22.7) |
| Pruritus | 4 | (16.0) | 6 (31.6) | 10 | (22.7) |
| Rash | 4 | (16.0) | 3 (15.8) | 7 | (15.9) |
| Pyrexia | 2 | (8.0) | 4 (21.1) | 6 | (13.6) |
| Myalgia | 3 | (12.0) | 2 (10.5) | 5 | (11.4) |
| Chills | 2 | (8.0) | 2 (10.5) | 4 | (9.1) |
| Diarrhea | 2 | (8.0) | 2 (10.5) | 4 | (9.1) |
| Dry mouth | 1 | (4.0) | 2 (10.5) | 3 | (6.8) |
| Influenza-like illness | 1 | (4.0) | 2 (10.5) | 3 | (6.8) |
| Vomiting | 3 | (12.0) | 0 | 3 | (6.8) |

| | | | | |
|---|---|---|---|---|
| Increased amylase | 0 | 2 (10.5) | 2 | (4.5) |

^{a}Events per MedDRA preferred terms and determined by investigator to be related to the drug.

TABLE 10
Grade 3-4 Treatment-Related Adverse Events^{a}

| | | hCD27.131A + | |
|---|---|---|---|
| | hCD27.131A | Pembro | Total |
| TRAE, n (%) | n = 25 | n = 19 | N = 44 |
| Amylase increased | 0 | 1 (5.3) | 1 (2.3) |
| Aspartate | 1 (4.0) | 0 | 1 (2.3) |
| aminotransferase | | | |
| increased | | | |
| Decreased appetite | 1 (4.0) | 0 | 1 (2.3) |
| Hypophosphatemia | 0 | 1 (5.3) | 1 (2.3) |
| Hypotension | 0 | 1 (5.3) | 1 (2.3) |
| Hypoxia | 1 (4.0) | 0 | 1 (2.3) |
| Infusion-related | 1 (4.0) | 0 | 1 (2.3) |
| reaction | | | |
| Lipase increased | 1 (4.0) | 0 | 1 (2.3) |
| Pruritus | 1 (4.0) | 0 | 1 (2.3) |
| Tumor pain | 0 | 1 (5.3) | 1 (2.3) |

^{a}Events per MedDRA preferred terms and determined by investigator to be related to the drug.

TABLE 11
Duration of Response ^{a}

| | | | hCD27.131A + |
|---|---|---|---|
| | | hCD27.131A + | Pembro, |
| | hCD27.131A | Pembro | Crossover |
| | n = 25 | n = 19 | n = 14 |
| Patients with | 1 | 1 | 4 |
| response, n | | | |
| Median time to | 2.0 (2.0-2.0) | 1.8 (1.8-1.8) | 2.1 (1.9-4.2) |
| response, months | | | |
| (range) | | | |
| Median duration of | 4.1 (4.1-4.1) | 5.0 (5.0-5.0) | NR (2.2+-6.5+)^{b} |
| response, months | | | |
| (range) | | | |
| Patients with | | | |
| extended | | | |
| response duration, n | | | |
| ≥3 months | 1 | 1 | 3 |
| ≥6 months | 0 | 0 | 2 |

^{a} Based on investigator assessment per RECIST v1.1 in patients with confirmed response.
^{b}“+” indicates there was no progressive disease at the time of last disease assessment.

TABLE 12
Summary of Best Overall Response^{a}

| | | | | hCD27.131A + |
|---|---|---|---|---|
| | | | hCD27.131A + | Pembro, |
| | hCD27.131A | | Pembro | Crossover |
| n (%) | n = 25 | | n = 19 | n = 14 |
| Objective response | 1 | (4.0) | 2 (10.5) | 5 (35.7) |
| (CR + PR) | | | | |
| CR | 0 | | 0 | 2 (14.3) |
| PR | 1 | (4.0) | 2 (10.5) | 3 (21.4) |
| SD | 5 | (20.0) | 7 (36.8) | 1 (7.1) |
| DCR (CR + PR + SD ≥ | 2 | (8.0) | 2 (10.5) | 5 (35.7) |
| 12 mo) | | | | |
| Progressive disease (PD) | 16 | (64.0) | 7 (36.8) | 6 (42.9) |
| No assessment^{b} | 3 | (12.0) | 3 (15.8) | 2 (14.3) |

^{a}Based on investigator assessment per RECIST v1.1 without response confirmation. All responses were confirmed except for 1 patient in the hCD27.131A + Pembro arm who was classified as having a PR which was later confirmed as SD and 1 patient in the hCD27.131A + Pembro crossover arm who was classified as having a PR which was later confirmed as SD.
^{b}Includes patients without post-baseline assessment on the data cutoff date.

Efficacy, Response and Anti-Tumor Activity

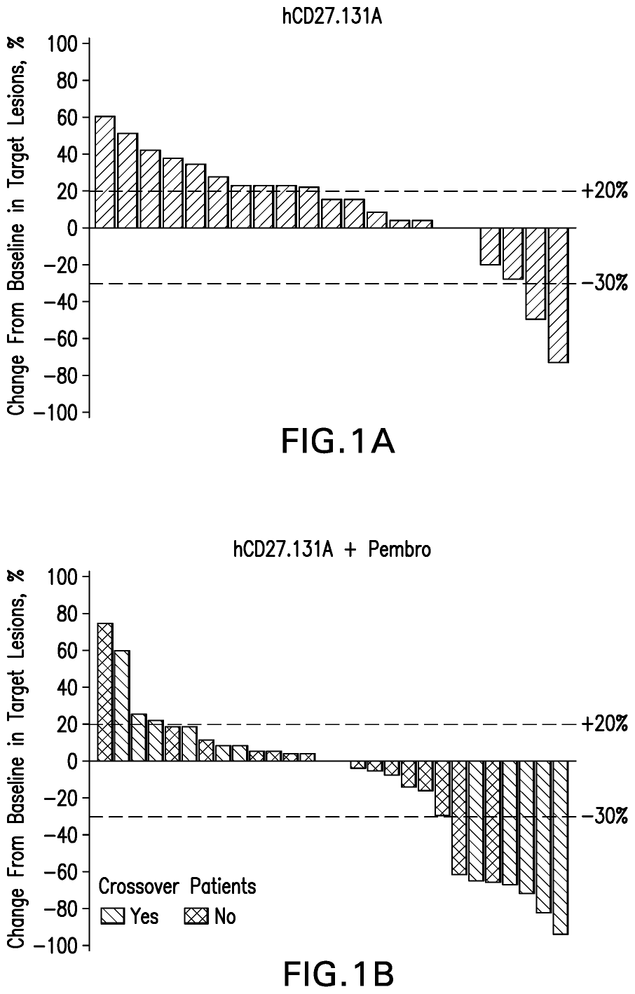

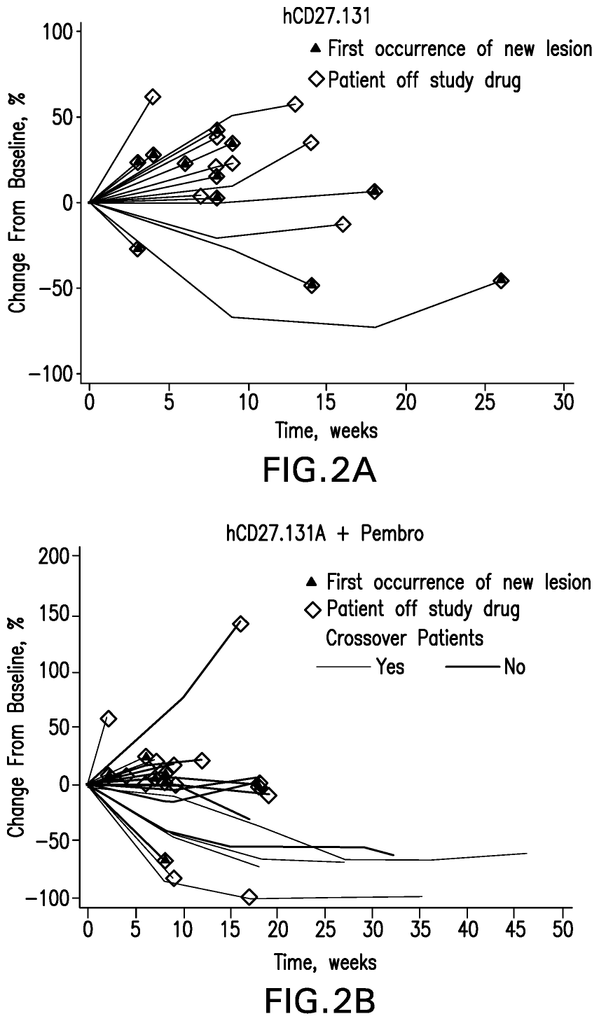

Efficacy and activity of the hCD27.131A antibody (as a monotherapy or as a combination therapy with pembrolizumab) were analyzed. Early antitumor activity was observed in patients with advanced solid tumors in both the monotherapy and the combination therapy arms. FIG. 1A and FIG. 1B show the best percentage change from baseline in target lesions. See also FIG. 2A and FIG. 2B. A duration of response and a summary of best overall response is shown in Table 11 and Table 12. It was observed that patients did show a response to the monotherapy and combination treatments described herein. Data show 1 patient achieved confirmed PR when treated with antibody hCD27.131A, one patient achieved confirmed PR with a combination treatment with antibody hCD27.131A pembrolizumab, and in the crossover phase, two patients achieved confirmed CR and 2 patients achieved confirmed PR with a combination treatment with antibody hCD27.131A and pembrolizumab.

Pharmacodynamics (PD) and Pharmacokinetics (PK)

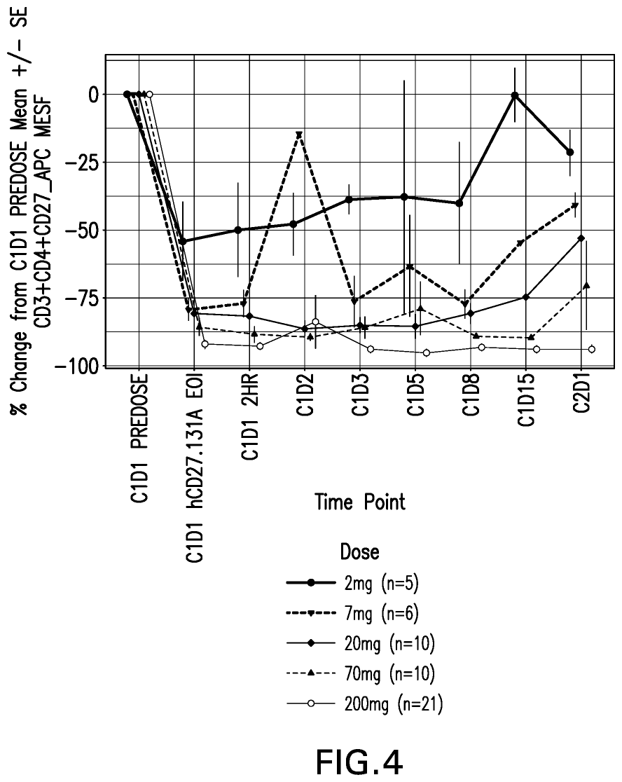

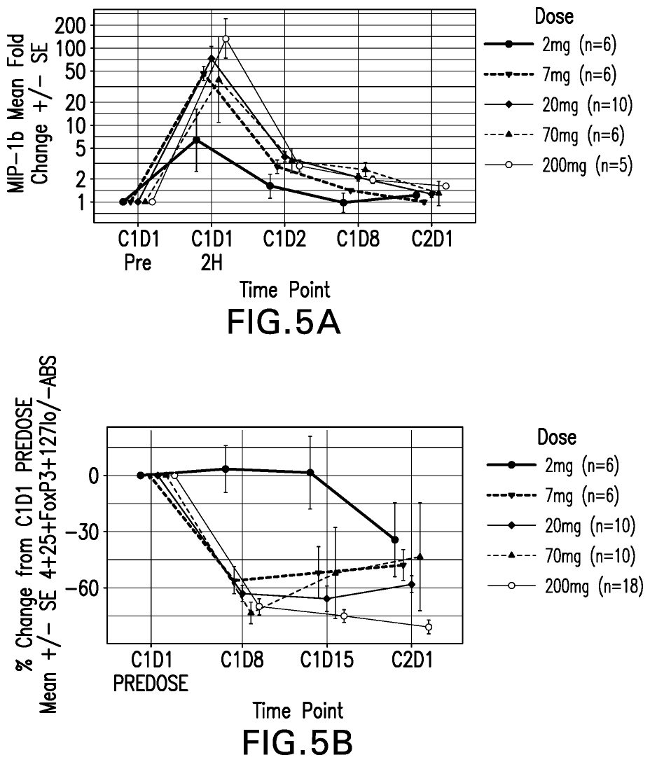

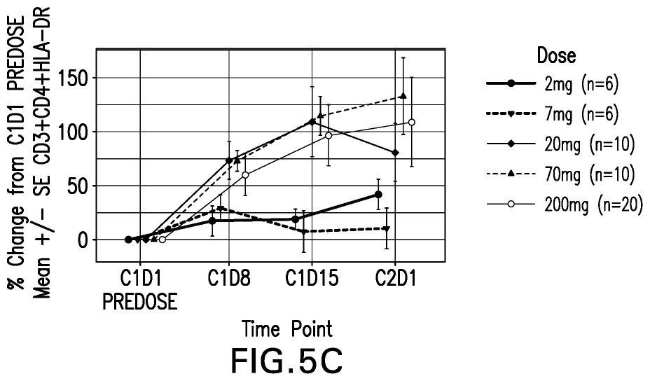

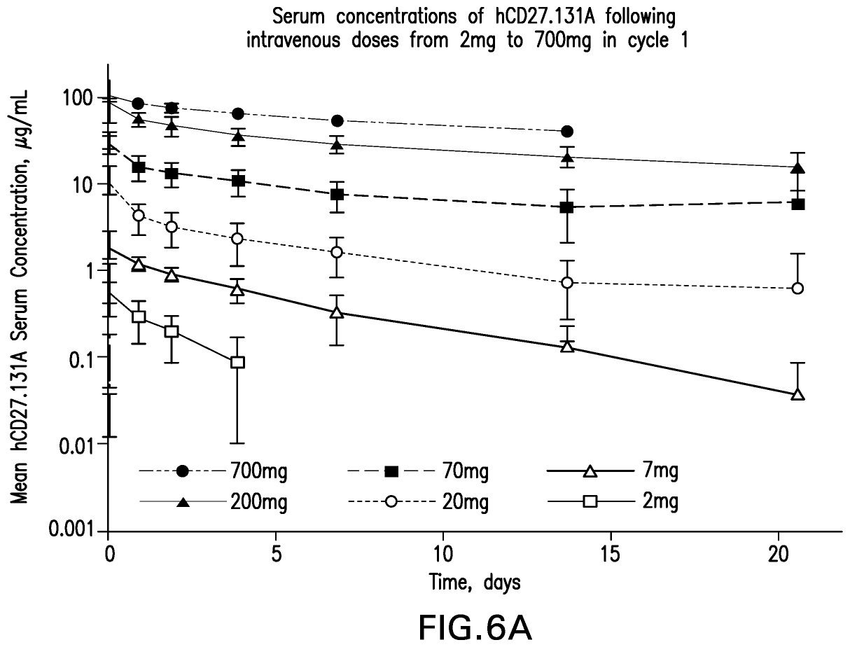

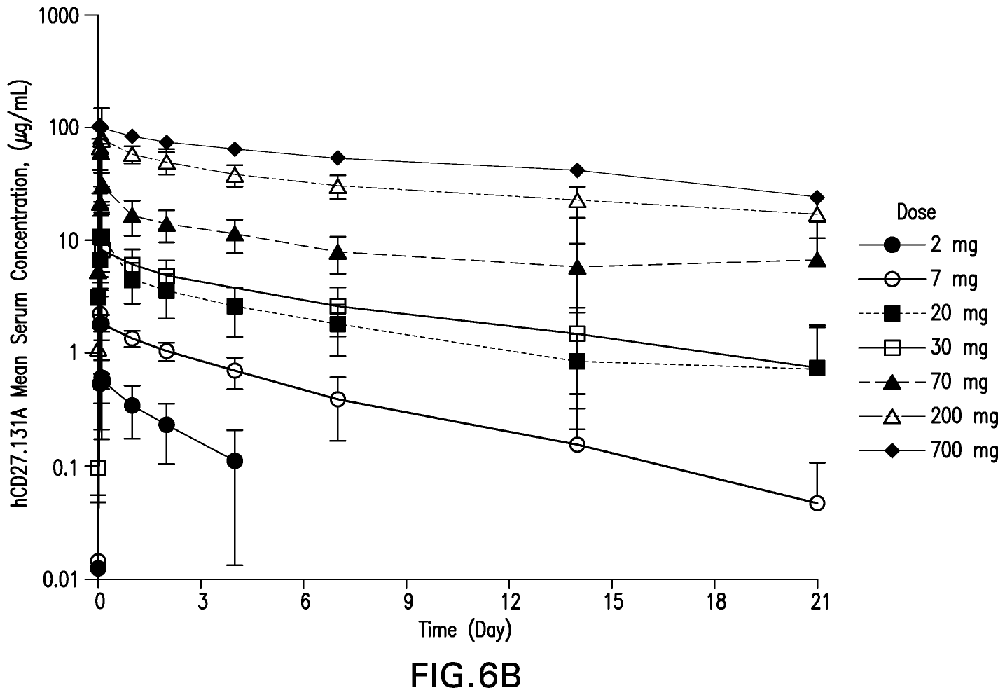

Serum and blood samples from the subjects treated with antibody hCD27.131A were subsequently analyzed for PD and PK of the antibody during cycle 1. Data show dose proportional decreases in CD27 receptor availability on T-cells in blood with increasing antibody doses generally approaching target saturation at ≥200 mg. See also receptor availability data shown in FIG. 4. It was also observed that preliminary PD profiles of antibody hCD27.131A elicited on-treatment immune effects in serum and blood including: transient increases in chemotactic chemokine MIP-1β (FIG. 5A); decreases in T-cell subsets including regulatory T-cells (FIG. 5B); and increases in the frequency of HLA-DR+ activated T-cells (FIG. 5C). FIG. 6A shows the serum concentrations of antibody hCD27.131A following intravenous doses of the antibody from 2 mg to 700 mg in cycle 1. See also FIG. 6B. The half-life (T½) calculated in the PK analysis ranged from 4 days at 20 mg to 15 days at 200 mg in cycle 1. Preliminary PK profiles of antibody hCD27.131A exposures may suggest that target-mediated clearance of the antibody is saturated at 200 mg.

### SUMMARY

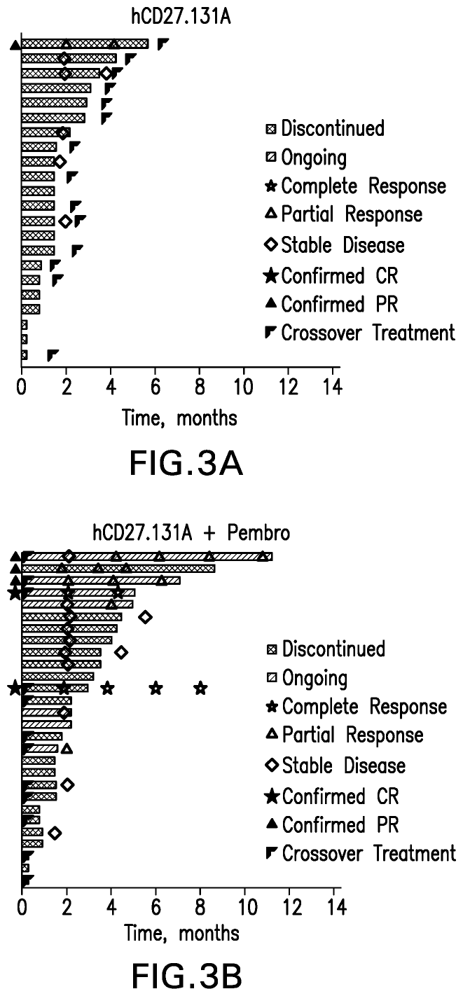

In conclusion, it was observed that antibody hCD27.131A monotherapy and combination treatment with antibody hCD27.131A and pembrolizumab were well tolerated. Data show acceptable pharmacodynamics and pharmacokinetics, and also effective anti-tumor activity in patients having advanced solid tumor types and treated with either antibody hCD27.131A alone or a combination therapy of hCD27.131A and pembrolizumab. Specific tumor responses were observed for patients treated with antibody hCD27.131A monotherapy alone and also in patients treated with the combination therapy of antibody hCD27.131A and pembrolizumab. Responses were observed in patients who crossed over from monotherapy to combination therapy. FIG. 1A and FIG. 1B and FIG. 2A and FIG. 2B set forth the best percentage change from baseline in target lesions (RECIST v1.1, Investigator Review). FIG. 3A and FIG. 3B set forth a diagram of the treatment duration and response (RECIST v1.1, Investigator Review). A summary of the anti-tumor activity (RECIST v1.1, Investigator Review) is set forth below in Table 12. See also Table 11.

### 120 Patient Data Analysis

At the data cutoff date 12 Jun. 2020, 120 patients enrolled of which: 36 subjects were administered humanized antibody hCD27.131A at dose levels from 2 to 700 mg given Q3W as monotherapy; 78 subjects were administered humanized antibody hCD27.131A at dose levels from 2 to 200 mg in combination with: 1) 200 mg pembrolizumab Q3W, or 2) with 400 mg pembrolizumab Q6W; 6 subjects were administered 30 mg of hCD27.131A antibody Q3W, pembrolizumab (200 mg, Q3W) and chemotherapy; and 20 subjects were administered hCD27.131A antibody at different dose levels (depending on the DLT clearance for particular dose at the time of crossover) Q3W, pembrolizumab (200 mg, Q3W) in the combination therapy CROSSOVER. Data for these patients was collected and analyzed, e.g., 8.3% received prior immune checkpoint inhibitor therapy (Table 13). As mentioned above twenty patients crossed over to combination therapy with pembrolizumab. Primary diagnosis for patients is listed in Table 14.

TABLE 13
Subject Characteristics (ASaT Population in Initial Treatment Phase)

| | | | | | | | hCD27.131A + | | | |
|---|---|---|---|---|---|---|---|---|---|---|
| | | | | | hCD27.131A | | Pembro 200 | | | |
| | | | hCD27.131A + | | 30 mg Q3W + | | mg Q3W | | | |
| | hCD27.131A | | Pembro | | Pembro 200 | | Combination | | | |
| | Q3W | | Combination | | mg Q3W + | | Therapy | | | |
| | Monotherapy | | Therapy | | Chemotherapy | | CROSSOVER | | Total | |
| | n | (%) | n | (%) | n | (%) | n | (%) | n | (%) |
| Subjects in population | 36 | | 78 | | 6 | | 20 | | 120 | |

Gender

| | | | | | | | | | | |
|---|---|---|---|---|---|---|---|---|---|---|
| Male | 11 | (30.6) | 10 | (12.8) | 2 | (33.3) | 7 | (35.0) | 23 | (19.2) |
| Female | 25 | (69.4) | 68 | (87.2) | 4 | (66.7) | 13 | (65.0) | 97 | (80.8) |

Age (Years)

| | | | | | | | | | | |
|---|---|---|---|---|---|---|---|---|---|---|
| <65 | 15 | (41.7) | 54 | (69.2) | 5 | (83.3) | 7 | (35.0) | 74 | (61.7) |
| >=65 | 21 | (58.3) | 24 | (30.8) | 1 | (16.7) | 13 | (65.0) | 46 | (38.3) |
| Mean | 62.6 | | 56.5 | | 63.3 | | 61.6 | | 59.1 | |
| SD | 14.0 | | 13.7 | | 7.0 | | 14.4 | | 13.8 | |
| Median | 66.0 | | 58.0 | | 61.0 | | 66.5 | | 61.5 | |
| Range | 28 to | | 23 to | | 58 to | | 28 to | | 23 to | |
| | 85 | | 88 | | 77 | | 77 | | 88 | |

Race

| | | | | | | | | | | |
|---|---|---|---|---|---|---|---|---|---|---|
| Asian | 0 | (0.0) | 1 | (1.3) | 0 | (0.0) | 0 | (0.0) | 1 | (0.8) |
| Black Or African American | 0 | (0.0) | 1 | (1.3) | 0 | (0.0) | 0 | (0.0) | 1 | (0.8) |
| White | 34 | (94.4) | 76 | (97.4) | 6 | (100.0) | 18 | (90.0) | 116 | (96.7) |
| Missing | 2 | (5.6) | 0 | (0.0) | 0 | (0.0) | 2 | (10.0) | 2 | (1.7) |

Ethnicity

| | | | | | | | | | | |
|---|---|---|---|---|---|---|---|---|---|---|
| Hispanic Or Latino | 1 | (2.8) | 2 | (2.6) | 0 | (0.0) | 1 | (5.0) | 3 | (2.5) |
| Not Hispanic Or Latino | 33 | (91.7) | 72 | (92.3) | 5 | (83.3) | 17 | (85.0) | 110 | (91.7) |
| Not Reported | 0 | (0.0) | 4 | (5.1) | 1 | (16.7) | 0 | (0.0) | 5 | (4.2) |
| Unknown | 2 | (5.6) | 0 | (0.0) | 0 | (0.0) | 2 | (10.0) | 2 | (1.7) |

Receiving prior immune checkpoint inhibitor

| | | | | | | | | | | |
|---|---|---|---|---|---|---|---|---|---|---|
| Y | 2 | (5.6) | 8 | (10.3) | 0 | (0.0) | 0 | (0.0) | 10 | (8.3) |
| N | 34 | (94.4) | 70 | (89.7) | 6 | (100.0) | 20 | (100.0) | 110 | (91.7) |

ECOG

| | | | | | | | | | | |
|---|---|---|---|---|---|---|---|---|---|---|
| 0 | 19 | (52.8) | 50 | (64.1) | 1 | (16.7) | 14 | (70.0) | 70 | (58.3) |
| 1 | 17 | (47.2) | 28 | (35.9) | 5 | (83.3) | 6 | (30.0) | 50 | (41.7) |

TABLE 14
Subject Characteristics (ASaT Population in Initial Treatment Phase)

| | | | | | | hCD27.131A + | | | |
|---|---|---|---|---|---|---|---|---|---|
| | | | | hCD27.131A | | Pembro 200 | | | |
| | | hCD27.131A + | | 30 mg Q3W + | | mg Q3W | | | |
| hCD27.131A | | Pembro | | Pembro 200 | | Combination | | | |
| Q3W | | Combination | | mg Q3W + | | Therapy | | | |
| Monotherapy | | Therapy | | Chemotherapy | | CROSSOVER | | Total | |
| n | (%) | n | (%) | n | (%) | n | (%) | n | (%) |

Primary Diagnosis

| | | | | | | | | | | |
|---|---|---|---|---|---|---|---|---|---|---|
| Breast Cancer (Nos) | 3 | (8.3) | 25 | (32.1) | 0 | (0.0) | 2 | (10.0) | 28 | (23.3) |
| Carcinoma Of Gastro- | 1 | (2.8) | 0 | (0.0) | 0 | (0.0) | 1 | (5.0) | 1 | (0.8) |
| Esophageal Junction | | | | | | | | | | |
| Carcinoma, Nos | 0 | (0.0) | 2 | (2.6) | 0 | (0.0) | 0 | (0.0) | 2 | (1.7) |
| Cervical Cancer, Nos | 2 | (5.6) | 0 | (0.0) | 0 | (0.0) | 1 | (5.0) | 2 | (1.7) |
| Cholangio Carcinoma | 0 | (0.0) | 1 | (1.3) | 0 | (0.0) | 0 | (0.0) | 1 | (0.8) |
| Colorectal Cancer, Nos | 3 | (8.3) | 2 | (2.6) | 0 | (0.0) | 0 | (0.0) | 5 | (4.2) |
| Duodenum Cancer | 1 | (2.8) | 0 | (0.0) | 0 | (0.0) | 0 | (0.0) | 1 | (0.8) |
| Endometrial Adenocarcinoma | 0 | (0.0) | 1 | (1.3) | 0 | (0.0) | 0 | (0.0) | 1 | (0.8) |
| Endometrial Cancer, Nos | 7 | (19.4) | 17 | (21.8) | 0 | (0.0) | 4 | (20.0) | 24 | (20.0) |
| Endometrial Serous | 0 | (0.0) | 1 | (1.3) | 0 | (0.0) | 0 | (0.0) | 1 | (0.8) |
| Carcinoma | | | | | | | | | | |
| Endometroid | 0 | (0.0) | 1 | (1.3) | 0 | (0.0) | 0 | (0.0) | 1 | (0.8) |
| Adenocarcinoma | | | | | | | | | | |
| Esophageal Cancer, Nos | 0 | (0.0) | 1 | (1.3) | 0 | (0.0) | 0 | (0.0) | 1 | (0.8) |
| Extraskeletal Myxoid | 0 | (0.0) | 1 | (1.3) | 0 | (0.0) | 0 | (0.0) | 1 | (0.8) |
| Chondrosarcoma | | | | | | | | | | |
| Head & Neck Cancer, Nos | 1 | (2.8) | 1 | (1.3) | 0 | (0.0) | 0 | (0.0) | 2 | (1.7) |
| Kidney Cancer, Nos | 0 | (0.0) | 1 | (1.3) | 0 | (0.0) | 0 | (0.0) | 1 | (0.8) |
| Liver/Hepatobiliary Cancer | 1 | (2.8) | 0 | (0.0) | 0 | (0.0) | 1 | (5.0) | 1 | (0.8) |
| Melanoma | 0 | (0.0) | 1 | (1.3) | 0 | (0.0) | 0 | (0.0) | 1 | (0.8) |
| Mesothelioma | 1 | (2.8) | 0 | (0.0) | 0 | (0.0) | 1 | (5.0) | 1 | (0.8) |
| Nsclc, Nos | 0 | (0.0) | 2 | (2.6) | 5 | (83.3) | 0 | (0.0) | 7 | (5.8) |
| Osteosarcoma | 0 | (0.0) | 1 | (1.3) | 0 | (0.0) | 0 | (0.0) | 1 | (0.8) |
| Ovarian Cancer | 1 | (2.8) | 9 | (11.5) | 0 | (0.0) | 1 | (5.0) | 10 | (8.3) |
| Pancreatic Cancer (Not Islets) | 1 | (2.8) | 1 | (1.3) | 0 | (0.0) | 1 | (5.0) | 2 | (1.7) |
| Pleural Mesothelioma | 0 | (0.0) | 1 | (1.3) | 0 | (0.0) | 0 | (0.0) | 1 | (0.8) |
| Prostate Cancer, Nos | 2 | (5.6) | 0 | (0.0) | 0 | (0.0) | 1 | (5.0) | 2 | (1.7) |
| Sarcoma | 1 | (2.8) | 1 | (1.3) | 0 | (0.0) | 0 | (0.0) | 2 | (1.7) |
| Serous Carcinoma | 0 | (0.0) | 1 | (1.3) | 0 | (0.0) | 0 | (0.0) | 1 | (0.8) |
| Serous Carcinoma With Focal | 1 | (2.8) | 0 | (0.0) | 0 | (0.0) | 1 | (5.0) | 1 | (0.8) |
| Clear Cell Carcinoma | | | | | | | | | | |
| Serous Papillary Carcinoma | 1 | (2.8) | 0 | (0.0) | 0 | (0.0) | 0 | (0.0) | 1 | (0.8) |
| Of Endomertium | | | | | | | | | | |
| Sigmoid Adenocarcinoma | 0 | (0.0) | 1 | (1.3) | 0 | (0.0) | 0 | (0.0) | 1 | (0.8) |
| Sinus Piriformis Carcinoma | 1 | (2.8) | 0 | (0.0) | 0 | (0.0) | 1 | (5.0) | 1 | (0.8) |
| Skin Cancer, Nos | 1 | (2.8) | 0 | (0.0) | 0 | (0.0) | 0 | (0.0) | 1 | (0.8) |
| Squamous Cell Carcinoma Of | 1 | (2.8) | 0 | (0.0) | 0 | (0.0) | 0 | (0.0) | 1 | (0.8) |
| Endometrium | | | | | | | | | | |
| Synovial Sarcoma | 1 | (2.8) | 1 | (1.3) | 0 | (0.0) | 1 | (5.0) | 2 | (1.7) |
| Thoracic Malignancy | 1 | (2.8) | 0 | (0.0) | 0 | (0.0) | 0 | (0.0) | 1 | (0.8) |
| Thyroid Cancer, Nos | 0 | (0.0) | 1 | (1.3) | 0 | (0.0) | 0 | (0.0) | 1 | (0.8) |
| Triple Negative Breast | 0 | (0.0) | 3 | (3.8) | 0 | (0.0) | 0 | (0.0) | 3 | (2.5) |
| Cancer | | | | | | | | | | |
| Urachus | 1 | (2.8) | 0 | (0.0) | 0 | (0.0) | 1 | (5.0) | 1 | (0.8) |
| Uterine Cancer, Nos | 2 | (5.6) | 1 | (1.3) | 0 | (0.0) | 2 | (10.0) | 3 | (2.5) |
| Uterine Leiomyosarcoma - | 1 | (2.8) | 0 | (0.0) | 0 | (0.0) | 1 | (5.0) | 1 | (0.8) |
| High Grade | | | | | | | | | | |
| Missing | 0 | (0.0) | 0 | (0.0) | 1 | (16.7) | 0 | (0.0) | 1 | (0.8) |

In this first-in-human study, the anti-CD27 hCD27.131A antibody given as monotherapy and in combination with pembrolizumab was well tolerated and had manageable safety across all doses tested. A summary of the adverse events is listed below in Table 15. The data show that the safety profile was acceptable with manageable drug-related adverse effects.

TABLE 15
Adverse Event Summary (ASaT Population)

| | | | | | | | hCD27.131A + | | | |
|---|---|---|---|---|---|---|---|---|---|---|
| | | | | | hCD27.131A | | Pembro 200 | | | |
| | | | hCD27.131A + | | 30 mg Q3W + | | mg Q3W | | | |
| | hCD27.131A | | Pembro | | Pembro 200 | | Combination | | | |
| | Q3W | | Combination | | mg Q3W + | | Therapy | | | |
| | Monotherapy | | Therapy | | Chemotherapy | | CROSSOVER | | Total | |
| | n | (%) | n | (%) | n | (%) | n | (%) | n | (%) |
| Subjects in population | 36 | | 78 | | 6 | | 20 | | 120 | |
| with one or more adverse | 36 | (100.0) | 78 | (100.0) | 5 | (83.3) | 18 | (90.0) | 119 | (99.2) |
| events | | | | | | | | | | |
| with no adverse event | 0 | (0.0) | 0 | (0.0) | 1 | (16.7) | 2 | (10.0) | 1 | (0.8) |
| with drug-related^{†} adverse | 30 | (83.3) | 70 | (89.7) | 5 | (83.3) | 15 | (75.0) | 105 | (87.5) |
| events | | | | | | | | | | |
| with toxicity grade 3-5 | 11 | (30.6) | 21 | (26.9) | 2 | (33.3) | 5 | (25.0) | 36 | (30.0) |
| adverse events | | | | | | | | | | |
| with toxicity grade 3-5 drug- | 7 | (19.4) | 10 | (12.8) | 2 | (33.3) | 2 | (10.0) | 21 | (17.5) |
| related adverse events | | | | | | | | | | |
| with serious adverse events | 4 | (11.1) | 17 | (21.8) | 1 | (16.7) | 5 | (25.0) | 27 | (22.5) |
| with serious drug-related | 0 | (0.0) | 3 | (3.8) | 0 | (0.0) | 2 | (10.0) | 5 | (4.2) |
| adverse events | | | | | | | | | | |
| who died | 0 | (0.0) | 1 | (1.3) | 0 | (0.0) | 1 | (5.0) | 2 | (1.7) |
| who died due to a drug- | 0 | (0.0) | 0 | (0.0) | 0 | (0.0) | 1 | (5.0) | 1 | (0.8) |
| related adverse event | | | | | | | | | | |
| discontinued drug due to an | 2 | (5.6) | 8 | (10.3) | 1 | (16.7) | 2 | (10.0) | 13 | (10.8) |
| adverse event | | | | | | | | | | |
| discontinued drug due to a | 1 | (2.8) | 6 | (7.7) | 0 | (0.0) | 2 | (10.0) | 9 | (7.5) |
| drug-related adverse event | | | | | | | | | | |
| discontinued drug due to a | 1 | (2.8) | 2 | (2.6) | 0 | (0.0) | 1 | (5.0) | 4 | (3.3) |
| serious adverse event | | | | | | | | | | |
| discontinued drug due to a | 0 | (0.0) | 1 | (1.3) | 0 | (0.0) | 1 | (5.0) | 2 | (1.7) |
| serious drug-related adverse | | | | | | | | | | |
| event | | | | | | | | | | |

^{†}Determined by the investigator to be related to the drug.
Non-serious adverse events up to 30 days of last dose and serious adverse events up to 90 days of last dose are included.
MedDRA preferred terms “Neoplasm Progression” and “Malignant Neoplasm Progression” not related to the drug are excluded.

TABLE 16
Summary of Dose Limiting Toxicity - DLT Evaluable Population (Initial Treatment Phase in Dose Escalation)

| | | | Subject with DLT | | | | | | | |
|---|---|---|---|---|---|---|---|---|---|---|
| | | | | | PAVA^{§} | | | | | |
| | | | | | Estimate | DLT by Maximum Grade^{†} | | | | |
| Treatment Group | N | Dose Limiting Toxicity | n | % (80% CI)^{‡} | (%) | Grade 1 | Grade 2 | Grade 3 | Grade 4 | Grade 5 |
| hCD27.131A Q3W (Dose | 25 | TOTAL | 3 | 12.0 (5.4, 21.8) | 6.7 | | | | | |
| Escalation) | | | | | | | | | | |
| | | Infusion related reaction | 3 | 12.0 | | 0 | 2 | 1 | 0 | 0 |
| hCD27.131A Q3W + Pembro | 30 | TOTAL | 1 | 3.3 (0.6, 9.7) | 6.7 | | | | | |
| 200 mg Q3W (Dose | | | | | | | | | | |
| Escalation) | | | | | | | | | | |
| | | Infusion related reaction | 1 | 3.3 | | 0 | 1 | 0 | 0 | 0 |
| hCD27.131A 30 mg Q3W + | 5 | TOTAL | 0 | 0.0 (0.0, 23.5) | 6.7 | | | | | |
| Pembro 200 mg Q3W + | | | | | | | | | | |
| Chemotherapy (Dose | | | | | | | | | | |
| Escalation-Lung) | | | | | | | | | | |

DLT = Dose Limiting Toxicity
Adverse event terms are based on MedDRA version 23.0
Grades are based on NCI CTCAE version 4.03
Subjects are considered not DLT evaluable: If the subjects are allocated but not treated; or the subjects discontinue from the study prior to completing all the safety evaluations in Cycle 1 for reasons other than treatment-related AEs; and/or the subjects receive less than 75% of the total hCD27.131A and/or pembrolizumab infusions in Cycle 1 (eg, because the infusion had to be discontinued due to an infusion reaction) and do not experience a DLT.
^{†}Only the highest reported grade for a given DLT is counted for the individual subject.
^{‡}80% confidence interval is based on the Bayesian posterior confidence interval.
^{§}PAVA: Pooled-adjacent-violator algorithm.

TABLE 17
Summary of Time to Response and Duration of Response
Based on RECIST 1.1 per Investigator Assessment
in Subjects with Confirmed Response FAS Population
(Initial Treatment Phase in Dose Escalation)

| | hCD27.131A | hCD27.131A Q3W + | |
|---|---|---|---|
| | Q3W (Dose | Pembro 200 mg Q3W | |
| | Escalation) | (Dose Escalation) | Total |
| | (N = 25) | (N = 30) | (N = 55) |
| Number of | 1 | 3 | 4 |
| subjects | | | |
| with response^{†} | | | |

Time to Response (months)

| | | | | | | |
|---|---|---|---|---|---|---|
| Mean (SD) | 2.0 | (.) | 3.9 | (2.1) | 3.4 | (2.0) |
| Median | 2.0 | (2.0-2.0) | 4.0 | (1.8-6.0) | 3.0 | (1.8-6.0) |
| (Range) | | | | | | |

Response Duration^{‡} (months)

| | | | | | | |
|---|---|---|---|---|---|---|
| Median | 4.1 | (4.1-4.1) | 7.7 | (5.1-8.3+) | 6.4 | (4.1-8.3+) |
| (Range) | | | | | | |

Number (%^{‡}) of Subjects with Extended Response Duration:

| | | | | | | |
|---|---|---|---|---|---|---|
| ≥3 months | 1 | (100.0) | 3 | (100.0) | 4 | (100.0) |
| ≥6 months | 0 | (NR) | 2 | (66.7) | 2 | (50.0) |

^{†}Includes subjects with confirmed complete response or partial response.
^{‡}From product-limit (Kaplan-Meier) method for censored data.
+indicates there is no progressive disease by the time of last disease assessment.
NR = Not Reached.

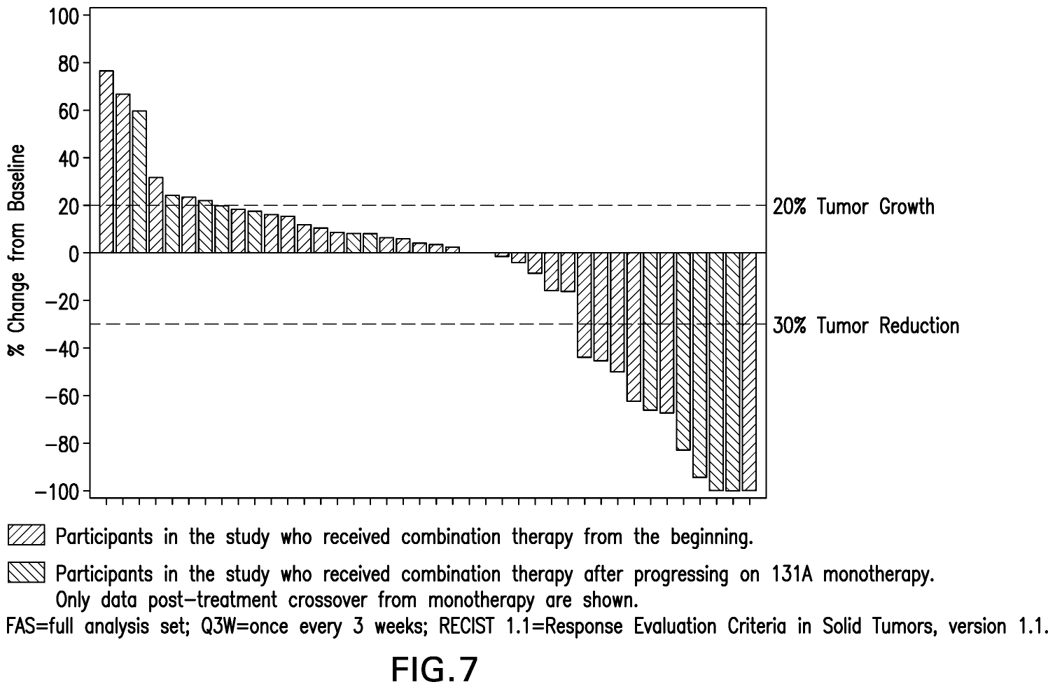

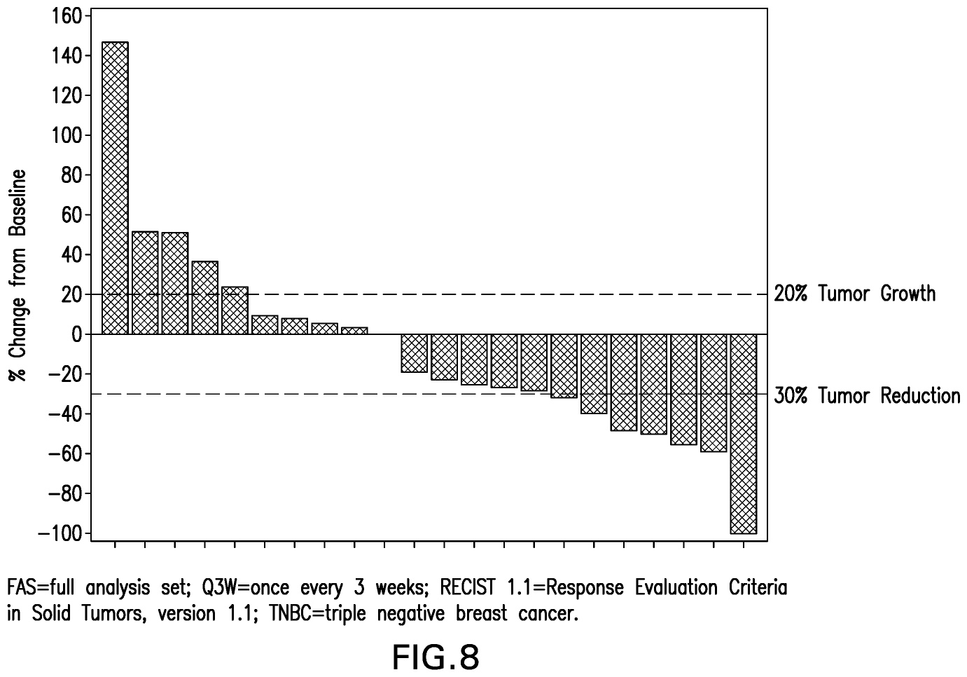

Dose escalation data were analyzed for each prespecified dose level for the subjects administered the anti-anti-CD27 hCD27.131A antibody and pembrolizumab combination treatment. DLT analysis was also performed. See Table 16. The DLT summary in Table 16 shows data for initial treatment phase in the dose escalation part of the study. There was no DLT observed in the crossover phase in the dose escalation part of the study. The disposition of the patients on monotherapy and combination therapy was also analyzed. The duration of response is shown in Table 17. Exemplary waterfall plots of percent target lesion change from baseline in participants who received escalating doses of hCD27.131A antibody as combination therapy with pembrolizumab are shown in FIG. 7. Preliminary efficacy data were available for patients with TNBC treated with 30 mg antibody hCD27.131A Q3W in combination with 200 mg pembrolizumab Q3W as part of the dose expansion portion study. An exemplary waterfall plot of maximum target lesion change from baseline based on investigator assessment per RECIST 1.1 (for TNBC: 30 mg of antibody hCD27.131A antibody Q3W+200 mg of Pembrolizumab Q3W) is shown in FIG. 8. Preliminary efficacy data was also obtained for those patients having endometrial cancer, and these data are included in Table 18.

In summary, investigators observed anti-tumor activity in subject treated with hCD27.131A antibody as a monotherapy and in a combination therapy with pembrolizumab across multiple treatment arms. Responses were observed in patients who crossed over from monotherapy to combination therapy. A summary of the anti-tumor activity (RECIST v1.1, Investigator) is shown in Table 18, Table 19, and Table 20.

TABLE 18
Summary of Best Overall Response Based on Investigator Assessment
FAS Population (Initial Treatment Phase in Dose Expansion)

| | hCD27.131A 30 mg | | | | | | hCD27.131A 30 mg | | | hCD27.131A 30 mg | | |
|---|---|---|---|---|---|---|---|---|---|---|---|---|
| | Q3W + Pembro 200 mg | | | hCD27.131A 30 mg | | | Q3W + Pembro 200 mg | | | Q6W + Pembro 400 mg | | |
| | Q3W (Dose Expansion- | | | Q3W (Dose Expansion- | | | Q3W (Dose Expansion- | | | Q6W (Dose Expansion- | | |
| | TNBC) | | | Endometrial) | | | Endometrial) | | | Endometrial) | | |
| Response Evaluation | n | % | 95% CI^{†} | n | % | 95% CI^{†} | n | % | 95% CI^{†} | n | % | 95% CI^{†} |

RECISIT 1.1 with response confirmation

| | | | | | | | | | | | | |
|---|---|---|---|---|---|---|---|---|---|---|---|---|
| Subjects in population | 27 | | | 11 | | | 11 | | | 10 | | |
| Complete Response | 0 | 0.0 | (0.0, 12.8) | 0 | 0.0 | (0.0, 28.5) | 0 | 0.0 | (0.0, 28.5) | 0 | 0.0 | (0.0, 30.8) |
| (CR) | | | | | | | | | | | | |
| Partial Response (PR) | 4 | 14.8 | (4.2, 33.7) | 0 | 0.0 | (0.0, 28.5) | 0 | 0.0 | (0.0, 28.5) | 0 | 0.0 | (0.0, 30.8) |
| Objective Response | 4 | 14.8 | (4.2, 33.7) | 0 | 0.0 | (0.0, 28.5) | 0 | 0.0 | (0.0, 28.5) | 0 | 0.0 | (0.0, 30.8) |
| (CR + PR) | | | | | | | | | | | | |
| Stable Disease (SD) | 6 | 22.2 | (8.6, 42.3) | 1 | 9.1 | (0.2, 41.3) | 3 | 27.3 | (6.0, 61.0) | 3 | 30.0 | (6.7, 65.2) |
| Disease Control (SD ≥ 6 | 5 | 18.5 | (6.3, 38.1) | 0 | 0.0 | (0.0, 28.5) | 0 | 0.0 | (0.0, 28.5) | 1 | 10.0 | (0.3, 44.5) |
| months + CR + PR) | | | | | | | | | | | | |
| Progressive Disease | 16 | 59.3 | (38.8, 77.6) | 8 | 72.7 | (39.0, 94.0) | 5 | 45.5 | (16.7, 76.6) | 4 | 40.0 | (12.2, 73.8) |
| (PD) | | | | | | | | | | | | |
| Non-evaluable (NE)^{‡} | 0 | 0.0 | (0.0, 12.8) | 0 | 0.0 | (0.0, 28.5) | 0 | 0.0 | (0.0, 28.5) | 0 | 0.0 | (0.0, 30.8) |
| No Assessment ^{††} | 1 | 3.7 | (0.1, 19.0) | 2 | 18.2 | (2.3, 51.8) | 3 | 27.3 | (6.0, 61.0) | 3 | 30.0 | (6.7, 65.2) |

RECISIT 1.1 without response confirmation

| | | | | | | | | | | | | |
|---|---|---|---|---|---|---|---|---|---|---|---|---|
| Subjects in population | 27 | | | 11 | | | 11 | | | 10 | | |
| Complete Response | 0 | 0.0 | (0.0, 12.8) | 0 | 0.0 | (0.0, 28.5) | 0 | 0.0 | (0.0, 28.5) | 0 | 0.0 | (0.0, 30.8) |
| (CR) | | | | | | | | | | | | |
| Partial Response (PR) | 5 | 18.5 | (6.3, 38.1) | 0 | 0.0 | (0.0, 28.5) | 0 | 0.0 | (0.0, 28.5) | 0 | 0.0 | (0.0, 30.8) |
| Objective Response | 5 | 18.5 | (6.3, 38.1) | 0 | 0.0 | (0.0, 28.5) | 0 | 0.0 | (0.0, 28.5) | 0 | 0.0 | (0.0, 30.8) |
| (CR + PR) | | | | | | | | | | | | |
| Stable Disease (SD) | 5 | 18.5 | (6.3, 38.1) | 1 | 9.1 | (0.2, 41.3) | 3 | 27.3 | (6.0, 61.0) | 3 | 30.0 | (6.7, 65.2) |
| Disease Control (SD ≥ 6 | 5 | 18.5 | (6.3, 38.1) | 0 | 0.0 | (0.0, 28.5) | 0 | 0.0 | (0.0, 28.5) | 1 | 10.0 | (0.3, 44.5) |
| months + CR + PR) | | | | | | | | | | | | |
| Progressive Disease | 16 | 59.3 | (38.8, 77.6) | 8 | 72.7 | (39.0, 94.0) | 5 | 45.5 | (16.7, 76.6) | 4 | 40.0 | (12.2, 73.8) |
| (PD) | | | | | | | | | | | | |
| Non-evaluable (NE)^{‡} | 0 | 0.0 | (0.0, 12.8) | 0 | 0.0 | (0.0, 28.5) | 0 | 0.0 | (0.0, 28.5) | 0 | 0.0 | (0.0, 30.8) |
| No Assessment ^{††} | 1 | 3.7 | (0.1, 19.0) | 2 | 18.2 | (2.3, 51.8) | 3 | 27.3 | (6.0, 61.0) | 3 | 30.0 | (6.7, 65.2) |

^{†}Based on Clopper-Pearson confidence interval method.
^{‡}Not-evaluable (NE) includes subjects when confirmation of CR and PR is required, includes the following subjects based on RECIST 1.1guideline: (1) subjects with no imaging/measurement done at all or only partial lesion measurements are done at both the first time point and subsequent time point; (2) subjects who are CR at first time point but with no imaging/measurement done at all or only partial lesion measurements are done without meeting the minimum criteria for SD duration at subsequent time point; (3) subjects who are PR at first time point but with no imaging/measurement done at all or only partial lesion measurements are done without meeting the minimum criteria for SD duration at subsequent time point.
^{††} No Assessment includes subjects without post-baseline assessment on the data cutoff date.

TABLE 19
Summary of Best Overall Response Based on Investigator Assessment
FAS Population (Initial Treatment Phase in Dose Escalation)

| | | | | hCD27.131A Q3W + Pembro 200 mg | | |
|---|---|---|---|---|---|---|
| | hCD27.131A Q3W (Dose Escalation) | | | Q3W (Dose Escalation)) | | |
| Response Evaluation | n | % | 95% CI^{†} | n | % | 95% CI^{†} |

RECISIT 1.1 with response confirmation

| | | | | | | |
|---|---|---|---|---|---|---|
| Subjects in population | 25 | | | 30 | | |
| Complete Response (CR) | 0 | 0.0 | (0.0, 13.7) | 0 | 0.0 | (0.0, 11.6) |
| Partial Response (PR) | 1 | 4.0 | (0.1, 20.4) | 3 | 10.0 | (2.1, 26.5) |
| Objective Response (CR + PR) | 1 | 4.0 | (0.1, 20.4) | 3 | 10.0 | (2.1, 26.5) |
| Stable Disease (SD) | 5 | 20.0 | (6.8, 40.7) | 12 | 40.0 | (22.7, 59.4) |
| Disease Control (SD ≥ 6 | 3 | 12.0 | (2.5, 31.2) | 9 | 30.0 | (14.7, 49.4) |
| months + CR + PR) | | | | | | |
| Progressive Disease (PD) | 16 | 64.0 | (42.5, 82.0) | 12 | 40.0 | (22.7, 59.4) |
| Non-evaluable (NE)^{‡} | 0 | 0.0 | (0.0, 13.7) | 0 | 0.0 | (0.0, 11.6) |
| No Assessment ^{††} | 3 | 12.0 | (2.5, 31.2) | 3 | 10.0 | (2.1, 26.5) |

RECISIT 1.1 without response confirmation

| | | | | | | |
|---|---|---|---|---|---|---|
| Subjects in population | 25 | | | 30 | | |
| Complete Response (CR) | 0 | 0.0 | (0.0, 13.7) | 1 | 3.3 | (0.1, 17.2) |
| Partial Response (PR) | 1 | 4.0 | (0.1, 20.4) | 4 | 13.3 | (3.8, 30.7) |
| Objective Response (CR + PR) | 1 | 4.0 | (0.1, 20.4) | 5 | 16.7 | (5.6, 34.7) |
| Stable Disease (SD) | 5 | 20.0 | (6.8, 40.7) | 10 | 33.3 | (17.3, 52.8) |
| Disease Control (SD ≥ 6 | 3 | 12.0 | (2.5, 31.2) | 9 | 30.0 | (14.7, 49.4) |
| months + CR + PR) | | | | | | |
| Progressive Disease (PD) | 16 | 64.0 | (42.5, 82.0) | 12 | 40.0 | (22.7, 59.4) |
| Non-evaluable (NE)^{‡} | 0 | 0.0 | (0.0, 13.7) | 0 | 0.0 | (0.0, 11.6) |
| No Assessment ^{††} | 3 | 12.0 | (2.5, 31.2) | 3 | 10.0 | (2.1, 26.5) |

^{†}Based on Clopper-Pearson confidence interval method.
^{‡}Not-evaluable (NE) includes subjects when confirmation of CR and PR is required, includes the following subjects based on RECIST 1.1guideline: (1) subjects with no imaging/measurement done at all or only partial lesion measurements are done at both the first time point and subsequent time point; (2) subjects who are CR at first time point but with no imaging/measurement done at all or only partial lesion measurements are done without meeting the minimum criteria for SD duration at subsequent time point; (3) subjects who are PR at first time point but with no imaging/measurement done at all or only partial lesion measurements are done without meeting the minimum criteria for SD duration at subsequent time point.
^{‡‡} No Assessment includes subjects without post-baseline assessment on the data cutoff date.

TABLE 20
Summary of Best Overall Response Based on Investigator Assessment
FAS Population (Crossover Phase in Dose Escalation)

| | hCD27.131A Q3W + Pembro 200 mg | | |
|---|---|---|---|
| | Q3W (Dose Escalation) | | |
| Response Evaluation | n | % | 95% CI^{†} |

RECISIT 1.1 with response confirmation

| | | | |
|---|---|---|---|
| Subjects in population | 13 | | |
| Complete Response (CR) | 2 | 15.4 | (1.9, 45.4) |
| Partial Response (PR) | 2 | 15.4 | (1.9, 45.4) |
| Objective Response (CR + PR) | 4 | 30.8 | (9.1, 61.4) |
| Stable Disease (SD) | 2 | 15.4 | (1.9, 45.4) |
| Disease Control (SD ≥ 6 | 4 | 30.8 | (9.1, 61.4) |
| months + CR + PR) | | | |
| Progressive Disease (PD) | 7 | 53.8 | (25.1, 80.8) |
| Non-evaluable (NE)^{‡} | 0 | 0.0 | (0.0, 24.7) |
| No Assessment ^{††} | 0 | 0.0 | (0.0, 24.7) |

RECISIT 1.1 without response confirmation

| | | | |
|---|---|---|---|
| Subjects in population | 13 | | |
| Complete Response (CR) | 2 | 15.4 | (1.9, 45.4) |
| Partial Response (PR) | 3 | 23.1 | (5.0, 53.8) |
| Objective Response (CR + PR) | 5 | 38.5 | (13.9, 68.4) |
| Stable Disease (SD) | 1 | 7.7 | (0.2, 36.0) |
| Disease Control (SD ≥ 6 | 5 | 38.5 | (13.9, 68.4) |
| months + CR + PR) | | | |
| Progressive Disease (PD) | 7 | 53.8 | (25.1, 80.8) |
| Non-evaluable (NE)^{‡} | 0 | 0.0 | (0.0, 24.7) |
| No Assessment ^{††} | 0 | 0.0 | (0.0, 24.7) |

^{†}Based on Clopper-Pearson confidence interval method.
^{‡}Not-evaluable (NE) includes subjects when confirmation of CR and PR is required, includes the following subjects based on RECIST 1.1guideline: (1) subjects with no imaging/measurement done at all or only partial lesion measurements are done at both the first time point and subsequent time point; (2) subjects who are CR at first time point but with no imaging/measurement done at all or only partial lesion measurements are done without meeting the minimum criteria for SD duration at subsequent time point; (3) subjects who are PR at first time point but with no imaging/measurement done at all or only partial lesion measurements are done without meeting the minimum criteria for SD duration at subsequent time point.
^{††} No Assessment includes subjects without post-baseline assessment on the data cutoff date.

1. A method for treating cancer in a patient in need thereof comprising administering to the patient about 2 mg to about 700 mg of an anti-CD27 antibody or antigen binding fragment thereof comprising a heavy chain and a light chain, wherein the light chain comprises light chain CDRs comprising the amino acid sequences of SEQ ID NOs: 4, 5 and 6, respectively and the heavy chain comprises heavy chain CDRs comprising the amino acid sequences of SEQ ID NOs: 1, 2 and 3, respectively.

2. The method of claim 1, wherein the anti-CD27 antibody or antigen binding fragment thereof is administered via intravenous infusion.

3. The method of claim 1, wherein the patient is administered about 2 mg, about 7 mg, about 20 mg, about 30 mg, about 70 mg, about 200 mg, or about 200 mg to about 700 mg of the anti-CD27 antibody or antigen binding fragment thereof.

4. (canceled)

5. The method of claim 1, wherein the patient is administered about 30 mg of the anti-CD27 antibody or antigen binding fragment thereof.

6. The method of claim 1, wherein the patient is administered 30 mg of the anti-CD27 antibody or antigen binding fragment thereof.

7. (canceled)

8. (canceled)

9. (canceled)

10. The method of claim 1, wherein the patient is administered a dose of 2 mg, 7 mg, 20 mg, 30 mg, 200 mg, or 200 mg to 700 mg of the anti-CD27 antibody or antigen binding fragment thereof.

11. The method of claim 1, wherein the patient is administered the anti-CD27 antibody or antigen binding fragment thereof on Day 1 and then at least once about 3 weeks to about 6 weeks thereafter.

12. The method of claim 1, wherein the anti-CD27 antibody or antigen binding fragment thereof comprises a heavy chain and a light chain, and wherein the heavy chain comprises a heavy chain variable region comprising SEQ ID NO.:7 and the light chain comprises a light chain variable region comprising SEQ ID NO.: 9.

13. The method of claim 1, wherein the anti-CD27 antibody or antigen binding fragment thereof comprises a heavy chain and a light chain, and wherein the heavy chain comprises the amino acid sequence of SEQ ID NO.:8 and the light chain comprises the amino acid sequence of SEQ ID NO.:10.

14. (canceled)

15. A method for treating cancer in a patient in need thereof, wherein an anti-CD27 antibody or antigen binding fragment thereof is co-administered with an anti-PD-1 antibody or anti-PD-L1 antibody or antigen binding fragment thereof, wherein the anti-CD27 antibody or antigen binding fragment thereof comprises a heavy chain and a light chain wherein the light chain comprises light chain CDRs comprising the amino acid sequences of SEQ ID NOs: 4, 5 and 6, respectively and the heavy chain comprises heavy chain CDRs comprising the amino acid sequences of SEQ ID NOs: 1, 2 and 3, respectively.

16. The method of claim 15, wherein the anti-CD27 antibody or antigen binding fragment thereof is co-formulated with an anti-PD-1 antibody or anti-PD-L1 antibody or antigen binding fragment thereof.

17. The method of claim 15, wherein the anti-PD-1 antibody, or antigen binding fragment thereof, specifically binds to human PD-1 and blocks the binding of human PD-L1 to human PD-1.

18. The method of claim 17, wherein the anti-PD-1 antibody, or antigen binding fragment thereof, also blocks binding of human PD-L2 to human PD-1.

19. The method of claim 18, wherein the anti-PD-1 antibody, or antigen binding fragment thereof comprises: (a) light chain CDRs of SEQ ID NOs: 11, 12 and 13, and (b) heavy chain CDRs of SEQ ID NOs: 16, 17 and

20. The method of claim 19, wherein the anti-PD-1 antibody comprises a heavy chain and a light chain, and wherein the heavy chain comprises a heavy chain variable region comprising the amino acid sequence of SEQ ID NO.:19 and the light chain comprises a light chain variable region comprising the amino acid sequence of SEQ ID NO.: 14.

21. (canceled)

22. (canceled)

23. The method of claim 15, wherein the anti-PD-1 antibody is pembrolizumab.

24. The method of claim 15, wherein the anti-PD-1 antibody is nivolumab.

25. The method of claim 15, wherein the anti-PD-L1 antibody is atezolizumab, durvalumab, or avelumab.

26. The method of claim 1, wherein the anti-CD27 antibody or antigen binding fragment thereof is administered every 3 weeks (Q3W).

27. The method of claim 1, wherein the anti-CD27 antibody or antigen binding fragment thereof is administered every 6 weeks (Q6W).

28. The method of claim 26, wherein the anti-PD-1 antibody is administered at 200 mg via intravenous infusion on Day 1 and then once every three weeks thereafter.

29. The method of claim 27, wherein the anti-PD-1 antibody is administered at 400 mg via intravenous infusion on Day 1 and then once every six weeks thereafter.

30. (canceled)

31. The method of claim 15, wherein the anti-PD-1 antibody is a humanized anti-PD-1 antibody that comprises a heavy chain and a light chain, and wherein the heavy chain comprises a heavy chain variable region comprising heavy chain CDRs of SEQ ID NOs: 16, 17 and 18, respectively, and the light chain comprises a light chain variable region comprising light chain CDRs comprising the amino acid sequences of SEQ ID NOs: 11, 12 and 13, respectively; and the anti-CD27 antibody is a humanized anti-CD27 antibody which comprises a heavy chain and a light chain, and wherein the heavy chain comprises a heavy chain variable region comprising heavy chain CDRs comprising the amino acid sequences of SEQ ID NOs: 1, 2 and 3, respectively, and the light chain comprises a light chain variable region comprising light chain CDRs comprising the amino acid sequences of SEQ ID NOs: 4, 5 and 6, respectively.

32. The method of claim 15, wherein the anti-PD-1 antibody comprises a heavy chain and a light chain, and wherein the heavy chain comprises a heavy chain variable region comprising the amino acid sequence of SEQ ID NO.: 19 and the light chain comprises a light chain variable region comprising the amino acid sequence of SEQ ID NO.: 14; and the anti-CD27 antibody comprises a heavy chain and a light chain, and wherein the heavy chain comprises a heavy chain variable region comprising the amino acid sequence of SEQ ID NO.:7 and the light chain comprises a light chain variable region comprising the amino acid sequence of SEQ ID NO.: 9.

33. The method of claim 15, wherein the anti-PD-1 antibody comprises a heavy chain and a light chain, and wherein the heavy chain comprises the amino acid sequence of SEQ ID NO.:20 and the light chain comprises the amino acid sequence of SEQ ID NO.: 15; and the anti-CD27 antibody comprises a heavy chain and a light chain, and wherein the heavy chain comprises the amino acid sequence of SEQ ID NO.: 8 and the light chain comprises the amino acid sequence of SEQ ID NO.: 10.

34. The method of claim 15, wherein the anti-PD-1 antibody is administered at 200 mg via intravenous infusion on Day 1 and then once every three weeks thereafter, and the anti-CD27 antibody is administered at 30 mg via intravenous infusion on Day 1 and then once every three weeks thereafter.

35. The method of claim 15, wherein the anti-PD-1 antibody is administered at 400 mg via intravenous infusion on Day 1 and then once every six weeks thereafter, and the anti-CD27 antibody is administered at 30 mg via intravenous infusion on Day 1 once every six weeks.

36. (canceled)

37. The method of claim 1, wherein the cancer comprises a solid tumor.

38. The method of claim 1, wherein the cancer is selected from the group consisting of: triple-negative breast cancer (TNBC), non-squamous non-small cell lung cancer (NSCLC), and endometrial cancer.

39. The method of claim 1, wherein the method further comprises administering a carboplatin and/or pemetrexed.

40. (canceled)

41. The method of claim 1, wherein the patient is administered an analgesic and/or an antihistamine prior to the anti-CD27 antibody or antigen binding fragment thereof.

42. (canceled)

43. (canceled)

44. (canceled)

45. (canceled)

46. (canceled)


SEQUENCE LISTING (20 sequences)

SEQ ID NO: 1 - protein, 5 aa, Artificial Sequence
  hCD27.131A H - CDR1
NYGMN

SEQ ID NO: 2 - protein, 17 aa, Artificial Sequence
  hCD27.131A H - CDR2
WINTNTGEPTYAEEFKG

SEQ ID NO: 3 - protein, 7 aa, Artificial Sequence
  hCD27.131A H - CDR3
EGDAMDY

SEQ ID NO: 4 - protein, 10 aa, Artificial Sequence
  hCD27.131A L - CDR1
SASSSVSYMH

SEQ ID NO: 5 - protein, 7 aa, Artificial Sequence
  hCD27.131A L - CDR2
DTSKLAS

SEQ ID NO: 6 - protein, 9 aa, Artificial Sequence
  hCD27.131A L - CDR3
QQWNSYPFT

SEQ ID NO: 7 - protein, 116 aa, Artificial Sequence [sequence omitted]

SEQ ID NO: 8 - protein, 116 aa, Artificial Sequence [sequence omitted]

SEQ ID NO: 9 - protein, 106 aa, Artificial Sequence
  hCD27.131A variable light region/domain
EIVLTQSPATLSLSPGERATLSCSASSSVSYMHWYQQKPGQAPKRWIYDTSKLASGVPARFSGSGSGTDYSLTISSLEPEDFAVYYCQQWNSYPFTFGQGTKLEIK

SEQ ID NO: 10 - protein, 213 aa, Artificial Sequence
  hCD27.131A light chain
EIVLTQSPATLSLSPGERATLSCSASSSVSYMHWYQQKPGQAPKRWIYDTSKLASGVPARFSGSGSGTDYSLTISSLEPEDFAVYYCQQWNSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 15 aa, Artificial Sequence
  CDR1
RASKGVSTSGYSYLH

SEQ ID NO: 12 - protein, 7 aa, Artificial Sequence
  CDR2
LASYLES

SEQ ID NO: 13 - protein, 9 aa, Artificial Sequence
  CDR3
QHSRDLPLT

SEQ ID NO: 14 - protein, 111 aa, Artificial Sequence [sequence omitted]

SEQ ID NO: 15 - protein, 218 aa, Artificial Sequence
  Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, Artificial Sequence
  CDR1
NYYMY

SEQ ID NO: 17 - protein, 17 aa, Artificial Sequence
  CDR2
GINPSNGGTNFNEKFKN

SEQ ID NO: 18 - protein, 11 aa, Artificial Sequence
  CDR3
RDYRFDMGFDY

SEQ ID NO: 19 - protein, 120 aa, Artificial Sequence [sequence omitted]

SEQ ID NO: 20 - protein, 350 aa, Artificial Sequence
  Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYT